(19) United States
(12) Patent Application Publication
(10) Pub. No.: US 2024/0294984 A1
(43) Pub. Date: Sep. 5, 2024

(54) METHODS AND SYSTEMS FOR ALLELE TYPING

(71) Applicant: GUARDANT HEALTH, INC., Palo Alto, CA (US)

(72) Inventor: Sante GNERRE, Mountain View, CA (US)

(21) Appl. No.: 18/618,588

(22) Filed: Mar. 27, 2024

Related U.S. Application Data
(63) Continuation of application No. PCT/US2023/065469, filed on Apr. 6, 2023.
(60) Provisional application No. 63/494,724, filed on Apr. 6, 2023.
(60) Provisional application No. 63/487,926, filed on Mar. 2, 2023.

(51) Int. Cl. C12Q 1/6876; C12Q 1/6869

(52) U.S. Cl. CPC C12Q 1/6876; C12Q 1/6869; C12Q 2600/156

(57) ABSTRACT

Provided herein are methods and systems for allele typing and variant calling.

### CROSS REFERENCE TO RELATED APPLICATION

This application is a Continuation of PCT Application No. PCT/US2023/065469, filed Apr. 6, 2023, which claims the benefit of U.S. Provisional Application No. 63/228,681, filed Apr. 7, 2022, U.S. Provisional Application No. 63/487,926, filed Mar. 2, 2023, and also claims the benefit of U.S. Provisional Application No. 63/494,724, filed Apr. 6, 2023. Each are incorporated by reference in their entirety for all purposes.

### BACKGROUND

Human leukocyte antigen (HLA) genes, which encode major histocompatibility complex proteins in humans, are located in the short arm of chromosome 6. These encoded HLA proteins are displayed on the cell surface and can be classified into two distinct classes. Class I HLA proteins (A, B, and C) present intracellular antigens originating from viruses or tumors to cytotoxic T lymphocytes. Class II HLA proteins (DR, DQ, and DP) present extracellular antigens to T-helper cells. HLA genes are highly polymorphic and play an important role in immune-mediated diseases, tumor-development processes, transplanted organ or tissue survival determination, and drug hypersensitivity.

HLA genotyping is a complex procedure due to the extreme degree of polymorphism in the major histocompatibility complex family. The most polymorphic regions, known as the core exons, are exons 2 and 3 in HLA class I genes and exon 2 in HLA class II genes. The sequences of the core exons are the most popular targets for genotyping as they are believed to be essential determinants of antigen specificity, which is informative for transplantation. However, in population genetic and evolutionary studies, many polymorphisms in other exons, introns, and UTRs have been identified and contribute to creating HLA nomenclature. Currently, HLA typing is performed using DNA-based methods, including SSP- (sequence-specific primer), SSO- (sequence-specific oligonucleotide), and RFLP-PCR (restriction fragment length polymorphism polymerase chain reaction) and sequence-based typing (SBT). SBT produces uncertain results due to insufficient sequencing and ambiguous haplotype phasing.

Accordingly, there is a need for methods of for accurate allele typing using sequence information.

### SUMMARY

Disclosed are methods of allele typing. Disclosed are methods of treatment based on allele typing.

Disclosed are methods comprising determining a plurality of known allele sequences, determining a plurality of sequence reads of a target region of a chromosome of a subject, wherein the target region of the chromosome comprises one or more loci, aligning the plurality of sequence reads to the plurality of known allele sequences, determining, based on the alignment, for each known allele sequence of the plurality of known allele sequences, a number of sequence reads that aligned to each known allele sequence, and determining, based on the numbers of sequence reads that aligned to each known allele sequence, for the one or more loci, the known allele sequences present at the one or more loci.

Disclosed are methods comprising determining a plurality of known allele sequences, determining a plurality of sequence reads of a target region of a chromosome of a subject, wherein the target region of the chromosome comprises one or more loci, aligning the plurality of sequence reads to the plurality of known allele sequences, determining, based on the alignment, for each known allele sequence of the plurality of known allele sequences, a number of sequence read families (i.e., number of nucleic acid molecules—a sequence read family may be a group of sequence reads corresponding to a single nucleic acid molecule) that aligned to each known allele sequence, and determining, based on the numbers of sequence read families that aligned to each known allele sequence, for the one or more loci, the known allele sequences present at the one or more loci.

Disclosed are methods comprising determining a plurality of known human leukocyte antigen (HLA) allele sequences, determining a plurality of sequence reads of a target region of a chromosome of a subject, wherein the target region of the chromosome comprises one or more loci, aligning the plurality of sequence reads to the plurality of known HLA allele sequences, determining, based on the alignment, for each known HLA allele sequence of the plurality of known HLA allele sequences, a number of sequence reads that aligned to each known HLA allele sequence, generating, based on the numbers of sequence reads that aligned to each known HLA allele sequence, one or more supersets of known HLA allele sequences, and determining, based on a number of distinct reads in the one or more supersets of known HLA allele sequences, for the one or more loci, the known HLA allele sequences present at the one or more loci.

Disclosed are methods comprising determining a plurality of known human leukocyte antigen (HLA) allele sequences, determining a plurality of sequence reads of a target region of a chromosome of a subject, wherein the target region of the chromosome comprises one or more loci, aligning the plurality of sequence reads to the plurality of known HLA allele sequences, determining, based on the alignment, for each known HLA allele sequence of the plurality of known HLA allele sequences, a number of sequence read families that aligned to each known HLA allele sequence, generating, based on the numbers of sequence read families that aligned to each known HLA allele sequence, one or more supersets of known HLA allele sequences, and determining, based on a number of distinct read families in the one or more supersets of known HLA allele sequences, for the one or more loci, the known HLA allele sequences present at the one or more loci.

Disclosed are methods comprising determining a plurality of known killer cell immunoglobulin-like receptor (KIR) allele sequences, determining a plurality of sequence reads of a target region of a chromosome of a subject, wherein the target region of the chromosome comprises one or more loci, aligning the plurality of sequence reads to the plurality of known KIR allele sequences, determining, based on the alignment, for each known KIR allele sequence of the plurality of known KIR allele sequences, a number of sequence reads that aligned to each known KIR allele sequence, generating, based on the numbers of sequence reads that aligned to each known KIR allele sequence, one or more supersets of known KIR allele sequences, and determining, based on a number of distinct reads in the one or more supersets of known KIR allele sequences, for the one or more loci, the known KIR allele sequences present at the one or more loci.

Disclosed are methods comprising determining a plurality of known killer cell immunoglobulin-like receptor (KIR) allele sequences, determining a plurality of sequence reads of a target region of a chromosome of a subject, wherein the target region of the chromosome comprises one or more loci, aligning the plurality of sequence reads to the plurality of known KIR allele sequences, determining, based on the alignment, for each known KIR allele sequence of the plurality of known KIR allele sequences, a number of sequence read families that aligned to each known KIR allele sequence, generating, based on the numbers of sequence read families that aligned to each known KIR allele sequence, one or more supersets of known KIR allele sequences, and determining, based on a number of distinct read families in the one or more supersets of known KIR allele sequences, for the one or more loci, the known KIR allele sequences present at the one or more loci.

Disclosed are methods comprising determining a plurality of known allele sequences, determining a plurality of sequence reads of a target region of a chromosome of a subject, wherein the target region of the chromosome comprises one or more loci, aligning the plurality of sequence reads to the plurality of known allele sequences, determining, based on the alignment, for each known allele sequence of the plurality of known allele sequences, a number of sequence reads that aligned to each known allele sequence, generating, based on the numbers of sequence reads that aligned to each known allele sequence, one or more supersets of known allele sequences, and determining, based on a number of distinct reads in the one or more supersets of known allele sequences, for the one or more loci, the known allele sequences present at the one or more loci.

Disclosed are methods comprising determining a plurality of known allele sequences, determining a plurality of sequence reads of a target region of a chromosome of a subject, wherein the target region of the chromosome comprises one or more loci, aligning the plurality of sequence reads to the plurality of known allele sequences, determining, based on the alignment, for each known allele sequence of the plurality of known allele sequences, a number of sequence read families that aligned to each known allele sequence, generating, based on the numbers of sequence read families that aligned to each known allele sequence, one or more supersets of known allele sequences, and determining, based on a number of distinct read families in the one or more supersets of known allele sequences, for the one or more loci, the known allele sequences present at the one or more loci.

In some embodiments, the results of the systems and methods disclosed herein are used as an input to generate a report. The report may be in a paper or electronic format. For example, the determination of allele type (e.g., allele sequence), as determined by the methods and systems disclosed herein, can be displayed directly in such a report.

The various steps of the methods disclosed herein, or steps executed by the systems disclosed herein, may be carried out at the same or different times, in the same or different geographical locations, e.g., countries, and/or by the same or different people.

### BRIEF DESCRIPTION OF THE FIGURES

The accompanying drawings, which are incorporated in and constitute a part of this specification, illustrate certain embodiments, and together with the written description, serve to explain certain principles of the methods, computer readable media, and systems disclosed herein. The description provided herein is better understood when read in conjunction with the accompanying drawings which are included by way of example and not by way of limitation. It will be understood that like reference numerals identify like components throughout the drawings unless the context indicates otherwise. It will also be understood that some or all of the figures may be schematic representations for purposes of illustration and do not necessarily depict the actual relative sizes or locations of the elements shown.

FIG. 1A is a flow chart that schematically depicts exemplary method steps for allele typing.

FIG. 1B is a flow chart that schematically depicts another exemplary method steps for allele typing.

FIG. 2 shows an example of a system for allele typing.

FIG. 3 shows an example k-mer data structure.

FIG. 4 shows an example Hasse diagram.

FIG. 5 shows an example graph data structure.

FIG. 6 shows an example graph data structure.

FIG. 7 shows an example graph data structure.

FIG. 8 shows an example superset comparison.

FIG. 9 shows an example method.

FIG. 10 shows an example method.

FIG. 11 shows an example of different alleles with high homology. The alleles in FIG. 11 encode distinct products at the protein level, even if most of their genomic content is identical. For example, two alleles coding for different proteins may in practice differ only on one or two bases.

FIG. 12 shows example flowcharts of the disclosed methods. The disclosed methods can be configured to run in one or more modes. For example, a “Build index” mode (to generate a kmers map database) and a “Call alleles” mode (to call alleles on an input sample).

FIG. 13 shows an example of the hierarchical structure of allele names. An HLA allele name is encoded by a string such as A*01:02:03:04, comprising a gene name (A, for HLA-A), and up to four sets of digits. When reviewing two allele names:
Matching the first two sets of digits: the alleles code for the same protein, but they may have different genomic content at the exon level.
Matching the first three sets of digits: the alleles have the same genomic content at the exon level (their bases are identical in the exons).
Matching all four sets of digits: the alleles have the same genomic content both in the exon and the introns.

FIG. 14 shows a screen capture of an Integrative Genomics Viewer showing one allele being called by Kmerizer and the set of all the supporting reads for the called allele. This view only contains sequence reads that perfectly match the called allele (regardless of alignment onto the reference genome).

FIG. 15 shows analytical performance of kmerizer on two simulated sets of 100 samples each, matched against the sensitivity of HISAT-genotype. The “Regular” set contains 100 simulated samples, with a simulated error rate of 0.1%, with reads in 14 HLA genes (HISAT-genotype does not call genes in DRB3, DRB4, and DRB5). The “Difficult” set contains 100 simulated samples, with a simulated error rate of 0.1%, with reads in 8 HLA genes, selected from a pool of alleles known to share a high level of homology.

FIG. 16 shows analytical performance of Kmerizer on a simulated set of 100 samples. This set contains 100 simulated samples, with a simulated error rate of 0.1%, with reads in 17 KIR genes.

FIG. 17 shows analytical performance of Kmerizer on 19 samples with known HLA status on 4 HLA genes (HLA-A, HLA-B, HLA-C, and HLA-DQB1), matched against the performance of HISAT-genotype.

FIG. 18 shows HLA germline allele prevalence comparison between methylation epigenomic detection samples (n=2004) and Allele Frequency Net Database (AFND) genotype data from different geographic regions.

FIG. 19 shows a distribution of counts of germline HLA alleles for which immunogenic neoantigens were predicted.

FIG. 20 shows the overall germline HLA allele distribution for all between methylation epigenomic detection samples.

FIG. 21 shows that germline allele(s) and homozygous/heterozygous status were randomly assigned based on AFND to generate germline reads using references from the IMGT/HLA database; somatic mutation was then randomly generated at an exonic position and mutant reads were then generated from the altered reference.

FIG. 22 shows variant allele frequency (AF) distribution of detected variants.

FIG. 23 shows TMB v. predicted neoepitope affinity across genes. Spearman correlations listed for each group.

FIG. 24 shows the distribution of neoantigen binding affinities by cancer type.

### I. DEFINITIONS

In order for the present disclosure to be more readily understood, certain terms are first defined below. Additional definitions for the following terms and other terms may be set forth through the specification. If a definition of a term set forth below is inconsistent with a definition in a patent application or issued patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth. It will also be appreciated that there is an implied “about” prior to the temperatures, concentrations, times, number of bases or base pairs, coverage, etc. discussed in the present disclosure, such that slight and insubstantial equivalents are within the scope of the present disclosure. In this application, the use of the singular includes the plural unless specifically stated otherwise. Also, the use of “comprise,” “comprises,” “comprising,” “contain,” “contains,” “containing,” “include,” “includes,” and “including” are not intended to be limiting.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Adapter: As used herein, “adapter” or “adaptor” refers to short nucleic acids (e.g., less than about 500, less than about 100 or less than about 50 nucleotides in length) that are typically at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequencing reads of a given nucleic acid molecule. Adapters of the same or different sequence can be linked to the respective ends of a nucleic acid molecule. In certain embodiments, the same adapter is linked to the respective ends of the nucleic acid molecule except that the nucleic acid tag differs in its sequence. In some embodiments, the adapter is a Y-shaped adapter in which one end is blunt ended or tailed as described herein, for joining to a nucleic acid molecule, which is also blunt ended or tailed with one or more complementary nucleotides. In still other exemplary embodiments, an adapter is a bell-shaped adapter that includes a blunt or tailed end for joining to a nucleic acid molecule to be analyzed. Other exemplary adapters include T-tailed and C-tailed adapters.

Administer: As used herein, “administer” or “administering” a therapeutic agent (e.g., an immunological therapeutic agent, a DNA damage response (DDR) inhibitor (e.g., a poly (ADP-ribose) polymerase (PARP) inhibitor (PARPi)), etc.) to a subject means to give, apply or bring the composition into contact with the subject. Administration can be accomplished by any of a number of routes, including, for example, topical, oral, subcutaneous, intramuscular, intraperitoneal, intravenous, intrathecal, and intradermal.

Align: As used herein, “align,” alignment,” and “aligning” in the context of nucleic acids refers to arranging sequences of DNA or RNA to identify regions of similarity. Similarity may be related to the nucleotide sequence, structural, functional, and/or evolutionary relationships between the sequences. Alignment of DNA sequences involves alignment of genomic DNA of one sequence to genomic DNA of at least one other sequence. Such alignment may exclude non-genomic DNA, such as a molecular barcode, padding bases, and the like. For example, genomic DNA of a sequence read may be aligned to genomic DNA of a reference DNA sequence, excluding any molecular tag or adapter sequence that may be attached to the sequence read.

As used herein, recitation that nucleotides “correspond to” nucleotides in a sequence refers to nucleotides identified upon alignment with the sequence to maximize identity using a standard alignment algorithm, such as the GAP algorithm.

As used herein, “sequence identity,” “sequence homology,” or “identity” refers to the number of identical or similar nucleotide bases in an alignment between two or more polynucleotide sequences. In one non-limiting example, “at least 90% identical to” refers to percent identities from 90 to 100% relative to the reference polynucleotide. Identity at a level of 90% or more is indicative of the fact that, assuming for exemplification purposes a test and reference polynucleotide length of 100 nucleotides are compared, no more than 10% (i.e., 10 out of 100) of nucleotides in the test polynucleotide differs from that of the reference polynucleotide. Such differences can be represented as point mutations randomly distributed over the entire length of a nucleotide sequence or they can be clustered in one or more locations of varying length up to the maximum allowable, e.g., 10/100 nucleotide difference (approximately 90% identity). Differences are defined as nucleic acid substitutions, insertions or deletions.

Sequence identity can be determined by sequence alignment of nucleic acid sequences to identify regions of similarity or identity. For purposes herein, sequence identity is generally determined by alignment to identify identical bases. The alignment can be local or global. Matches, mismatches and gaps can be identified between compared sequences. Gaps are null nucleotides inserted between the bases of aligned sequences so that identical or similar characters are aligned. Generally, there can be internal and terminal gaps. Sequence identity can be determined by taking into account gaps as the number of identical bases/length of the shortest sequence×100. When using gap penalties, sequence identity can be determined with no penalty for end gaps (e.g., terminal gaps are not penalized). Alternatively, sequence identity can be determined without taking into account gaps as the number of identical positions/length of the total aligned sequence×100.

As used herein, a “global alignment” is an alignment that aligns two sequences from beginning to end, aligning each base in each sequence only once. An alignment is produced regardless of whether or not there is similarity or identity between the sequences. For example, 50% sequence identity based on “global alignment” means that in an alignment of the full sequence of two compared sequences each of 100 nucleotides in length, 50% of the bases are the same. It is understood that global alignment also can be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman et al. J. Mol. Biol. 48: 443 (1970). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a “local alignment” is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Adv. Appl. Math. 2: 482 (1981)). For example, 50% sequence identity based on “local alignment” means that in an alignment of the full sequence of two compared sequences of any length, a region of similarity or identity of 100 nucleotides in length has 50% of the bases that are the same in the region of similarity or identity.

Allele: As used herein, “allele” or “allelic variant” refers to a specific genetic variant at defined genomic location or locus. An allelic variant is usually presented at a frequency of 50% (0.5) or 100%, depending on whether the allele is heterozygous or homozygous. For example, germline variants are inherited and usually have a frequency of 0.5 or 1. Somatic variants; however, are acquired variants and usually have a frequency of <0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

Amplify: As used herein, “amplify” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, “barcode” in the context of nucleic acids refers to a nucleic acid molecule having a sequence that can serve as an identifier of the molecule (molecular barcode), identifier of a partition (partition barcode) or an identifier of the sample (sample barcode or sample index). For example, individual “barcode” sequences are typically added to DNA fragments during next-generation sequencing (NGS) library preparation so that each read can be identified and sorted before the final data analysis.

Cell-Free Nucleic Acid: As used herein, “cell-free nucleic acid” refers to nucleic acids not contained within or otherwise bound to a cell. In some embodiments, “cell-free nucleic acid” refers to nucleic acids which are not contained within or otherwise bound to a cell at the point of isolation from the subject. Cell-free nucleic acids can include, for example, all non-encapsulated nucleic acids sourced from a bodily fluid (e.g., blood, plasma, scrum, urine, cerebrospinal fluid (CSF), etc.) from a subject. Cell-free nucleic acids include DNA (cfDNA), RNA (cfRNA), and hybrids thereof, including cfDNA and/or cfRNA derived from genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Some cell-free nucleic acids are released into bodily fluid from cancer cells, e.g., circulating tumor DNA (ctDNA). Others are released from healthy cells. ctDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Cellular Origin: As used herein, “cellular origin” in the context of cell-free nucleic acids means the cell type from which a given cell-free nucleic acid molecule derives or otherwise originates (e.g., via a apoptotic process, a necrotic process, or the like). In certain embodiments, for example, a given cell-free nucleic acid molecule may originate from a tumor cell (e.g., a cancerous pulmonary cell, etc.) or a non-tumor or normal cell (e.g., a non-cancerous pulmonary cell, etc.).

Classifier: As used herein, “classifier” generally refers to algorithm computer code that receives, as input, test data and produces, as output, a classification of the input data as belonging to one or another class (e.g., having a DNA damage repair deficiency (DDRD) or not having DDRD, tumor DNA or non-tumor DNA).

Contiguous Sequence: As used herein, “contiguous sequence” or “contig” refers to a set of consecutive nucleic acid bases (that can be overlapping), that together represent a consensus of a nucleic acid region. It can refer to any contiguous stretch of sequence data of sequence reads, or reference intervals, or constructs from reads and/or reference segments (such as assembled reads).

Copy Number Variant: As used herein, “copy number variant,” “CNV,” or “copy number variation” refers to a phenomenon in which sections of the genome are repeated and the number of repeats in the genome varies between individuals in the population under consideration.

Coverage: As used herein, the terms “coverage”, “total molecule count” or “total allele count” are used interchangeably. They refer to the total number of nucleic acid molecules at a particular genomic position in a given sample.

Deoxyribonucleic Acid or Ribonucleic Acid: As used herein, “deoxyribonucleic acid” or “DNA” refers a natural or modified nucleotide which has a hydrogen group at the 2′-position of the sugar moiety. DNA typically includes a chain of nucleotides comprising deoxyribonucleosides that comprise one of four types of nucleobases, namely, adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA typically includes a chain of nucleotides comprising ribonucleosides that comprise one of four types of nucleobases, namely, A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “sequence information,” “nucleic acid sequence,” “nucleotide sequence”, “genomic sequence,” “genetic sequence,” or “fragment sequence,” or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Detect: As used herein, “detect,” “detecting,” or “detection” refers to an act of determining the existence or presence of one or more target nucleic acids (e.g., nucleic acids having targeted mutations or other markers) in a sample.

Enriched Sample: As used herein, “enriched sample” refers to a sample that has been enriched for specific regions of interest. The sample can be enriched by amplifying regions of interest or by using single-stranded DNA/RNA probes or double stranded DNA probes that can hybridize to nucleic acid molecules of interest (e.g., SureSelect® probes, Agilent Technologies). In some embodiments, an enriched sample refers to a subset or portion of the processed sample that is enriched, where the subset or portion of the processed sample being enriched contains nucleic acid molecules from a sample of cell-free polynucleotides or polynucleotides.

Gene: As used herein, “gene” refers to any segment of DNA associated with a biological function. Thus, genes include coding sequences and optionally, the regulatory sequences required for their expression. Genes also optionally include non-expressed DNA segments that, for example, form recognition sequences for other proteins.

Genomic Region: As used herein, “genomic region” means a fixed position on, or section of, a chromosome, such as the position of a gene or a genomic marker. Exemplary genomic markers include transcriptional factor binding regions (e.g., CTCF binding regions, etc.), distal regulatory elements (DREs), repetitive elements (e.g., microsatellites, etc.), intron-exon or exon-intron junctions, transcriptional start sites (TSSs), and the like.

Indel: As used herein, “indel” refers to mutation that involves the insertion or deletion of nucleotide positions in the genome of a subject.

k-mer: As used herein, “k-mer” refers to a string (or sub-string) of length k contained within a genomic sequence. In some embodiments, the length k can be 100 bp or less, 150 bp or less, 200 bp or less or 250 bp or less.

Match: As used herein, “match” means that at least a first value or element is at least approximately equal to at least a second value or element. In certain embodiments, for example, the cellular origin of at least the subset of the DNA molecules from a cfDNA sample is determined when there is at least a substantial or approximate match between a test sample distribution of cfDNA fragment properties and a reference sample distribution of cfDNA fragment properties.

Mutation: As used herein, “mutation,” “nucleic acid variant,” “variant,” “genetic aberration,” or “genetic lesions” refers to a variation from a known reference sequence and includes mutations such as, for example, single nucleotide variants (SNVs), copy number variants or variations (CNVs)/aberrations, insertions or deletions (indels), truncation, gene fusions, transversions, translocations, frame shifts, duplications, repeat expansions, and epigenetic variants. A mutation can be a germline or somatic mutation. In some embodiments, a reference sequence for purposes of comparison is a wildtype genomic sequence of the species of the subject providing a test sample, typically the human genome. In certain cases, a mutation or variant is a “tumor-related genetic variant” that causes or at least contributes to oncogenesis. A driver mutation can be a mutation associated with cancer or another abnormal biological condition. The presence of a driver mutation can be indicative of cancer diagnosis, stratification of a subject to a subtype, tumor burden, tumor in a tissue or organ, tumor metastasis, therapeutic efficacy, or resistance to treatment.

Next Generation Sequencing: As used herein, “next generation sequencing” or “NGS” refers to massively parallel sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization.

Nucleic Acid Tag: As used herein, “nucleic acid tag” refers to a short nucleic acid (e.g., less than about 500, about 100, about 50 or about 10 nucleotides in length), used to label nucleic acid molecules to distinguish nucleic acids from different samples (e.g., representing a sample index), or different nucleic acid molecules in the same sample (e.g., representing a molecular tag), of different types, or which have undergone different processing. Nucleic acid tags can be single stranded, double stranded or at least partially double stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or both ends of the other nucleic acids (e.g., sample nucleic acids to be amplified and/or sequenced). Nucleic acid tags can be decoded to reveal information such as the sample of origin, form or processing of a given nucleic acid. Nucleic acid tags can also be used to enable pooling and/or parallel processing of multiple samples comprising nucleic acids bearing different nucleic acid tags and/or sample indexes in which the nucleic acids are subsequently being deconvoluted by reading the nucleic acid tags. Nucleic acid tags can also be referred to as molecular identifiers or tags, sample identifiers, index tags, and/or barcodes. Additionally or alternatively, nucleic acid tags can be used to distinguish different molecules in the same sample. This includes, for example, uniquely tagging different nucleic acid molecules in a given sample, or non-uniquely tagging such molecules. In the case of non-unique tagging applications, tags with a limited number of different sequences may be used to tag nucleic acid molecules such that different molecules can be distinguished based on, for example, start and/or stop positions where they map to a selected reference genome in combination with at least one nucleic acid tag. Typically, a sufficient number of different nucleic acid tags are used such that there is a low probability (e.g., less than about a 10%, less than about a 5%, less than about a 1%, or less than about a 0.1% chance) that any two molecules will have the same start/stop positions and also have the same nucleic acid tag. Some nucleic acid tags include multiple molecular identifiers to label samples, forms of nucleic acid molecules within a sample, and nucleic acid molecules within a form having the same start and stop positions. Such nucleic acid tags can be referenced using the exemplary form “Ali” in which the uppercase letter indicates a sample type, the Arabic numeral indicates a form of molecule within a sample, and the lowercase Roman numeral indicates a molecule within a form.

Polynucleotide: As used herein, “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases, as is standard in the art.

Prevalence: As used herein, “prevalence” in the context of nucleic acid variants refers to the degree, pervasiveness, or frequency with which a given nucleic acid variant is or was observed in a given sample (e.g., a given bodily fluid sample, a given non-bodily fluid sample, etc.) or other population (e.g., a given population of bodily fluid samples, a given population of non-bodily fluid samples, etc.).

Reference Sample: As used herein, “reference sample” or “reference cfDNA sample” refers a sample of known composition and/or having or known to have or lack specific properties (e.g., known nucleic acid variant(s), known cellular origin, known tumor fraction, known coverage, and/or the like) that is analyzed along with or compared to test samples in order to evaluate the accuracy of an analytical procedure. A reference sample data set typically includes from at least about 25 to at least about 30,000 or more reference samples. In some embodiments, the reference sample data set includes about 50, 75, 100, 150, 200, 300, 400, 500, 600, 700, 800, 900, 1,000, 2,500, 5,000, 7,500, 10,000, 15,000, 20,000, 25,000, 50,000, 100,000, 1,000,000, or more reference samples.

Reference Sequence: As used herein, “reference sequence” or “reference genome” refers to a known sequence used for purposes of comparison with experimentally determined sequences. For example, a known sequence can be an entire genome, a chromosome, or any segment thereof. A reference sequence typically includes at least about 20, at least about 50, at least about 100, at least about 200, at least about 250, at least about 300, at least about 350, at least about 400, at least about 450, at least about 500, at least about 1000, at least about 10,000, at least about 100,000, at least about 1,000,000, at least about 10,000,000, at least about 100,000,000, at least about 1,000,000,000, or more nucleotides. A reference sequence can align with a single contiguous sequence of a genome or chromosome or can include non-contiguous segments that align with different regions of a genome or chromosome. Exemplary reference sequences, include, for example, human genomes, such as, hG19 and hG38.

Sample: As used herein, “sample” means any biological sample capable of being analyzed by the methods and/or systems disclosed herein. In certain aspects of the present disclosure, samples are bodily fluid samples, for example, whole blood or fractions thereof, lymphatic fluid, urine, and/or cerebrospinal fluid, among other bodily fluid types from which cell-free (circulating, not contained within, or otherwise bound to a cell) nucleic acids are sourced. In certain implementations, bodily fluid samples are plasma samples, which are the fluid portions of whole blood exclusive of cells, such as red and white blood cells. In some implementations, bodily fluid samples are serum samples, that is, plasma lacking fibrinogen. In some aspects of the present disclosure, samples are “non-bodily fluid samples” or “non-plasma samples,” that is, biological samples other than “bodily fluid samples” such as, as cellular and/or tissue samples, from which nucleic acids other than cell-free nucleic acids are sourced. In some embodiments, the sample can include body tissue or tissue biopsy.

Sensitivity: As used herein, “sensitivity” in the context of a given assay or method refers to the ability of the assay or method to detect and distinguish between targeted (e.g., nucleic acid variants) and non-targeted analytes.

Sequence fragment: As used herein, “sequence fragmentConstructing the allele k-mer data structure may comprise dividing the known allele sequences into a quantity of k-mers. For example, a quantity of k-mers having a length from about 100 nucleotides to about 200 nucleotides. In an embodiment, the quantity of k-mers may have a length of 125-175 nucleotides, 130-160 nucleotides, 135-155 nucleotides, 140-150 nucleotides. In an embodiment, the quantity of k-mers may have a length of 140, 141, 142, 143, 144, or 145 nucleotides. Constructing the allele k-mer data structure may comprise associating each k-mer with metadata. The metadata may comprise, for example, an indication of a quantity of alleles that contain the k-mer and, for each allele that contains the k-mer, an allele identifier and a start position of the k-mer.

Sequence read: As used herein, “read,” “sequence read,” or “sequencing read” refers to the sequence of base pairs corresponding to all or a part of a sequence fragment.

Sequencing: As used herein, “sequencing” refers to any of a number of technologies used to determine the sequence (e.g., the identity and order of monomer units) of a biomolecule, e.g., a nucleic acid such as DNA or RNA. Exemplary sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLID™ sequencing, MS-PET sequencing, and a combination thereof. In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

Sequence Information: As used herein, “sequence information” in the context of a nucleic acid polymer means the order and/or identity of monomer units (e.g., nucleotides, etc.) in that polymer.

Single Nucleotide Variant: As used herein, “single nucleotide variant” or “SNV” means a mutation or variation in a single nucleotide that occurs at a specific position in a genome or a given genomic sequence.

Specificity: As used herein, “specificity” in the context of a diagnostic analysis or assay refers to the extent to which the analysis or assay detects an intended target analyte to the exclusion of other components of a given sample.

Status: As used herein, “status” in the context of subjects refers to one or more states of a given subject, such as whether or not the subject has cancer.

Subject: As used herein, “subject” or “test subject” refers to an animal, such as a mammalian species (e.g., human) or avian (e.g., bird) species, or other organism, such as a plant. More specifically, a subject can be a vertebrate, e.g., a mammal such as a mouse, a primate, a simian, or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” In some embodiments, the subject is a human who has, or is suspected of having cancer. For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed with or suspected of having a disease, e.g., a cancer, an auto-immune disease. A “reference subject” refers to a subject known to have or lack specific properties (e.g., known cancer or disease status, known nucleic acid variant(s), known cellular origin, known tumor fraction, known coverage, and/or the like).

Threshold: As used herein, “threshold” refers to a separately determined value used to characterize or classify experimentally determined values. In certain embodiments, for example, “threshold value” refers to a selected value to which a quantitative value is compared in order to determine that a given target nucleic acid variant is absent at a given genetic locus.

Value: As used herein, “value” or “score” generally refers to an entry in a data set can be anything that characterizes the feature to which the value refers. This includes, without limitation, numbers, words or phrases, symbols (e.g., + or −) or degrees.

### DETAILED DESCRIPTION

### II. INTRODUCTION

Provided herein are methods and systems for determining an allele type from a nucleic acid sample obtained from a test subject. In some aspects, the nucleic acid sample can be, but is not limited to, cell-free nucleic acid (cfNA), genomic DNA, or RNA. In an embodiment, the nucleic acid sample may be derived from a specific chromosome and/or from a specific region of a chromosome. In an embodiment, the nucleic acid sample may be derived from all or a portion of the major histocompatibility complex (MHC) region of human chromosome 6. In an embodiment, the nucleic acid sample may be derived from all or a portion of the killer cell immunoglobulin-like receptor (KIR) region of human chromosome 19. In an embodiment, the nucleic acid sample may be derived in whole or in part from both the MHC region of human chromosome 6 and the KIR region of human chromosome 19.

a. Major Histocompatibility Complex (MHC)

The MHC is a set of cell surface molecules encoded by a large gene family which controls a major part of the immune system in all vertebrates. The major function of major histocompatibility complexes is to bind to peptide fragments derived from pathogens and display them on the cell surface for recognition by the appropriate T-cells.

The MHC contains two types of polymorphic MHC genes, the class I and class II genes, which encode two groups of structurally distinct but homologous proteins, and other nonpolymorphic genes whose products are involved in antigen presentation.

The human MHC is called human leukocyte antigen (HLA). HLA proteins are encoded by genes of the MHC. HLA class I antigens include HLA-A, HLA-B, and HLA-C. HLA class II antigens include HLA-DR, HLA-DQ, HLA-DP, HLA-DM, HLA-DOA, HLA-DOB. HLA's corresponding to MHC class I (A, B, and C) present peptides from inside the cell. HLA's corresponding to MHC class II (DP, DM, DOA, DOB, DQ, and DR) present antigens from outside of the cell to T-lymphocytes. MHC molecules mediate the binding of a given T cell receptor to a given antigen, so MHC polymorphism across individuals modulates the T cell response to a given antigen. Clinical applications of HLA typing include vaccine trials, disease associations, adverse drug reactions, platelet transfusion, and transplantation of organs and stem cells.

There are numerous alleles at each HLA gene locus (A1, A2, A3, etc.). Each person inherits one complete set of HLA alleles (haplotype) from each parent, and this combination of encoded proteins constitutes a person's HLA type (e.g., different antigens of A23, A31, B7, B44, C7, C8, DR4, DR7, DQ2, DQ7, DP2, DP3). There are more than 50,000 different known HLA types.

Certain diseases, especially of autoimmune nature, are associated with particular HLA types. The association level varies among diseases, and there is generally a lack of a strong concordance between the HLA type and the disease. The exact mechanisms underlying the most HLA-disease association are not well understood, and other genetic and environmental factors may play roles as well. Among the most prominent associations are narcolepsy with HLA-DQB1*0602/HLA-DRB1*1501, ankylosing spondylitis with HLA-B27, and celiac disease with HLA-DQB1*02. The HLA-A1, B8, DR17 haplotype is frequently associated with autoimmune disorders. Rheumatoid arthritis is associated with a particular sequence of the amino acid positions 66 to 75 in the DRβ1 chain that is common to the major subtypes of HLA-DR4 and DR1. Type I diabetes mellitus is associated with HLA-DR3,4 heterozygotes, and the absence of asparagine at position 57 on the DQβ1 chain appears to render susceptibility to this disease.

HLA-A, HLA-B, and HLA-DR have long been known as major transplantation antigens. Recent clinical data indicate that HLA-C matching also affects the clinical outcomes of hematopoietic stem cell transplantation. Allogeneic hematopoietic stem cell transplantation is used to treat hematologic malignancy, severe aplastic anemia, severe congenital immunodeficiencies, and selected inherited metabolic diseases. The HLA system is the major histocompatibility barrier in stem cell transplantation, and the degree of HLA matching is predictive of the clinical outcome. HLA mismatch between a recipient and a stem cell donor represents a risk factor for graft rejection/failure and acute graft-versus-host disease (GVHD). The latter is caused by immunocompetent donor T cells contained in the stem cell products. T-cell depletion of donor marrow results in lower incidence of acute GVHD, but higher incidence of graft failure, graft rejection, malignant disease relapse (i.e., loss of the graft-versus-leukemia effect), impaired immune recovery, and later complication of Epstein-Barr virus-associated lymphoproliferative disorders.

b. Killer Cell Immunoglobulin-Like Receptor (KIR)

Natural Killer (NK) cells are part of the innate immune system and are specialized for early defense against infection as well as tumors. The NK cells were first discovered as a result of their ability to kill tumor cell targets. Unlike cytolytic T-cells, NK cells can kill targets in a non-major histocompatibility complex (non-MHC)-restricted manner. As an important part of the innate immune system, the NK cells comprise about 10% of the total circulating lymphocytes in the human body.

Because of their ability to kill other cells, NK cells are normally kept under tight control. All normal cells in the body express the MHC class I molecules on their surface. These molecules protect normal cells from killing by the NK cells because they serve as ligands for many of the receptors found on NK cells. Cells lacking sufficient MHC class I on their surface are recognized as “abnormal” by NK cells and killed. Simultaneously with killing the abnormal cells, the NK cells also elicit a cytokine response.

Natural killer cells constitute a rapid-response force against cancer and viral infections. These specialized white blood cells originate in the bone marrow, circulate in the blood, and concentrate in the spleen and other lymphoid tissues. NK cells key their activities on a subset of the HLA proteins that occur on the surfaces of healthy cells but that virus- and cancer-weakened cells shed. When NK cells encounter cells that lack HLA proteins, they attack and destroy them-thus preventing the cells from further spreading the virus or cancer. NK cells are distinguished from other immune system cells by the promptness and breadth of their protective response. Other white blood cells come into play more slowly and target specific pathogens—cancers, viruses, or bacteria—rather than damaged cells in general.

The natural killer cell immunoglobulin-like receptor (KIR) gene family is one of several families of receptors that encode important proteins found on the surface of NK cells. A subset of the KIR genes, namely the inhibitory KIR, interact with the HLA class I molecules, which are encoded within the human MHC. Such interactions allow communication between the NK cells and other cells of the body, including normal, virally infected, or cancerous cells. This communication between KIR molecules on the NK cells and HLA class I molecules on all other cells, helps determine whether or not cells in the body are recognized by the NK cells as self or non-self. Cells which are deemed to be ‘non-self’ are targeted for killing by the NK cells.

The KIR gene family consists of 16 genes (KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1/S1, KIR3DL2, KIR3DL3, KIR2 DP1 and KIR3DP1.) The KIR gene cluster is located within a 100-200 kb region of the Leukocyte Receptor Complex (LRC) located on chromosome 19 (19q13.4) The gene complex is thought to have arisen by gene duplication events occurring after the evolutionary split between mammals and rodents The KIR genes are arranged in a head-to tail fashion, with only 2.4 kb of sequence separating the genes, except for one 14 kb sequence between 3DP1 and 2DL4. Because the KIR genes arose by gene duplication, they are very similar in sequence, showing 90-95% identity with one another. Human individuals differ in the number and type of KIR genes that they inherit; the KIR genotype of individuals and within ethnic groups can be quite different. At the chromosomal level, there are two distinct types of KIR haplotypes (See Martin et al. Immunogenetics. (2008) December; 60(12):767-774). The A-haplotype contains no stimulatory genes (2DS and 3DS1) other than 2DS4, no 2DL5 genes and no 2DL2 genes. The B-haplotype is more variable in gene content, with different B-haplotypes containing different numbers of stimulatory genes, either one or two 2DL5 genes, etc. (Martin M P, et al. (2008) KIR haplotypes defined by segregation analysis in 59 Centre d'Etude Polymorphisme Humain (CEPH) families. Immunogenetics, December; 60(12):767-774.).

All of the KIR proteins are anchored to the cell membrane, with either two or three extracellular immunoglobulin-like domains and a cytoplasmic tail. Nine KIR genes (KIR2DL and KIR3DL) encode proteins with long cytoplasmic tails that contain immune tyrosine-based inhibitory motifs (ITIM). These KIR proteins can send inhibitory signals to the natural killer cell when the extra-cellular domain has come into contact with its ligand. The remaining KIR genes encode proteins with short cytoplasmic tails. These proteins send activating signals via adaptor molecules like DAP12.

KIR receptor structure and the identity of the HLA class I ligands for each KIR receptor are described in Parham P. et al., Alloreactive killer cells: hindrance and help for hematopoietic transplants. Nature Rev. Immunology. (2003)3:108-122. The nomenclature for the killer-cell immunoglobulin-like receptors (KIRs) describes the number of extracellular immunoglobulin-like domains (2D or 3D) and the length of the cytoplasmic tail (L for long, S for short). Each immunoglobulin-like domain is depicted as a loop, each immunoreceptor tyrosine-based inhibitory motif (ITIM) in the cytoplasmic tail as an oblong shape, and each positively charged residue in the transmembrane region as a diamond.

The strength of the interactions between the KIR and their HLA class I ligands can be dependent upon both the KIR sequence and the HLA sequence. While the HLA region has been studied for over 40 years, the KIR molecules were first described (as NKB1) in the mid-1990s (Lanier et al. (1995) The NKB1 and HP-3E4 NK cells receptors are structurally distinct glycoproteins and independently recognize polymorphic HLA-B and HLA-C molecules. J. Immunol. April 1; 154(7):3320-3327 and Litwin V. et al. (1994) NKB1: a natural killer cell receptor involved in the recognition of polymorphic HLA-B molecules. J Exp Med. August 1; 180(2):537-543). The first years of discovery were mainly devoted to describing the different KIR genes, and methods were developed to determine individual KIR genotypes. Utilizing these methods, KIR gene associations with autoimmune disease and recipient survival after allogeneic hematopoietic cell transplantation have been shown (Parham P. (2005) MHC class I molecules and KIRs in human history, health and survival. Nature reviews, March; 5(3):201-214). It is now clear that each KIR gene has more than one sequence; that is, each KIR gene has variable sequence because of single nucleotide polymorphisms (SNPs), and in some instances, insertions or deletions within the coding sequence. Studies have shown that KIR3DL1 polymorphism can affect not only the expression levels of KIR3DL1 on natural killer cells, but also the binding affinity of KIR3DL1 to its ligand.

Studies designed to investigate the role of KIR in human disease have shown an association with various KIR genes and viral infections such as CMV, HCV and HIV, autoimmune diseases, cancer and preeclampsia (Parham P. (2005) MHC class I molecules and KIRs in human history, health and survival. Nature reviews, March; 5(3):201-214). In a recent study on genetic susceptibility to Crohn's disease, an inflammatory autoimmune bowel disease, it was found that patients who are heterozygous for KIR2DL2 and KIR2DL3 and homozygous for the C2 ligand are susceptible to disease, whereas the C1 ligand is protective. (Hollenbach J A et al. (2009) Susceptibility to Crohn's Disease is mediated by KIR2DL2/KIR2DL3 heterozygosity and the HLAC ligand. Immunogenetics. October; 61(10):663-71). Other studies on KIR and unrelated hematopoietic cell transplantation (HCT) for Acute Myeloid Leukemia (AML) have shown a significantly higher 3 year overall survival rate and a 30% overall improvement in the risk of relapse-free survival with B/x donors compared to A/A donors. (Cooley S, et al. (2009) Donors with group B KIR haplotypes improve relapse-free survival after unrelated hematopoietic cell transplantation for acute myelogenous leukemia. Blood. January 15; 113(3):726-732; and Miller J S, et al. (2007) Missing KIR-ligands is associated with less relapse and increased graft versus host disease (GVHD) following unrelated donor allogeneic HCT. Blood, 109(11):5058-5061). Such studies have been performed with knowledge of the KIR genotype of patients and controls, but have not been performed for KIR at an allelic level. Much as specific HLA alleles have been shown to be important in human disease, (for example HLA-DR3 and HLA-DR4 association with type I diabetes and HLA-DR8 with juvenile rheumatoid arthritis) the ability to genotype KIR at the allelic level will refine studies associating KIR with human disease.

In an embodiment, the methods and systems disclosed may be applied to a nucleic acid sample derived from one or more genes of the MHC region of human chromosome 6. In an embodiment, the nucleic acid sample may be derived from one or more genes of the KIR region of human chromosome 19. In an embodiment, the nucleic acid sample may be derived in whole or in part may be derived from one or more genes of the MHC region of human chromosome 6 and from one or more genes of the KIR region of human chromosome 19. Essentially any number of genes may be evaluated using the methods and related aspects of the present disclosure. In some embodiments, for example, sets of genes targeted for analysis, as described herein, include at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 100, 1,000, 10,000, or more genes. A non-exhaustive list of genes, one or more of which are optionally selected for evaluation using the methods and related aspects disclosed herein is provided in Table 1.

TABLE 1

| | | | |
|---|---|---|---|
| HLA-A | HLA-X | HLA-DQB3 | MICC |
| HLA-B | HLA-Y | HLA-DOA | MICD |
| HLA-C | HLA-Z | HLA-DOB | MICE |
| HLA-E | HLA-DRA | HLA-DMA | KIR2DL1 |
| HLA-F | HLA-DRB1 | HLA-DMB | KIR2DL2/L3 |
| HLA-G | HLA-DRB2 | HLA-DPA1 | KIR2DL4 |
| HLA-H | HLA-DRB3 | HLA-DPB1 | KIR2DL5A |
| HLA-J | HLA-DRB4 | HLA-DPA2 | KIR2DL5B |
| HLA-K | HLA-DRB5 | HLA-DPB2 | KIR2DS1 |
| HLA-L | HLA-DRB6 | HLA-DPA3 | KIR2DS2 |
| HLA-N | HLA-DRB7 | HFE | KIR2DS3 |
| HLA-P | HLA-DRB8 | TAP1 | KIR2DS4 |
| HLA-S | HLA-DRB9 | TAP2 | KIR2DS5 |
| HLA-T | HLA-DQA1 | PSMB9 | KIR3DL1/S1 |
| HLA-U | HLA-DQB1 | PSMB8 | KIR3DL2 |
| HLA-V | HLA-DQA2 | MICA | KIR3DL3 |
| HLA-W | HLA-DQB2 | MICB | |

### III. EXEMPLARY SYSTEMS AND METHODS

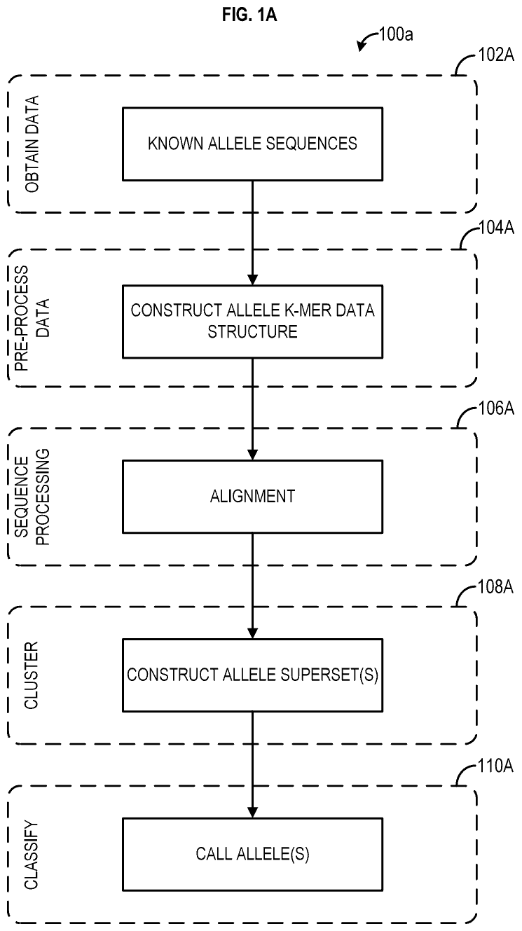

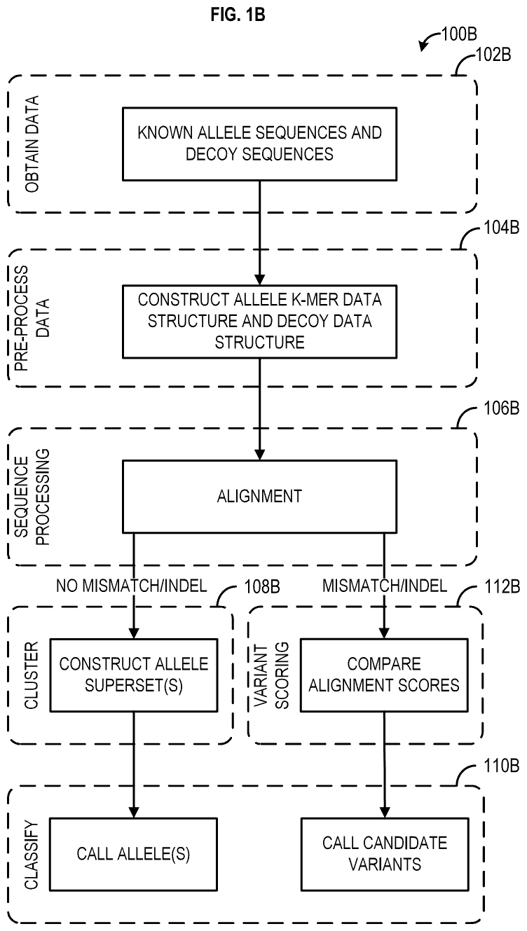

FIG. 1A is a flow chart that schematically depicts an example technique for allele typing in a cell-free nucleic acid (cfDNA) sample obtained from a test subject. Allele typing may be used to determine one or more alleles present at a locus of a chromosome. As shown, a method 100A, at step 102A, may comprise obtaining data. The data may comprise sequence data, such as allele sequence data. For example, step 102A may comprise obtaining (or otherwise determining, retrieving, receiving, etc.) known allele sequences. For example, known allele sequences may be downloaded from the IPD-IMGT/HLA Database and/or the IPD-KIR Database. By way of example, known HLA allele sequences may be downloaded from ftp.ebi.ac.uk/pub/databases/ipd/imgt/hla and known KIR allele sequences may be downloaded from ftp.ebi.ac.uk/pub/databases/ipd/kir.

The known allele sequences may be any type of allele sequence. In an embodiment, the known allele sequences may be known human leukocyte antigen (HLA) allele sequences. In an embodiment, the known allele sequences may be known killer cell immunoglobulin-like receptor (KIR) allele sequences.

At step 104A, the data may be pre-processed. For example, step 104A may comprise constructing an allele k-mer data structure. The allele k-mer data structure may be a database. The allele k-mer data structure may be a flat file. The allele k-mer data structure may be any form of data structure. Constructing the allele k-mer data structure may comprise dividing the known allele sequences into a quantity of k-mers. For example, a quantity of k-mers having a length from about 100 nucleotides to about 200 nucleotides. In an embodiment, the quantity of k-mers may have a length of 143 nucleotides. Constructing the allele k-mer data structure may comprise associating each k-mer with metadata. The metadata may comprise, for example, an indication of a quantity of alleles that contain the k-mer and, for each allele that contains the k-mer, an allele identifier and a start position of the k-mer.

At step 106A, sequence processing may be performed. For example, step 106A may comprise obtaining (or otherwise determining, retrieving, receiving, etc.) sequence read pairs (e.g., test sequence reads) from a cell-free nucleic acid (cfDNA) sample obtained from a test subject. Step 106A may comprise performing an alignment between the test sequence reads and the known allele sequences. For example, step 106A may comprise performing an alignment between the test sequence reads and the k-mers in the allele k-mer data structure. The sequence processing may determine an allele(s) supported by a test sequence read(s). An allele may be supported by more than one test sequence read. A test sequence read may support more than one allele. In an embodiment, a test sequence read may be found to support an allele if the test sequence read aligns to the allele (e.g., a k-mer of the allele) with over a threshold percent identity. For example, the threshold percent identity may be, 80%, 85%, 90%, 95%, 96%, 97%, 98%, 99%, 100%, and the like. In an embodiment, the threshold percent identity may be 100%, requiring a “perfect” match between a test sequence read and the allele (e.g., a k-mer of the allele).

In an embodiment, step 106A may comprise determining a number of test sequence read families that support an allele(s) (e.g., a number of nucleic acid molecules that support an allele(s)). Each test sequence read may comprise a barcode. The barcode may identify the nucleic acid molecule (e.g., test sequence read family) with which the test sequence read is associated. In an embodiment, a test sequence read family may be found to support an allele if the test sequence read family aligns to the allele with over a threshold percent identity. For example, the threshold percent identity may be, 80%, 85%, 90%, 95%, 96%, 97%, 98%, 99%, 100%, and the like. In an embodiment, the threshold percent identity may be 100%, requiring a “perfect” match between a test sequence read family and the allele (e.g., a k-mer of the allele).

At step 108A, a clustering operation may be performed. The alleles may be sorted by the number of supporting test sequence reads (or by the number of supporting test sequence read families) and one or more allele supersets may be constructed. An allele superset may be constructed by determining a first allele associated with a highest number of supporting test sequence reads (or associated with a highest number of supporting test sequence read families). The first allele may form the basis of an allele superset. Additional alleles may be added to the allele superset if a given allele is associated with supporting test sequence reads (or supporting test sequence read families) are themselves a subset of the supporting test sequence reads (or supporting test sequence read families) of the first allele. Alleles that are not incorporated into the allele superset of the first allele may be used to construct one or more additional allele supersets in a similar fashion.

An allele superset may be a data structure. An allele superset may be a database. An allele superset may be a flat file. An allele superset may comprise a representation of a Hasse diagram. A Hasse diagram is a representation of the relation of elements of a partially ordered set with an implied upward orientation. A point, or node, may represent each element of the partially ordered set and nodes may be joined with a line segment according to the following rules: 1) if p<q in the partially ordered set, then the point corresponding to p appears lower in the drawing than the point corresponding to q; 2) the two points p and q will be joined by a line segment if p is related to q. In an embodiment, the Hasse diagram may be represented as a graph data structure, such as a directed acyclic graph (DAG) and/or the like. For example, a DAG comprising a line from node A to node B if node A strictly contains node B and there is no node C such that node A strictly contains node C and node C strictly contains node B.

At step 110A, an allele may be classified. For example, at step 110A an allele type may be determined for a given allele. The allele may be classified based on the one or more allele supersets. In an embodiment, in the event only one allele superset is constructed the first allele of the superset may be classified as the allele present at the locus (e.g., haploid locus) of the chromosome. In an embodiment, in the event that a plurality of allele supersets are constructed, the first alleles of the two supersets having a cumulative largest number of distinct supporting test sequence reads (or a cumulative largest number of distinct supporting test sequence read families) may be classified as the alleles present at the locus (e.g., diploid locus) of the chromosome.

The classification of the allele(s) may be used to direct treatment of a subject. It may have been previously unknown whether the subject has a disease or it may be known that the subject has a disease. The disease may be cancer. The methods may comprise administering one or more therapies to the subject to treat the disease. The therapies may comprise administering immunotherapy, administering chemotherapy, administering radiation therapy, or performing surgery to resect all or a portion of the tumor. The methods may comprise assisting in a communication of determination of the classification of the allele(s) to a subject associated with the test sample.

In an embodiments, the method 100A may be performed using known HLA allele sequences to determine the subject's HLA genotype and/or the method 100A may be performed using known KIR allele sequences to determine the subject's KIR genotype.

FIG. 1B is a flow chart that schematically depicts an example technique for allele typing and/or variant calling in a cell-free nucleic acid (cfDNA) sample obtained from a test subject. Allele typing may be used to determine one or more alleles present at a locus of a chromosome. Variant calling may be used to identify the presence of a known, or unknown variant. Variant calling may be used to characterize cancer progression. As shown, a method 100B, at step 102B, may comprise obtaining data. The data may comprise sequence data, such as allele sequence data and/or decoy sequence data.

For example, step 102B may comprise obtaining (or otherwise determining, retrieving, receiving, etc.) known allele sequences. For example, known allele sequences may be downloaded from the IPD-IMGT/HLA Database and/or the IPD-KIR Database. By way of example, known HLA allele sequences may be downloaded from ftp.ebi.ac.uk/pub/databases/ipd/imgt/hla and known KIR allele sequences may be downloaded from ftp.ebi.ac.uk/pub/databases/ipd/kir. The known allele sequences may be any type of allele sequence. In an embodiment, the known allele sequences may be known human leukocyte antigen (HLA) allele sequences. In an embodiment, the known allele sequences may be known killer cell immunoglobulin-like receptor (KIR) allele sequences.

For example, step 102B may comprise obtaining (or otherwise determining, retrieving, receiving, etc.) decoy sequences. The decoy sequences may be any type of decoy sequence. In an embodiment, the decoy sequences may be one or more of, “alt” sequences, unprobed known HLA allele sequences, probed known HLA allele sequences (to address small germline inaccuracies, 3 vs 4 sets of digits, combinations thereof, and the like. The decoy sequences are sequences of genomic material (human, in general) similar to the sequences we want to look at (for example, the regions we want to genotype). These are not already part of the reference because they encode an alternate form of a region or gene (hence the name “alt”). The problem for us is that we deploy targeted sequencing, which is a way to select only molecules from portions of genome matching some specified region (these “specified regions” are called probes, or baits, and in our case are 120 bases long): what happens is that sometimes a probe designed to capture molecules from a region of interest, instead captures molecules from one of these “alt” sequences. We can detect this because in these cases the read (or read pair) aligns better on the decoy than on the human reference.

In an embodiment, the decoy sequences may comprise decoy sequences selected to identify contamination in the test sample. The one or more decoy sequences may comprise one or more non-human reference sequences. For example, the one or more decoy sequences may comprise bovine reference sequences, rat reference sequences, microbial reference sequences, combinations thereof and the like. Any test sequences pairs aligning to a non-human decoy sequence may be used to support a conclusion that the test sample has been contaminated with DNA from sources other than the test subject. The idea is the same as above, only we use here as “decoy” the sequence of our suspected contaminants. For example, assume we suspect there is some cow DNA in our sample (which happened!), then we add the whole cow genome to our decoy list, and when we align a read pair to both the human reference and to the cow genome (the decoy), we find that the contaminant molecules align better on cow than on human.

At step 104B, the data may be pre-processed. For example, step 104B may comprise constructing an allele k-mer data structure. The allele k-mer data structure may be a database. The allele k-mer data structure may be a flat file. The allele k-mer data structure may be any form of data structure. Constructing the allele k-mer data structure may comprise dividing the known allele sequences into a quantity of k-mers. For example, a quantity of k-mers having a length from about 100 nucleotides to about 200 nucleotides. In an embodiment, the quantity of k-mers may have a length of 143 nucleotides. Constructing the allele k-mer data structure may comprise associating each k-mer with metadata. The metadata may comprise, for example, an indication of a quantity of alleles that contain the k-mer and, for each allele that contains the k-mer, an allele identifier and a start position of the k-mer.

For example, step 104B may comprise constructing a decoy data structure. The decoy data structure may be a database. The decoy data structure may be a flat file. The decoy data structure may be any form of data structure. Structuring the algorithm, like this (ie, with a target sequence plus decoy sequence) allows us to keep some flexibility. The idea is that we can always add to the decoy any number of as-yet unknown “problematic” sequence, where in this case problematic means sequence similar to the one of our targets (in other words, sequence we could accidentally pick-up with our targeted sequencing tech dev, instead of the target region).

At step 106B, sequence processing may be performed. For example, step 106B may comprise obtaining (or otherwise determining, retrieving, receiving, etc.) sequence reads (e.g., test sequence reads) from a cell-free nucleic acid (cfDNA) sample obtained from a test subject. Step 106B may comprise performing an alignment between the test sequence reads and the known allele sequences. For example, step 106B may comprise performing an alignment between the test sequence reads and the k-mers in the allele k-mer data structure. The sequence processing may determine an allele(s) supported by a test sequence read(s). An allele may be supported by more than one test sequence read. A test sequence read may support more than one allele. In an embodiment, a test sequence read may be found to support an allele if the test sequence read aligns to the allele (e.g., a k-mer of the allele) with over a threshold percent identity. For example, the threshold percent identity may be, 80%, 85%, 90%, 95%, 96%, 97%, 98%, 99%, 100%, and the like. In an embodiment, the threshold percent identity may be 100%, requiring a “perfect” match between a test sequence read and the allele (e.g., a k-mer of the allele) indicating no mismatches and no indels. In an embodiment, the threshold percent identity may be less than 100%, requiring an “imperfect” match between a test sequence read and the allele (e.g., a k-mer of the allele) indicating at least one mismatch and/or at least one indel. An indication of percent identity may be determined for each alignment and stored for later processing. The results of an alignment may be represented by an alignment score, described in further detail with regard to the alignment component 215. The alignment score may equal the sum of the number of mismatches and the number of indels.

In an embodiment, step 106B may comprise determining a number of test sequence read families that support an allele(s) (e.g., a number of nucleic acid molecules that support an allele(s)). Each test sequence read may comprise a barcode. The barcode may identify the nucleic acid molecule (e.g., test sequence read family) with which the test sequence read is associated. In an embodiment, a test sequence read family may be found to support an allele if the test sequence read family aligns to the allele with over a threshold percent identity. For example, the threshold percent identity may be, 80%, 85%, 90%, 95%, 96%, 97%, 98%, 99%, 100%, and the like. In an embodiment, the threshold percent identity may be 100%, requiring a “perfect” match between a test sequence read family and the allele (e.g., a k-mer of the allele).

Step 106B may comprise performing an alignment between the test sequence reads and the decoy sequences. For example, step 106B may comprise performing an alignment between the test sequence reads and the decoy sequences in the decoy data structure. The sequence processing may determine a decoy sequence(s) supported by a test sequence read(s). In an embodiment, a test sequence read may be found to support a decoy sequence if the test sequence read aligns to the decoy sequence with over a threshold percent identity. For example, the threshold percent identity may be, 80%, 85%, 90%, 95%, 96%, 97%, 98%, 99%, 100%, and the like. In an embodiment, the threshold percent identity may be 100%, requiring a “perfect” match between a test sequence read and the decoy sequence) indicating no mismatches and no indels. An indication of percent identity may be determined for each alignment and stored for later processing. In an embodiment, one or more test sequence reads that align to one or more decoy sequences with 100% identity may be discarded and not used for further processing. In an embodiment, to the extent the decoy sequences comprise one or more non-human sequences, any test sequence reads that match to a non-human decoy sequence with 100% identify may be used to support identification of the test sample as being contaminated. A notification associated with potential contamination may be generated and/or sent.

The results of an alignment may be represented by an alignment score, described in further detail with regard to the alignment component 215. The alignment score may equal the sum of the number of mismatches and the number of indels.

At step 108B, a clustering operation may be performed based on alignments between the test sequence reads and the known allele sequences. The known alleles may be sorted by the number of supporting test sequence reads (or by the number of supporting test sequence read families) and one or more allele supersets may be constructed. An allele superset may be constructed by determining a first allele associated with a highest number of supporting test sequence reads (or associated with a highest number of supporting test sequence read families). The first allele may form the basis of an allele superset. Additional alleles may be added to the allele superset if a given allele is associated with supporting test sequence reads (or supporting test sequence read families) are themselves a subset of the supporting test sequence reads (or supporting test sequence read families) of the first allele. Alleles that are not incorporated into the allele superset of the first allele may be used to construct one or more additional allele supersets in a similar fashion.

An allele superset may be a data structure. An allele superset may be a database. An allele superset may be a flat file. An allele superset may comprise a representation of a Hasse diagram. A Hasse diagram is a representation of the relation of elements of a partially ordered set with an implied upward orientation. A point, or node, may represent each element of the partially ordered set and nodes may be joined with a line segment according to the following rules: 1) if p<q in the partially ordered set, then the point corresponding to p appears lower in the drawing than the point corresponding to q; 2) the two points p and q will be joined by a line segment if p is related to q. In an embodiment, the Hasse diagram may be represented as a graph data structure, such as a directed acyclic graph (DAG) and/or the like. For example, a DAG comprising a line from node A to node B if node A strictly contains node B and there is no node C such that node A strictly contains node C and node C strictly contains node B.

At step 110B, an allele may be classified. For example, at step 110B an allele type may be determined for a given allele. The allele may be classified based on the one or more allele supersets. In an embodiment, in the event only one allele superset is constructed the first allele of the superset may be classified as the allele present at the locus (e.g., haploid locus) of the chromosome. In an embodiment, in the event that a plurality of allele supersets are constructed, the first alleles of the two supersets having a cumulative largest number of distinct supporting test sequence reads (or a cumulative largest number of distinct supporting test sequence read families) may be classified as the alleles present at the locus (e.g., diploid locus) of the chromosome.

The classification of the allele(s) may be used to direct treatment of a subject. It may have been previously unknown whether the subject has a disease or it may be known that the subject has a disease. The disease may be cancer. The methods may comprise administering one or more therapies to the subject to treat the disease. The therapies may comprise administering immunotherapy, administering chemotherapy, administering radiation therapy, or performing surgery to resect all or a portion of the tumor. The methods may comprise assisting in a communication of determination of the classification of the allele(s) to a subject associated with the test sample.

In an embodiments, the method 100B may be performed using known HLA allele sequences to determine the subject's HLA genotype and/or the method 100B may be performed using known KIR allele sequences to determine the subject's KIR genotype.

At step 112B, alignment scores of remaining test sequence reads that did not align with 100% identify to either a known allele sequence or a decoy sequence may be compared. The remaining test sequence reads may have an alignment score associated with the known allele sequences (e.g., a germline alignment score) and an alignment score associated with the decoy sequences (e.g., a decoy alignment score). A test sequence read pair associated with a germline alignment score that is less than a decoy alignment score may be discarded. A test sequence read pair associated with a germline alignment score that is greater than a decoy alignment score may be sent for variant calling at step 110B. If a test sequence read pair aligns to two or more known allele sequences (e.g., aligns to two or more entries in a fasta file), the first entry in the fasta file with the highest alignment score is selected as the alignment.

At step 110B, the test sequence read pairs associated with a germline alignment score that is greater than a decoy alignment score may be analyzed to determine and/or identify the test sequence read pairs as a variant. Variant calling is the process of identifying true differences between sequence reads of test samples and a reference sequence. Variant calling may be performed as further described with regard to the variant caller component 219 below. In an embodiment, the test sequence read pairs may be identified as a somatic variant. In an embodiment, the test sequence read pairs may be identified as a variant that is a candidate variant associated with a somatic event. At step 110B, candidate variants may be identified in the test sequence read pairs. In one embodiment, the candidate variants may be identified by comparing the test sequence read pairs to a reference sequence of a target region of a reference genome (e.g., human reference genome hg19). Edges of the test sequence read pairs may be aligned to the reference sequence and the genomic positions of mismatched edges and mismatched nucleotide bases adjacent to the edges recorded as the locations of candidate variants. In some embodiments, the genomic positions of mismatched nucleotide bases to the left and right edges are recorded as the locations of called variants. Additionally, candidate variants may be identified based on the sequencing depth of a target region. In particular, more confidence may be obtained in identifying variants in target regions that have greater sequencing depth, for example, because a greater number of sequence reads help to resolve (e.g., using redundancies) mismatches or other base pair variations between sequences.

In an embodiment, the reference sequence used for variant calling may comprise one or more reference sequences. The one or more reference sequences may be selected to identify contamination in the test sample. The one or more reference sequences may comprise one or more non-human reference sequences. For example, the one or more reference sequences may comprise a bovine reference sequences, rat reference sequences, microbial reference sequences, combinations thereof, and the like. Any test sequences pairs identified as a non-human variant may be used to support a conclusion that the test sample has been contaminated with DNA from sources other than the test subject.

Clinical applications of HLA typing and/or variant calling include vaccine trials, disease associations, adverse drug reactions, platelet transfusion, and transplantation of organs and stem cells.

In some embodiments, the methods include determining a subject's HLA type as described herein, and determining that the subject is predisposed to developing rheumatoid arthritis (RA) if it has been determined that one or more of HLA-DRB1*04:01, *04:04, and *04:08; HLA-DRB1*04:05; HLA-DRB1*01:01 and *01:02; HLA-DRB1*14:02; HLA-DRB1*10:01; and/or HLA-DRB1*01:01, *04:01, *04:04, and *04:05, is present.

In some embodiments, the methods include determining a subject's HLA type as described herein, and determining that the subject is predisposed to developing multiple sclerosis (MS) if it has been determined that the HLA-DRB1*15:01, HLA-DQB1*06:02, HLA-DRB1*01:08, HLA-DRB1*03:01, and/or HLA-DRB1*13:03 alleles are present.

In some embodiments, the methods include determining a subject's HLA type as described herein, and determining that the subject is predisposed to developing systemic lupus erythematosus (SLE) if it has been determined that the HLA-DRB1, HLA-DR2 (DRB1*15:01), HLA-DR3 (DRB1*03:01), HLA-DRB1*08:01, and/or HLA-DQA1*01:02 alleles are present.

In some embodiments, the methods include determining a subject's HLA type as described herein, and determining that the subject is predisposed to developing Type 1 diabetes mellitus (TID) if it has been determined that HLA-DQB1*03:02 and/or DQB1*02:01 are present.

In some embodiments, the methods include determining a subject's HLA type as described herein, and determining that the subject is predisposed to developing Sjögren's syndrome (SS) if it has been determined that DQA1*05:01, DQB1*02:01, and DRB1*03:01 alleles are present.

In some embodiments, the methods include determining a subject's HLA type as described herein, and determining that the subject is predisposed to developing celiac disease (CD) if it has been determined that HLA-DQ2 (encoded by HLA-DQA1*05:01-DQB1*02:01) and/or HLA-DQ8 (encoded by DQA1*03:01-DQB1*03:02) are present.

In an embodiment, the methods include determining a woman's KIR type as described herein, and determining that the woman is predisposed to developing preeclampsia if it has been determined that a KIR2DL1 allele is present.

In an embodiment, both HLA and KIR genotypes may be determined for the same subject. The methods may include determining that the subject is predisposed to clearing an HCV infection, slow progression of HIV infection to AIDS, or Crohn's disease when certain KIR alleles in combination with certain HLA alleles have been found in the individual. In an embodiment, the methods include determining a subject's KIR type as described herein, and determining that the subject is likely to clear an HCV infection if it has been determined that a KIR2DL3 allele is present. In some embodiments, the methods further comprise determining the a subject's HLA-C genotype and determining that the a subject is likely to clear an HCV infection if it has been determined that a combination of KIR2DL3 allele and HLA-C1 allele are present.

In other embodiments, the methods include determining a subject's KIR type as described herein, and determining that the subject is less likely to progress from HIV infection to AIDS if it has been determined that a KIR3DS1 allele is present. In some embodiments, the methods further comprise determining the subject's HLA genotype and determining that the subject is less likely to progress from HIV infection to AIDS if it has been determined that a combination of KIR3DS1 allele and HLA-Bw4 allele are present.

In yet other embodiments, the methods include determining a subject's KIR type as described herein, and determining that the subject is predisposed to developing an autoimmune disease if it has been determined that a KIR2DS1 allele is present.

In yet other embodiments, the methods include determining a subject's KIR type as described herein, and determining that the subject is predisposed to developing Crohn's disease if it has been determined that KIR2DL2/KIR2DL3 heterozygosity is present. In some embodiments, the methods further comprise determining the subject's HLA genotype and determining that the subject is predisposed to developing Crohn's disease if it has been determined that a combination of KIR2DL2/KIR2DL3 heterozygosity and HLA-C2 allele are present.

In yet other embodiments, the methods include determining a subject's KIR type as described herein, and determining whether the subject is a suitable candidate for a donor in an unrelated hematopoietic cell transplantation if it has been determined that a group B KIR haplotype is present.

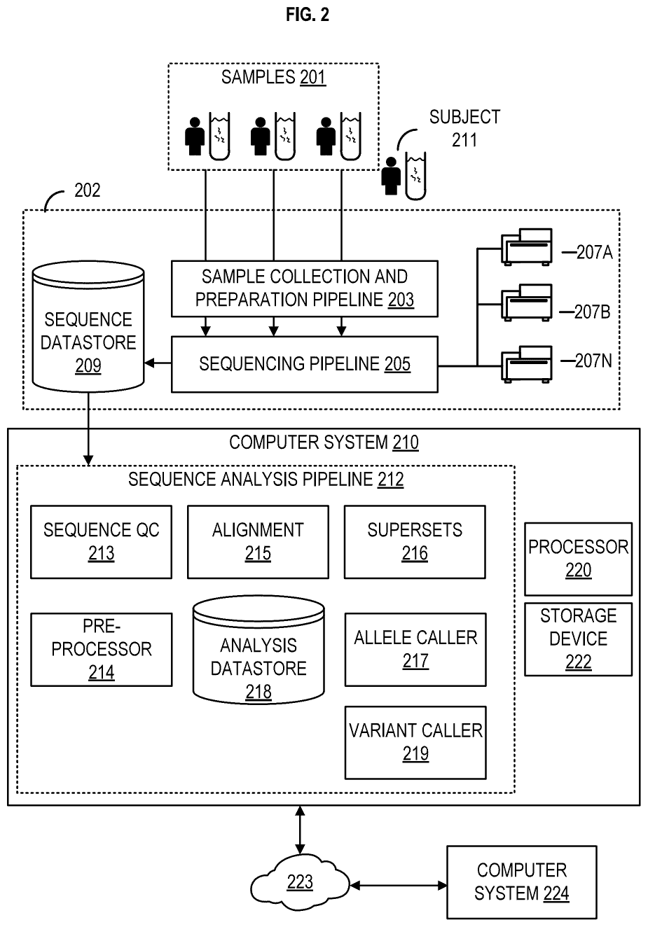

FIG. 2 illustrates an example of a system 200 for determining an allele type and/or a variant of a test subject 211, according to an embodiment of the present disclosure. The system 200 may process one or more samples 201 from the subject 211 to generate sequence reads. The system 200 may include a laboratory system 202, a computer system 210, and/or other components. It should be noted that the laboratory system 202 and the computer system 210 may be remote from one another, and connected to one another through a computer network (not illustrated). The laboratory system 202 may include a sample collection and preparation pipeline 203, a sequencing pipeline 205, a sequence read datastore 209, and/or other components. The sequencing pipeline 205 may include one or more sequencing devices 207 (illustrated in FIG. 2 as sequencing devices 207a . . . n).

The methods of this disclosure may have a wide variety of uses in the manipulation, preparation, identification, quantification, and/or analysis of cell-free nucleic acids. As shown in FIG. 2, the sample collection and preparation pipeline 203 may include obtaining cfDNA reference samples 201 from one or more reference subjects and a cfDNA test sample 211 from a test subject. As described herein, a polynucleotide can comprise any type of nucleic acid, such as DNA and/or RNA. For example, if a polynucleotide is DNA, it can be genomic DNA, complementary DNA (cDNA), or any other deoxyribonucleic acid. A polynucleotide can also be a cell-free nucleic acid such as cell-free DNA (cfDNA). For example, the polynucleotide can be circulating cfDNA. Circulating cfDNA may comprise DNA shed from bodily cells via apoptosis or necrosis. cfDNA shed via apoptosis or necrosis may originate from normal (e.g., healthy) bodily cells.

a. Samples

Isolation and extraction of cell free polynucleotides may be performed through collection of samples using a variety of techniques. A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid sample for analysis is plasma or serum containing cell-free nucleic acids, e.g., cell-free DNA (cfDNA).

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled plasma is typically between about 5 ml to about 20 ml.

The sample can comprise various amounts of nucleic acid. Typically, the amount of nucleic acid in a given sample is equated with multiple genome equivalents. For example, a sample of about 30 ng DNA can contain about 10,000 (104) haploid human genome equivalents and, in the case of cfDNA, about 200 billion (2×1011) individual polynucleotide molecules. Similarly, a sample of about 100 ng of DNA can contain about 30,000 haploid human genome equivalents and, in the case of cfDNA, about 600 billion individual molecules.

In some embodiments, a sample comprises nucleic acids from different sources, e.g., from cells and from cell-free sources (e.g., blood samples, etc.). In some embodiments, a sample comprises nucleic acids from a region of genomic DNA comprising alleles that allows for allele typing, such as HLA alleles or KIR alleles.

Exemplary amounts of cell-free nucleic acids in a sample before amplification typically range from about 1 femtogram (fg) to about 1 microgram (μg), e.g., about 1 picogram (pg) to about 200 nanogram (ng), about 1 ng to about 100 ng, about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning step in which cell-free nucleic acids, as found in solution, are separated from intact cells and other non-soluble components of the bodily fluid. In some of these embodiments, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain embodiments, additional clean up steps are used, such as silica-based columns to remove contaminants or salts. Non-specific bulk carrier nucleic acids, for example, are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps. Additional details regarding cfDNA partitioning and related analysis of epigenetic modifications that are optionally adapted for use in performing the methods disclosed herein are described in, for example, WO 2018/119452, filed Dec. 22, 2017, which is incorporated by reference.

b. Nucleic Acid Tags

In certain embodiments, tags providing molecular identifiers or barcodes are incorporated into or otherwise joined to adapters by chemical synthesis, ligation, or overlap extension PCR, among other methods. In some embodiments, the assignment of unique or non-unique identifiers, or molecular barcodes in reactions follows methods and utilizes systems described in, for example, US patent applications 20010053519, 20030152490, 20110160078, and U.S. Pat. Nos. 6,582,908, 7,537,898, and 9,598,731, which are each incorporated by reference.

Tags are linked (e.g., ligated) to sample nucleic acids randomly or non-randomly. In some embodiments, tags are introduced at an expected ratio of identifiers (e.g., a combination of unique and/or non-unique barcodes) to microwells. For example, the identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some embodiments, the identifiers are loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In certain embodiments, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample. The identifiers are generally unique or non-unique.

One exemplary format uses from about 2 to about 1,000,000 different tags, or from about 5 to about 150 different tags, or from about 20 to about 50 different tags, ligated to both ends of a target nucleic acid molecule. For 20-50×20-50 tags, a total of 400-2500 tags are created. Such numbers of tags are typically sufficient for different molecules having the same start and stop points to have a high probability (e.g., at least 94%, 99.5%, 99.99%, 99.999%) of receiving different combinations of tags.

In some embodiments, identifiers are predetermined, random, or semi-random sequence oligonucleotides. In other embodiments, a plurality of barcodes may be used such that barcodes are not necessarily unique to one another in the plurality. In these embodiments, barcodes are generally attached (e.g., by ligation or PCR amplification) to individual molecules such that the combination of the barcode and the sequence it may be attached to creates a unique sequence that may be individually tracked. As described herein, detection of non-uniquely tagged barcodes in combination with sequence data of beginning (start) and end (stop) portions of sequence reads typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In some embodiments, the nucleic acid molecules (from the sample of polynucleotides) may be tagged with sample indexes and/or molecular barcodes (referred to generally as “tags”). Tags may be incorporated into or otherwise joined to adapters by chemical synthesis, ligation (e.g., blunt-end ligation or sticky-end ligation), or overlap extension polymerase chain reaction (PCR), among other methods. Such adapters may be ultimately joined to the target nucleic acid molecule. In other embodiments, one or more rounds of amplification cycles (e.g., PCR amplification) are generally applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after sequence capturing steps are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps. In some embodiments, the sample indexes are introduced after sequence capturing steps are performed. In some embodiments, molecular barcodes are incorporated to the nucleic acid molecules (e.g. cfDNA molecules) in a sample through adapters via ligation (e.g., blunt-end ligation or sticky-end ligation). In some embodiments, sample indexes are incorporated to the nucleic acid molecules (e.g. cfDNA molecules) in a sample through overlap extension polymerase chain reaction (PCR). Typically, sequence capturing protocols involve introducing a single-stranded nucleic acid molecule complementary to a targeted nucleic acid sequence, e.g., a coding sequence of a genomic region and mutation of such region is associated with a cancer type.

In some embodiments, the tags may be located at one end or at both ends of the sample nucleic acid molecule. In some embodiments, tags are predetermined or random or semi-random sequence oligonucleotides. In some embodiments, the tags may be less than about 500, 200, 100, 50, 20, 10, 9, 8, 7, 6, 5, 4, 3, 2, or 1 nucleotides in length. The tags may be linked to sample nucleic acids randomly or non-randomly.

In some embodiments, each sample is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to create a unique sequence that may be individually tracked. In an embodiment, techniques for discriminating true genomic alterations from technical errors may be used as described in Lee, et al. “Accurate Detection of Rare Mutant Alleles by Target Base-Specific Cleavage with the CRISPR/Cas9 System,” ACS Synth. Biol. 2021, 10, 6, 1451-1464 May 19, 2021, incorporated herein by reference in its entirety. Detection of non-unique molecular barcodes in combination with endogenous sequence information (e.g., the beginning (start) and/or end (stop) genomic location/position corresponding to the sequence of the original nucleic acid molecule in the sample, start and stop genomic positions corresponding to the sequence of the original nucleic acid molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original nucleic acid molecule in the sample) typically allows for the assignment of a unique identity to a particular molecule. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

c. Nucleic Acid Amplification

Sample nucleic acids flanked by adapters are typically amplified by PCR and other amplification methods using nucleic acid primers binding to primer binding sites in adapters flanking a DNA molecule to be amplified as part of the sample collection and preparation pipeline 203. In some embodiments, amplification methods involve cycles of extension, denaturation and annealing resulting from thermocycling, or can be isothermal as, for example, in transcription mediated amplification. Other exemplary amplification methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular tags and/or sample indexes/tags to a nucleic acid molecule using conventional nucleic acid amplification methods. The amplifications are typically conducted in one or more reaction mixtures. Molecular tags and sample indexes/tags are optionally introduced simultaneously, or in any sequential order. In some embodiments, molecular tags and sample indexes/tags are introduced prior to and/or after sequence capturing steps are performed. In some embodiments, only the molecular tags are introduced prior to probe capturing and the sample indexes/tags are introduced after sequence capturing steps are performed. In certain embodiments, both the molecular tags and the sample indexes/tags are introduced prior to performing probe-based capturing steps. In some embodiments, the sample indexes/tags are introduced after sequence capturing steps are performed. Typically, sequence capturing protocols involve introducing a single-stranded nucleic acid molecule complementary to a targeted nucleic acid sequence, e.g., a coding sequence of a genomic region and mutation of such region associated with a cancer type. Typically, the amplification reactions generate a plurality of non-uniquely or uniquely tagged nucleic acid amplicons with molecular tags and sample indexes/tags at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

In some aspects, amplification can occur pre and/or post enrichment.

d. Nucleic Acid Enrichment

In some embodiments, sequences are enriched prior to sequencing the nucleic acids as part of the sample collection and preparation pipeline 203. Enrichment is optionally performed for specific target regions or nonspecifically (“target sequences”). In some embodiments, targeted regions of interest may be enriched with nucleic acid capture probes (“baits”) selected for one or more bait set panels using a differential tiling and capture scheme. In some embodiments, targeted regions of interest may be enriched using CRISPR mediated enrichment. A differential tiling and capture scheme generally uses bait sets of different relative concentrations to differentially tile (e.g., at different “resolutions”) across genomic sections associated with the baits, subject to a set of constraints (e.g., sequencer constraints such as sequencing load, utility of each bait, etc.), and capture the targeted nucleic acids at a desired level for downstream sequencing. These targeted genomic sections of interest optionally include natural or synthetic nucleotide sequences of the nucleic acid construct. In some embodiments, biotin-labeled beads with probes to one or more sections of interest can be used to capture target sequences, and optionally followed by amplification of those sections, to enrich for the regions of interest.

Sequence capture typically involves the use of oligonucleotide probes that hybridize to the target nucleic acid sequence. In certain embodiments, a probe set strategy involves tiling the probes across a section of interest. Such probes can be, for example, from about 60 to about 120 nucleotides in length. The set can have a depth of about 2×, 3×, 4×, 5×, 6×, 8×, 9×, 10×, 15×, 20×, 50× or more. The effectiveness of sequence capture generally depends, in part, on the length of the sequence in the target molecule that is complementary (or nearly complementary) to the sequence of the probe.

In some embodiments, a probe can be designed to be specific to the alleles of interest. Thus, different alleles from the same gene have an equal chance to be captured.

In some aspects, after enrichment, amplification (as described above) can be performed.

e. Nucleic Acid Sequencing

As shown in FIG. 2, after extraction and isolation of cfDNA from samples via the sample collection and preparation pipeline 203, the cfDNA may be sequenced via the sequencing pipeline 205 including one or more sequencing devices 207. Sample nucleic acids, optionally flanked by adapters, with or without prior amplification are generally subject to sequencing. Sequencing methods or commercially available formats that are optionally utilized include, for example, Sanger sequencing, high-throughput sequencing, bisulfite sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLID, Ion Torrent, or nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one more nucleic acid fragment types or sections known to contain alleles of interest. The sequencing reactions can also be performed on any nucleic acid fragment present in the sample. The sequence reactions may provide for sequence coverage of the genome of at least about 5%, 10%, 15%, 20%, 25%, 30%, 40%, 50%, 60%, 70%, 80%, 90%, 95%, 99%, 99.9% or 100% of the genome. In other cases, sequence coverage of the genome may be less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some embodiments, cell-free polynucleotides are sequenced with at least about 1000, 2000, 3000, 4000, 5000, 6000, 7000, 8000, 9000, 10000, 50000, or 100,000 sequencing reactions. In other embodiments, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some embodiments, data analysis is performed on at least about 1000, 2000, 3000, 4000, 5000, 6000, 7000, 8000, 9000, 10000, 50000, or 100,000 sequencing reactions. In other embodiments, data analysis may be performed on less than about 1000, 2000, 3000, 4000, 5000, 6000, 7000, 8000, 9000, 10000, 50000, or 100,000 sequencing reactions. An exemplary read depth is from about 1000 to about 50000 reads per locus (base position).

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U). Exemplary enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including barcodes, and the sequencing determines nucleic acid sequences as well as in-line barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (e.g., sticky end ligation).

The nucleic acid sample is typically contacted with a sufficient number of adapters such that there is a low probability (e.g., <1 or 0.1%) that any two copies of the same nucleic acid receive the same combination of adapter barcodes from the adapters linked at both ends. The use of adapters in this manner permits identification of families of nucleic acid sequences with the same start and stop points on a reference nucleic acid and linked to the same combination of barcodes. Such a family represents sequences of amplification products of a nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent analysis.

Additional details regarding nucleic acid sequencing, including the formats and applications described herein are also provided in, for example, Levy et al., Annual Review of Genomics and Human Genetics, 17: 95-115 (2016), Liu et al., J. of Biomedicine and Biotechnology, Volume 2012, Article ID 251364:1-11 (2012), Voelkerding et al., Clinical Chem., 55: 641-658 (2009), MacLean et al., Nature Rev. Microbiol., 7: 287-296 (2009), Astier et al., J Am Chem Soc., 128(5):1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

In some embodiments, primers used for sequencing can be specific to adaptors added to the ends of the DNA fragments or can be specific to the target region of interest. For example, if the target region of interest is an HLA region then primers to specific HLA genes can be used for sequencing. In some embodiments, the primers can be designed to cover an HLA region comprising multiple HLA alleles. In some embodiments, the primers can be designed to cover an HLA region comprising a single HLA allele.

f. Sequencing Panel

To improve the likelihood of detecting genomic regions of interest the sections of DNA sequenced may comprise a panel of genes or genomic sections that comprise known genomic regions. Selection of a limited section for sequencing (e.g., a limited panel) can reduce the total sequencing needed (e.g., a total amount of nucleotides sequenced). A sequencing panel can target a plurality of different genes or regions, for example, HLA alleles or KIR alleles. Alternatively, DNA may be sequenced by whole genome sequencing (WGS) or other unbiased sequencing method without the use of a sequencing panel. Examples of suitable panel and targets for use in panels can be those comprising allele typing regions such as HLA allele regions and/or KIR allele regions.

In some aspects, a panel that targets a plurality of different genes or genomic regions (e.g., HLA genes, KIR genes, and/or the like) is selected such that a subject can be HLA typed or KIR typed using the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for allele typing in a sample.

Genes included in this panel may comprise one or more of the HLA or KIR genes, such as, but not limited to, HLA-A, HLA-B, HLA-C, HLA-E, HLA-F, HLA-G, HLA-H, HLA-J, HLA-K, HLA-L, HLA-N, HLA-P, HLA-S, HLA-T, HLA-U, HLA-V, HLA-W, HLA-DRA, HLA-DRB1, HLA-DRB2, HLA-DRB3, HLA-DRB4, HLA-DRB5, HLA-DRB6, HLA-DRB7, HLA-DRB8, HLA-DRB9, HLA-DQA1, HLA-DQB1, HLA-DQA2, HLA-DQB2, HLA-DQB3, HLA-DOA, HLA-DOB, HLA-DMA, HLA-DMB, HLA-DPA1, HLA-DPB1, HLA-DPA2, HLA-DPB2, HLA-DPA3, HFE, TAP1, TAP2, PSMB9, PSMB8, MICB, MICA, MICC, MICD, MICE, KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, and KIR3DL3.

Probes for detecting the panel of regions can include those for detecting genomic regions of interest (allele typing regions) as well as nucleosome-aware probes (e.g., KRAS codons 12 and 13) and may be designed to optimize capture based on analysis of cfDNA coverage and fragment size variation impacted by nucleosome binding patterns and GC sequence composition.

Some examples of genomic locations of interest may be chromosomal positions 6p21 or 19q13. In some embodiments, genomic locations used in the methods of the present disclosure comprise at least a portion of at least 5, at least 10, at least 15, at least 20, at least 25, at least 30, at least 35, at least 40, at least 45, or at least 50, of the genes of Table 1.

In some embodiments, the one or more regions in the panel comprise one or more loci from one or a plurality of genes for allele typing. A genomic location may be selected for inclusion in a sequencing panel based on the presence of genes of interest for allele typing, such as HLA typing and KIR typing.

Genes included in the panel for sequencing can include the fully transcribed region, the promoter region, enhancer regions, regulatory elements, and/or downstream sequence. In some embodiments, only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

At least one full exon from each different gene in a panel of genes may be sequenced. In some aspects, all of the exons of a gene may be sequenced. The sequenced panel may comprise all or some exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. In some aspects, a selected panel may comprise all of the exons of a gene. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be 50 kb to 10 Mb in size. The sequencing panel can be 500 kb to 5 Mb in size. The sequencing panel can be at least 10 kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, 150 kb, 200 kb, 250 kb, 300 kb, 350 kb, 400 kb, 450 kb, or 500 kb in size. The sequencing panel may be less than 100 kb, 90 kb, 80 kb, 70 kb, 60 kb, or 50 kb in size. The sequencing panel can be at least 1 Mb, 2 Mb, 3 Mb, 4 Mb, 5 Mb, 6 Mb, 7 Mb, 8 Mb, 9 Mb, or 10 Mb in size.

The panel selected for sequencing can comprise at least 1, 5, 10, 15, 20, 25, 30, 40, 50, 60, 80, or 100 genomic locations (e.g., that each include genomic regions of interest). In some cases, the genomic locations in the panel are selected that the size of the locations are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the genomic locations in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel can comprise one or more locations comprising genomic regions of interest from each of one or more genes. In some cases, the panel can comprise one or more locations comprising genomic regions of interest from each of at least 1, 2, 3, 4, 5, 6, 7, 8, 9, 10, 15, 20, 25, 30, 40, 50, or 80 genes. In some cases, the panel can comprise one or more locations comprising genomic regions of interest from each of at most 1, 2, 3, 4, 5, 6, 7, 8, 9, 10, 15, 20, 25, 30, 40, 50, or 80 genes. In some cases, the panel can comprise one or more locations comprising genomic regions of interest from each of from about 1 to about 80, from 1 to about 50, from about 3 to about 40, from 5 to about 30, from 10 to about 20 different genes.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances this may allow for more molecules within a biological to be analyzed thereby enabling lower frequency alleles to be detected.

In an embodiment, utilizing the sequencing pipeline 205, the panel may be subjected to one or more of: whole-genome bisulfite sequencing (WGBS) interrogating genome-wide methylation patterns, whole-genome sequencing (WGS), and/or targeted sequencing approaches interrogating copy-number variants (CNVs) and single-nucleotide variants (SNVs).

g. Sequence Analysis Pipeline

In an embodiment, after sequencing, sequence reads and any associated data may be stored in the sequence datastore 209. The sequence reads can be stored in any format. The sequence datastore 209 may be local and/or remote to a location where sequencing is performed. As shown in FIG. 2, the stored reads may be subjected to a sequence analysis pipeline 212.

i. Sequence Quality Control

The sequence analysis pipeline 212 may include a sequence quality control (QC) component 213 that may filter sequence reads from the laboratory system 102. The sequence QC component 213 may assign a quality score to one or more sequence reads. A quality score may be a representation of sequence reads that indicates whether those sequence reads may be useful in subsequent analysis based on a threshold. In some cases, some sequence reads are not of sufficient quality or length to perform a subsequent mapping step. Sequence reads with a quality score at least 60%, 70%, 80%, 90%, 95%, 99%, 99.9%, 99.99% or 99.999% may be filtered out of a data set of sequence reads. In other cases, sequence reads assigned a quality scored at least 90%, 95%, 99%, 99.9%, 99.99% or 99.999% may be filtered out of the data set.

Sequence reads that meet a specified quality score threshold may be mapped to a reference genome by the sequence QC component 213. After mapping alignment, sequence reads may be assigned a mapping score. A mapping score may be a representation of sequence reads mapped back to the reference sequence indicating whether each position is or is not uniquely mappable. Sequence reads with a mapping score at least 60%, 70%, 80%, 90%, 95%, 99%, 99.9%, 99.99% or 99.999% may be filtered out of the data set. In other cases, sequencing reads assigned a mapping scored less than 90%, 95%, 99%, 99.9%, 99.99% or 99.999% may be filtered out of the data set.

ii. Pre-Processor

A pre-processor 214 may retrieve/receive data from the analysis datastore 218. For example, the pre-processor 214 may retrieve/receive data representing the plurality of known allele sequences, the plurality of test sequence reads, and/or the plurality of decoy sequences. The pre-processor 214 may also be configured to retrieve sequence data from another source (e.g., an external source). For example, the pre-processor 214 may be configured to download a plurality of known allele sequences, for example from the IPD-IMGT/HLA Database and/or the IPD-KIR Database.

The pre-processor 214 may be configured to divide the known allele sequences into a plurality of k-mer sequences. In other example, k may be from about 25 to about 250. For example, k may be 135 or 140. In an embodiment, k may be 125-175 nucleotides, 130-160 nucleotides, 135-155 nucleotides, 140-150 nucleotides in length. In an embodiment, the k may be 140, 141, 142, 143, 144, or 145 nucleotides in length.

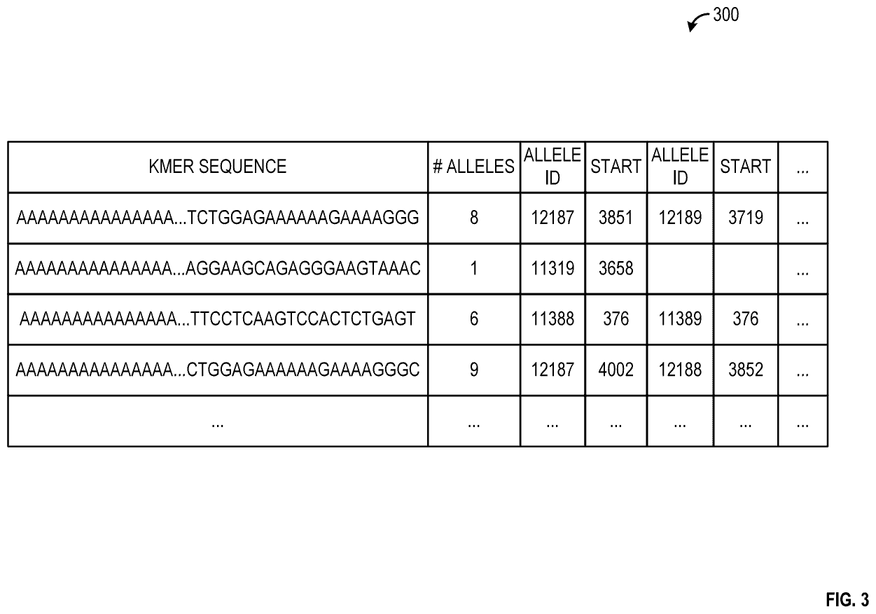

The pre-processor 214 may create a database comprising the k-mer sequences and additional data. The pre-processor 214 may create a data structure comprising the k-mer sequences and additional data. The data structure may be, for example, a table or a flat file. FIG. 3 shows an example data structure 300. The data structure 300 may comprise an entry for each k-mer sequence. For each k-mer sequence, the data structure 300 may indicate the number of alleles the k-mer is associated with and, for each of those alleles, an allele identifier and a starting position where the k-mer sequence is found on that allele. The pre-processor 214 may create a database comprising the decoy sequences and additional data. The pre-processor 214 may create a data structure comprising the decoy sequences and additional data. The data structure may be, for example, a table or a flat file.

iii. Alignment Component

An alignment component 215 may retrieve/receive data from the analysis datastore 218. For example, the alignment component 215 may retrieve/receive data representing the plurality of known allele sequences, k-mer sequences generated from the plurality of known allele sequences, the plurality of test sequence reads, and/or the plurality of decoy sequences.

In various embodiments, the alignment component 215 may be configured to align a test sequence read to a reference sequence or another test sequence read. The alignment component 215 may be configured to align a test sequence read to one or more k-mer sequences generated from the plurality of known allele sequences. The alignment component 215 may be configured to align a test sequence read (e.g., pair) to one or more decoy sequences.

An alignment score is a score indicating a similarity of two sequences determined using an alignment method. In some implementations, an alignment score accounts for number of edits (e.g., deletions, insertions, and substitutions of characters in the string). In some implementations, an alignment score accounts for a number of matches. In some implementations, an alignment score accounts for both the number of matches and a number of edits. In some implementations, the number of matches and edits are equally weighted for the alignment score. For example, an alignment score can be calculated as: # of matches−# of insertions−# of deletions−# of substitutions. In other implementations, the numbers of matches and edits can be weighted differently. For example, an alignment score can be calculated as: # of matches×5−# of insertions×4−# of deletions×4−# of substitutions×6.

Pairwise alignment generally involves placing one sequence along part of target, introducing gaps according to an algorithm, scoring how well the two sequences match, and preferably repeating for various positions along the reference. The best-scoring match is deemed to be the alignment and represents an inference of homology between alignment portions of the sequences. In some embodiments, scoring an alignment of a pair of nucleic acid sequences involves setting values for the scores of substitutions and indels. When individual bases are aligned, a match or mismatch contributes to the alignment score by a substitution probability, which could be, for example, 1 for a match and −0.33 for a mismatch. An indel deducts from an alignment score by a gap penalty, which could be, for example, −1. Gap penalties and substitution probabilities can be based on empirical knowledge or a priori assumptions about how sequences evolve. Their values affect the resulting alignment. Particularly, the relationship between the gap penalties and substitution probabilities influences whether substitutions or indels will be favored in the resulting alignment.

By way of example, the alignment component 215 may utilize a Burrows-Wheeler Aligner (BWA). Generally, the length of the test sequence read can be substantially less than the length of the k-mer sequences generated from the plurality of known allele sequences. The test sequence read and the k-mer sequences can include a sequence of symbols. The alignment of the test sequence read and the k-mer sequences can include a limited number of mismatches between the symbols of the test sequence read and the symbols of the k-mer sequences. Generally, the test sequence read can be aligned to a portion of the k-mer sequences in order to minimize the number of mismatches between the test sequence read and the k-mer sequences.

In particular embodiments, the symbols of the test sequence read and the k-mer sequence can represent the composition of biomolecules. For example, the symbols can correspond to identity of nucleotides in a nucleic acid, such as RNA or DNA. In some embodiments, the symbols can have a direct correlation to these subcomponents of the biomolecules. For example, each symbol can represent a single base of a polynucleotide. In other embodiments, each symbol can represent two or more adjacent subcomponent of the biomolecules, such as two adjacent bases of a polynucleotide. Additionally, the symbols can represent overlapping sets of adjacent subcomponents or distinct sets of adjacent subcomponents. For example, when each symbol represents two adjacent bases of a polynucleotide, two adjacent symbols representing overlapping sets can correspond to three bases of polynucleotide sequence, whereas two adjacent symbols representing distinct sets can represent a sequence of four bases. Further, the symbols can correspond directly to the subcomponents, such as nucleotides, or they can correspond to a color call or other indirect measure of the subcomponents. For example, the symbols can correspond to an incorporation or non-incorporation for a particular nucleotide flow.

In an embodiment, the alignment component 215 may be configured to determine those test sequence reads that have an identical, or substantially identical, alignment to one or more k-mer sequences.

Two nucleic acid sequences or polypeptide sequences are said to be “identical” if the sequence of nucleotides or amino acid residues, respectively, in the two sequences is the same when aligned for maximum correspondence as described herein. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. The term “substantially identical,” used in the context of two nucleic acids or polypeptides, refers to a sequence that has at least 50% sequence identity with a reference sequence. Percent identity can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence using the programs described herein, e.g., BLAST.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.).

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

Nucleic acid or protein sequences that are substantially identical to a reference sequence include “conservatively modified variants.” With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

Once the alignment component 215 has aligned the plurality of test sequence reads to one or more k-mers generated from the known allele sequences, a list of test sequence reads that aligned to (supported) a k-mer sequence of that allele can be generated for each allele. In an embodiment, only test sequence reads that align identically (e.g., no mismatches and no indels) to a k-mer sequence are included in the list. In an embodiment, only test sequence reads that align substantially identically (e.g., at least one mismatch and/or at least one indel) to a k-mer sequence are included in the list. In an embodiment, the alignment component can discard the actual alignment. In an embodiment, a test sequence read may align (identically or substantially identically) to a plurality of alleles. Each test sequence read may be associated with a test sequence read identifier. Accordingly, for each allele, a list of test sequence read identifiers associated with the supporting test sequence reads may be generated. A list of test sequence reads that aligned to a decoy sequence may also be generated. In an embodiment, only test sequence reads that align identically (e.g., no mismatches and no indels) to a decoy sequence are included in the list. In an embodiment, only test sequence reads that align substantially identically (e.g., at least one mismatch and/or at least one indel) to a decoy sequence are included in the list. The alignment component 215 may be configured to discard any test sequence reads that aligned to a decoy sequence with no mismatches and no indels.

iv. Superset Component

A cluster component 216 may retrieve/receive data from the analysis datastore 218. For example, the cluster component 216 may retrieve/receive data representing the plurality of known allele sequences, k-mer sequences generated from the plurality of known allele sequences, the plurality of test sequence reads, and results from the alignment component 215.

In an embodiment, a superset of one or more of the plurality of known allele sequences may be computationally generated by constructing one or more graph data structures. The graph data structure may comprise nodes (also referred to as vertices) representing known allele sequences and edges connecting the nodes indicating that supporting reads of one node are a subset of the supporting reads of the other node. Graph data structure construction may be parallelized given the computationally intensive nature of such construction.

In an embodiment, the graph data structure is stored in a memory subsystem (e.g., FIG. 2, memory 222), which may include pointers to identify a physical location in the memory 222 where each vertex is stored. Typically, the nodes in a graph data structure each represent an element in a set, while the edges represent relationships among the elements. The graph data structure may comprise a directed graph, a tree, a directed acyclic graph (DAG), and/or the like. A directed graph is one in which the edges have a direction. A tree is a type of directed graph data structure having a root node, and a number of additional nodes that are each either an internal node or a leaf node. The root node and internal nodes each have one or more “child” nodes and each is referred to as the “parent” of its child nodes. Leaf nodes do not have any child nodes. Edges in a tree are conventionally directed from parent to child. In a tree, nodes have exactly one parent. A generalization of trees, known as a directed acyclic graph (DAG), allows a node to have multiple parents, but does not allow the edges to form a cycle.

In an embodiment, the graph data structure (superset) may represent a Hasse diagram. For a given locus of the chromosome, the alleles may be sorted by the number of supporting test sequence reads. A graph data structure may be constructed by determining a first allele associated with a highest number of supporting test sequence reads. The first allele may form the basis of the graph data structure (e.g., top level node). The supporting test sequence reads of the first allele may define a set of supporting test sequence reads. Additional alleles may be added to the graph data structure if a given allele is associated with supporting test sequence reads are themselves a subset of the set of supporting test sequence reads of the first allele. Alleles that are not incorporated into the allele superset of the first allele may be used to construct one or more additional allele supersets in a similar fashion. By way of example, a given allele may have the highest number of supporting test sequence reads and each supporting test sequence read may be associated with a test sequence read identifier. In this instance, a set may be formed of the test sequence read identifiers of the supporting test sequence reads for the first allele. In a basic example, the first allele may be supported by test sequence reads having identifiers “1,” “2,” “3,” and “4.” The set of test supporting reads may be expressed as A={1, 2, 3, 4}. The power set of A, P(A), is the set of all subsets of A. For A={1, 2, 3, 4}: P(A)={Ø, {1}, {2}, {3}, {4}, {1, 2}, {1, 3}, {1, 4}, {2, 3}, {2, 4}, {3, 4}, {1, 2, 3}, {1, 2, 4}, {1, 3, 4}, {2, 3, 4}, {1, 2, 3, 4}}.

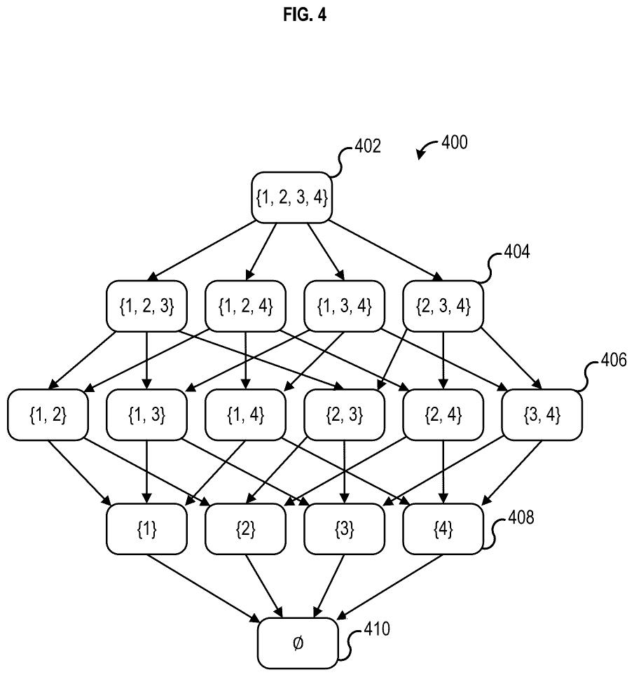

FIG. 4 shows a Hasse diagram 400 of P(A), partially ordered by set inclusion, where X→Y means that Y⊂X. The root node 402 of the Hasse diagram 400 is the set that includes all of test supporting reads 1, 2, 3, and 4. The second-level nodes, such as node 404, are each associated with three-member sets of test supporting reads. The third-level nodes, such as node 406, are each associated with sets containing two test supporting reads. The fourth-level nodes, such as node 408, each contain a single test supporting read. The lowest-level node is the empty set 410.

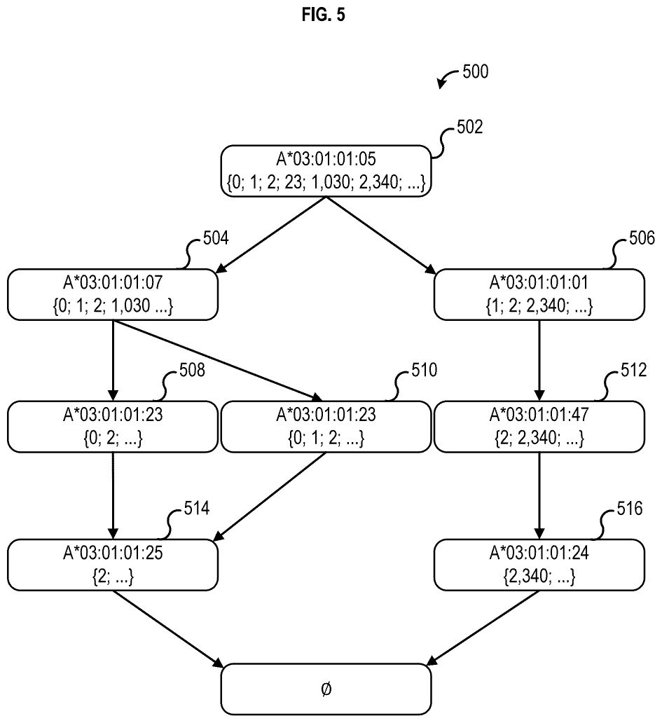

FIG. 5 illustrates construction of an example graph data structure 500 as a Hasse diagram to represent a superset. Allele A*03:01:01:05 represents the known allele sequence with the highest number of supporting test sequence reads. Allele A*03:01:01:05 may represent the first allele inserted as node 502 into the graph data structure 500. Allele A*03:01:01:05 may be associated with supporting test sequence reads having test sequence read identifiers: {0; 1; 2; 23; 1,030; 2,340; . . . }. The remaining known allele sequences may be analyzed to determine which, if any, of the remaining known allele sequences are associated with supporting test sequence reads having test sequence read identifiers that are a subset of {0; 1; 2; 23; 1,030; 2,340; . . . }. In the graph data structure 500, allele A*03:01:01:07 is associated with supporting test sequence reads having test sequence read identifiers {0; 1; 2; 1,030 . . . } which are a subset of {0; 1; 2; 23; 1,030; 2,340; . . . }, resulting in allele A*03:01:01:07 being inserted as node 504 with an edge connecting to node 502 in the graph data structure 500. In the graph data structure 500, allele A*03:01:01:01 is associated with supporting test sequence reads having test sequence read identifiers {1; 2; 2,340; . . . } which are a subset of {0; 1; 2; 23; 1,030; 2,340; . . . }, resulting in allele A*03:01:01:01 being inserted as node 506 with an edge connecting to node 502 in the graph data structure 500. In the graph data structure 500, allele A*03:01:01:23 is associated with supporting test sequence reads having test sequence read identifiers {0; 2; . . . } which are a subset of {0; 1; 2; 1,030 . . . }, resulting in allele A*03:01:01:23 being inserted as node 508 with an edge connecting to node 504 in the graph data structure 500. In the graph data structure 500, allele A*03:01:01:23 is associated with supporting test sequence reads having test sequence read identifiers {0; 1; 2; . . . } which are a subset of {0; 1; 2; 1,030 . . . }, resulting in allele A*03:01:01:23 being inserted as node 510 with an edge connecting to node 504 in the graph data structure 500.

In the graph data structure 500, allele A*03:01:01:47 is associated with supporting test sequence reads having test sequence read identifiers {2; 2,340; . . . } which are a subset of {1; 2; 2,340 . . . }, resulting in allele A*03:01:01:47 being inserted as node 512 with an edge connecting to node 506 in the graph data structure 500. In the graph data structure 500, allele A*03:01:01:25 is associated with supporting test sequence reads having test sequence read identifiers {2; . . . } which are a subset of {0; 2; . . . } and {0; 1; 2; . . . }, resulting in allele A*03:01:01:25 being inserted as node 514 with an edge connecting to node 508 and node 510 in the graph data structure 500. In the graph data structure 500, allele A*03:01:01:24 is associated with supporting test sequence reads having test sequence read identifiers {2,340; . . . } which are a subset of {2; 2,340; . . . } resulting in allele A*03:01:01:24 being inserted as node 516 with an edge connecting to node 512 in the graph data structure 500.

The graph data structure 500 is completed when no further known allele sequences are associated with supporting test sequence reads having test sequence read identifiers that are a subset of node 502. Additional graph data structures may be constructed using the remaining known allele sequences. In an embodiment, once all known allele sequences have been inserted into a graph data structure (superset). The root node (first allele) represents a candidate allele. The candidate allele may be a candidate for the allele present at the locus.

In an embodiment, the graph data structure 500 may be traversed to determine the known allele sequences associated with a given set of test sequence read identifiers.

In an embodiment, the graph data structure (e.g., representing a superset) is stored in a memory subsystem (e.g., FIG. 2, memory 222) using adjacency techniques, which may include pointers to identify a physical location in the memory 222 where each vertex is stored. In an embodiment, the graph data structure is stored in the memory 222 using adjacency lists. In some embodiments, there is an adjacency list for each vertex.

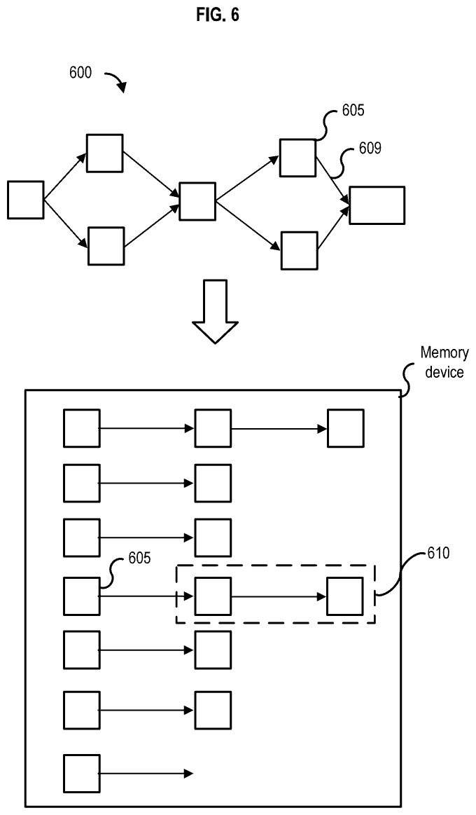

FIG. 6 shows a graph data structure 600 that includes vertex objects 605 and edge objects 609. Known allele sequences and associated identifiers of supporting test sequence reads are identified as blocks and those blocks are transformed into objects 605 that are stored in a tangible memory device. The objects 605 are connected to create paths such that there is a path for each known allele sequence and associated subset of identifiers of supporting test sequence reads. A path may indicate the known allele sequences that are associated with a subset of identifiers of supporting test sequence reads. The connections creating the paths can themselves be implemented as objects so that the blocks are represented by vertex objects 605 and the connections are represented by edge objects 609. Thus the directed graph comprises vertex and edge objects stored in the tangible memory device. As used herein, node object 605 and edge object 609 refer to an object created using a computer system.

FIG. 6 further shows an adjacency list 610 for vertices 605. The disclosed methods and systems may use a processor to create a graph data structure 600 that includes vertex objects 605 and edge objects 609 through the use of adjacency, e.g., adjacency lists or index free adjacency. Thus, the processor may create the graph data structure 600 using index-free adjacency wherein a vertex 605 includes a pointer to another vertex 605 to which it is connected and the pointer identifies a physical location on a memory device 222 where the connected vertex is stored. The graph data structure 600 may be implemented using adjacency lists such that each vertex or edge stores a list of such objects that it is adjacent to. Each adjacency list comprises pointers to specific physical locations within a memory device for the adjacent objects.

The graph data structure 600 will typically be stored on a physical device of memory subsystem 222 in a fashion that provides for very rapid traversals. In that sense, the bottom portion of FIG. 6 represents that objects are stored at specific physical locations on a tangible part of the memory subsystem 222. Each node 605 is stored at a physical location, the location of which is referenced by a pointer in any adjacency list 610 that references that node. Each node 605 has an adjacency list 610 that includes every adjacent node in the graph data structure 600. The entries in the list 610 are pointers to the adjacent nodes.

In certain embodiments, there is an adjacency list for each vertex and edge and the adjacency list for a vertex or edge lists the edges or vertices to which that vertex or edge is adjacent.

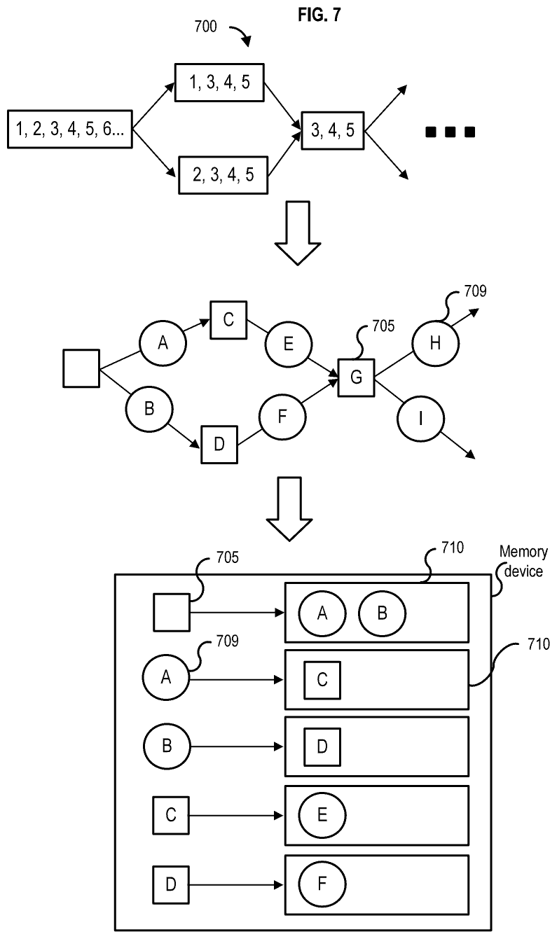

FIG. 7 shows the use of an adjacency list 710 for each vertex 705 and edge 709. As shown in FIG. 7, the disclosed methods and systems may create the graph data structure 700 using an adjacency list 710 for each vertex and edge, wherein the adjacency list 710 for a vertex 705 or edge 709 lists the edges or vertices to which that vertex or edge is adjacent. Each entry in adjacency list 710 is a pointer to the adjacent vertex or edge.

Each pointer identifies a physical location in the memory subsystem at which the adjacent object is stored. In the preferred embodiments, the pointer or native pointer is manipulatable as a memory address in that it points to a physical location on the memory and permits access to the intended data by means of pointer dereference. That is, a pointer is a reference to a datum stored somewhere in memory; to obtain that datum is to dereference the pointer. The feature that separates pointers from other kinds of reference is that a pointer's value is interpreted as a memory address, at a low-level or hardware level. Such a graph representation provides means for fast random access, modification, and data retrieval.

In some embodiments, fast random access is supported and graph object storage are implemented with index-free adjacency in that every element contains a direct pointer to its adjacent elements, which obviates the need for index look-ups, allowing traversals to be very rapid. Index-free adjacency is another example of low-level, or hardware-level, memory referencing for data retrieval. Specifically, index-free adjacency can be implemented such that the pointers contained within elements are references to a physical location in memory.

Since a technological implementation that uses physical memory addressing such as native pointers can access and use data in such a lightweight fashion without the requirement of separate index tables or other intervening lookup steps, the capabilities of a given computer, e.g., any modern consumer-grade desktop computer, are extended to allow for full operation of a genomic-scale graph (e.g., the graph data structure 700 that represents a superset of known allele sequences). Thus storing graph elements (e.g., nodes and edges) using a library of objects with native pointers or other implementation that provides index-free adjacency actually improves the ability of the technology to provide storage, retrieval, and alignment for sequence information since it uses the physical memory of a computer in a particular way.

v. Allele Caller-Selection of Alleles from Supersets

An allele caller 217 may retrieve/receive data from the analysis datastore 218. For example, the allele caller 217 may retrieve/receive data representing the plurality of known allele sequences, k-mer sequences generated from the plurality of known allele sequences, the plurality of test sequence reads, results from the alignment component 215, and/or one or more graph data structures (supersets) generated by the cluster component 216.

The allele caller 217 may be configured to determine an allele type for a given allele. The allele caller 217 may be configured to classify an allele based on the one or more graph data structures (supersets).

In an embodiment, in the event only one graph data structure (superset) is constructed (e.g., all known allele sequences fall within a single graph data structure) the allele (the first allele) associated with the root node of the graph data structure may be classified as the allele present at the locus (e.g., haploid locus) of the chromosome.

In an embodiment, in the event that a plurality of graph data structures (supersets) are constructed, the alleles (the first alleles) associated with the root nodes of the two supersets having a cumulative largest number of distinct supporting test sequence reads may be classified as the alleles present at the locus (e.g., diploid locus) of the chromosome.

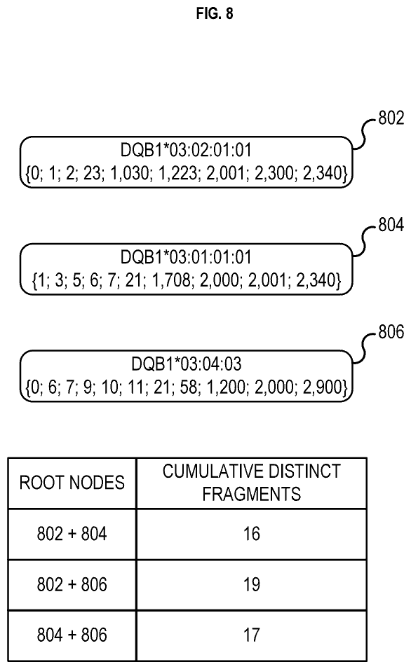

A set operation may be performed on combinations of root nodes to determine the two root nodes having a cumulative largest number of distinct supporting test sequence reads. In an embodiment a union operation (U) may be used. As shown in FIG. 8, cluster component 216 constructed three graph data structures and the resulting root nodes from each graph data structure are shown as root node 802, root node 804 and root node 806. A union operation may be performed between root node 802 and root node 804, between root node 802 and root node 806, and between root node 804 and root node 806. It is contemplated that a root node may be associated with hundreds or thousands of test sequence read identifiers, however, for simplicity of explanation, root node 802 is depicted as being associated with a set of 9 test sequence read identifiers made up of the set {0; 1; 2; 23; 1,030; 1,223; 2,001; 2,300; 2,340}, root node 804 is depicted as being associated with a set of 10 test sequence read identifiers made up of the set {1; 3; 5; 6; 7; 21; 1,708; 2,000; 2,001; 2,340}, and root node 806 is depicted as being associated with a set of 11 test sequence read identifiers made up of the set {0; 6; 7; 9; 10; 11; 21; 58; 1,200; 2,000; 2,900}. A union operation between node 802 and node 804 results in a set {0; 1; 2; 3; 5; 6; 7; 21; 23; 1,030; 1,223; 1,708; 2,000; 2,001; 2,300; 2,340} which includes 16 cumulative distinct test sequence read identifiers, as the union operation discards duplicates. A union operation between node 802 and node 806 results in a set {0; 1; 2; 6; 7; 9; 10; 11; 21; 23; 58; 1,030; 1,200; 1,223; 2,000; 2,001; 2,300; 2,340; 2,900} which includes 19 cumulative distinct test sequence read identifiers, as the union operation discards duplicates. A union operation between node 804 and node 806 results in a set {0; 1; 3; 5; 6; 7; 9; 10; 11; 21; 58; 1,200; 1,708; 2,000; 2,001; 2,340; 2,900} which includes 17 cumulative distinct test sequence read identifiers, as the union operation discards duplicates. The union operation between node 802 and node 806 resulted in the largest cumulative number of distinct test sequence read identifiers, and therefore distinct test sequence reads. Accordingly, the allele caller 217 may classify the known allele sequences associated with node 802 (DQB1*03:02:01:01) and node 806 (DQB1*03:04:03) (e.g., the first alleles) as the alleles present at the locus. The allele caller 217 may continue to process graph data structures/root nodes for any number of loci.

vi. Variant Caller

A variant caller 219 may retrieve/receive data from the analysis datastore 218. For example, the variant caller 219 may retrieve/receive data representing a plurality of sequence reads. The variant caller 219 may retrieve test sequence reads that aligned to a decoy sequence and to a known allele with at least one mismatch and/or at least one indel and that had a greater alignment score to the known allele. The test sequence reads may be analyzed to determine one or more variants. Variants may include, for example, single nucleotide variants (SNVs), indels, fusions, and/or copy number variation. Any known technique for variant calling may be used. In an embodiment, nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, the length of a given cfDNA fragment based upon where its endpoints (i.e., it 5′ and 3′ terminal nucleotides) map to the reference sequence, the offset of a midpoint of a given cfDNA fragment from a midpoint of a genomic region in the cfDNA fragment, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

The variant caller 417 may be configured to assign a somatic or germline label to a given variant. Additional details regarding classification of a variant as somatic or germline that are optionally adapted for use in performing the methods disclosed herein are described in, for example, U.S. Pat. App. Pub. No. US20210265013A1, filed Feb. 17, 2021, which is incorporated by reference. In an embodiment, the germline status may be verified using one or more germline variant callers. Germline variant callers include HAPLOTYPE CALLER, samtools, and/or freebayes. In an embodiment, the somatic status may bverified using one or more somatic variant callers. Somatic variant callers include SEURAT, STRELKA, and/or MUTECT.

Any data analyzed, determined, and/or output by the sequence analysis pipeline 212 may be stored in the analysis datastore 218. Generally speaking, the processor 220 may implement (be programmed by) various components of the sequence analysis pipeline 212, such as the sequence quality control component 213, the pre-processor 214, the alignment component 215, the cluster component 216, the allele caller 217, the variant caller 219, and/or other components. Alternatively, it should be noted that these components of the sequence analysis pipeline 212 may include a hardware module. Although illustrated separately for convenience, one or more of the various components or instructions, such as the sequence quality control component 213, the pre-processor 214, the alignment component 215, the cluster component 216, the allele caller 217, and/or the variant caller 219 may be integrated with one another.

The computer system 210 may exchange data with a computer system 224 using a network 223. For example, the computer system 224 may retrieve data from the analytics datastore 218. The computer system 224 may be configured for determining/classifying alleles present at a locus.

h. Example Methods

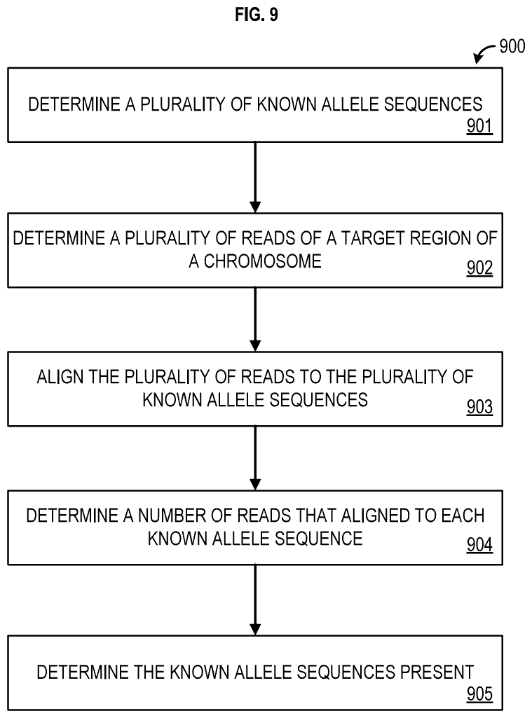

In an embodiment, shown in FIG. 9, a method 900 for allele calling is disclosed. In an embodiment, the sequence QC component 213, the pre-processor 214, the alignment component 215, the cluster component 216, the allele caller 217, the variant caller 219, additional components not shown (e.g., a component of the computer system 210) alone and/or in a combination thereof may be configured to access the sequence datastore 209 and/or the analysis datastore 218 and perform the method 900 in whole and/or in part. The method 900 may be performed in whole or in part by a single computing device using parallel computing, in one or more cores, or using a plurality of computing devices, for example using a distributed computer system, and the like.

The method 900 may comprise determining a plurality of known allele sequences at 901. The plurality of known allele sequences may comprise a plurality of known human leukocyte antigen (HLA) allele sequences or a plurality of known killer cell immunoglobulin-like receptor (KIR) region allele sequences. Determining the plurality of known allele sequences may comprise retrieving sequence data for known alleles, determining, based on the sequence data for known alleles, a plurality of k-mer sequences, and assembling the plurality of k-mer sequences into a data structure comprising a plurality of entries, wherein each entry may comprise a k-mer sequence, a number of alleles containing the k-mer sequence, an allele identifier for each allele containing the k-mer sequence, and a start and stop position of the k-mer sequence in each allele.

The method 900 may comprise determining a plurality of sequence reads of a target region of a chromosome of a subject at 902. The target region of the chromosome may comprise one or more loci. The method 900 may further comprise obtaining a sample from the subject and sequencing the sample to obtain the plurality of sequence reads of the target region of the chromosome. The target region may comprise one or more of the following genes: HLA-A, HLA-B, HLA-C, HLA-E, HLA-F, HLA-G, HLA-H, HLA-J, HLA-K, HLA-L, HLA-N, HLA-P, HLA-S, HLA-T, HLA-U, HLA-V, HLA-W, HLA-DRA, HLA-DRB1, HLA-DRB2, HLA-DRB3, HLA-DRB4, HLA-DRB5, HLA-DRB6, HLA-DRB7, HLA-DRB8, HLA-DRB9, HLA-DQA1, HLA-DQB1, HLA-DQA2, HLA-DQB2, HLA-DQB3, HLA-DOA, HLA-DOB, HLA-DMA, HLA-DMB, HLA-DPA1, HLA-DPB1, HLA-DPA2, HLA-DPB2, HLA-DPA3, HFE, TAP1, TAP2, PSMB9, PSMB8, MICB, MICA, MICC, MICD, MICE, KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, or KIR3DL3. The target region may comprise chromosomal position 6p21 or chromosomal position 19q13. The target region may comprise at least a portion of a major histocompatibility complex (MHC) region and the chromosome is human chromosome 6. The target region may comprise at least a portion of a killer cell immunoglobulin-like receptor (KIR) region and the chromosome is human chromosome 19.

The method 900 may comprise aligning the plurality of sequence reads to the plurality of known allele sequences at 903.

The method 900 may comprise determining, based on the alignment, for each known allele sequence of the plurality of known allele sequences, a number of sequence reads that aligned to each known allele sequence at 904. In some embodiments, the sequence reads that aligned to each known allele sequence can be grouped into one or more sequence read families (i.e., based on molecular barcode and genomic location). Accordingly, a number of sequence read families that aligned to each known allele sequence may be determined.

Determining, based on the alignment, for each known allele sequence of the plurality of known allele sequences, a number of sequence reads that aligned to each known allele sequence may comprise determining the number of sequence reads that aligned to each known allele sequence with 100% identity. The method 900 may comprise determining a number of sequence read families that aligned to each known allele sequence at 904. Determining the number of sequence read families that aligned to each known allele sequence may comprise grouping reads based on barcode.

The method 900 may comprise determining, based on the numbers of sequence reads that aligned to each known allele sequence, for the one or more loci, the known allele sequences present at the one or more loci at 905. The method 900 may comprise determining, based on the numbers of sequence read families that aligned to each known allele sequence, for the one or more loci, the known allele sequences present at the one or more loci at 905. The known allele sequences present at the one or more loci may comprise a human leukocyte antigen (HLA) type at the locus or a killer cell immunoglobulin-like receptor (KIR) type at the locus.

Determining, based on the numbers of sequence reads that aligned to each known allele sequence, for the one or more loci, the known allele sequences present at the one or more loci may comprise determining one or more known allele sequences having a highest number of sequence reads aligned. Determining, based on the numbers of sequence read families that aligned to each known allele sequence, for the one or more loci, the known allele sequences present at the one or more loci may comprise determining one or more known allele sequences having a highest number of sequence read families aligned.

The method 900 may further comprise determining, based on the alignment, for each read of the plurality of sequence reads, one or more known allele sequences to which each read aligns.

The method 900 may further comprise determining a length of a portion of each known allele sequence aligned to two or more sequence reads of the plurality of sequence reads.

The method 900 may further comprise sorting, for a locus, the known allele sequences present at the locus by the number of sequence reads that aligned to each known allele sequence, determining, for the locus, a first known allele sequence with a highest number of sequence reads aligned, inserting the first known allele sequence with the highest number of sequence reads aligned into a superset, determining one or more known allele sequences that aligned to reads that are a subset of the reads aligned to the first known allele sequence, and inserting the one or more known allele sequences into the superset. The superset may comprise a graph data structure. The graph data structure may comprise a directed acyclic graph. The graph data structure may represent a Hasse diagram.

The method 900 may further comprise determining that the locus is associated with a single superset and determining the first known allele sequence of the single superset as the allele present at the locus.

The method 900 may further comprise determining a plurality of supersets for the locus. The method 900 may further comprise determining, based on the plurality of supersets for the locus, two supersets with a cumulative largest number of distinct reads and determining the first known allele sequence of each of the two supersets as the alleles present at the locus.

The method 900 may further comprise, assisting in a communication of the known allele sequences present at the one or more loci to a medical provider.

The method 900 may further comprise obtaining a sample from a cell to be transplanted into the subject and sequencing the sample to obtain the plurality of sequence reads of the target region of the chromosome. The method 900 may further comprise determining if an HLA type of the subject matches the HLA type at the locus. The method 900 may further comprise transplanting the cell into the subject if the HLA type of the subject matches the HLA type at the locus.

The method 900 may further comprise transplanting the cell into the subject if the HLA type of the subject matches the HLA type at the locus and administering an agent that reduces a likelihood of transplant rejection in the subject.

The method 900 may further comprise determining that the subject is predisposed to developing rheumatoid arthritis (RA) wherein the known allele sequences present at the one or more loci may comprise HLA-DRB1*04:01, *04:04, and *04:08; HLA-DRB1*04:05; HLA-DRB1*01:01 and *01:02; HLA-DRB1*14:02; HLA-DRB1*10:01; and HLA-DRB1*01:01, *04:01, *04:04, and *04:05.

The method 900 may further comprise determining that the subject is predisposed to developing multiple sclerosis (MS) wherein the known allele sequences present at the one or more loci may comprise HLA-DRB1*15:01, HLA-DQB1*06:02, HLA-DRB1*01:08, HLA-DRB1*03:01, and/or HLA-DRB1*13:03.

The method 900 may further comprise determining that the subject is predisposed to developing systemic lupus erythematosus (SLE) wherein the known allele sequences present at the one or more loci may comprise HLA-DRB1, HLA-DR2 (DRB1*15:01), HLA-DR3 (DRB1*03:01), HLA-DRB1*08:01, and/or HLA-DQA1*01:02.

The method 900 may further comprise determining that the subject is predisposed to developing Type 1 diabetes mellitus (TID) wherein the known allele sequences present at the one or more loci may comprise rise HLA-DQB1*03:02 and/or DQB1*02:01.

The method 900 may further comprise determining that the subject is predisposed to developing Sjögren's syndrome (SS) wherein the known allele sequences present at the one or more loci may comprise DQA1*05:01, DQB1*02:01, and DRB1*03:01.

The method 900 may further comprise determining that the subject is predisposed to developing that the subject is predisposed to developing celiac disease (CD) wherein the known allele sequences present at the one or more loci may comprise HLA-DQ2 (encoded by HLA-DQA1*05:01-DQB1*02:01) and/or HLA-DQ8 (encoded by DQA1*03:01-DQB1*03:02).

The method 900 may further comprise determining that the subject is predisposed to developing preeclampsia wherein the known allele sequences present at the one or more loci may comprise KIR2DL1.

The method 900 may further comprise determining that the subject is less likely to progress from HIV infection to AIDS wherein the known allele sequences present at the one or more loci may comprise KIR3DS1.

The method 900 may further comprise determining that the subject is less likely to progress from HIV infection to AIDS wherein the known allele sequences present at the one or more loci may comprise a combination of KIR3DS1 and HLA-Bw4.

The method 900 may further comprise determining that the subject is predisposed to developing an autoimmune disease wherein the known allele sequences present at the one or more loci may comprise KIR2DS1.

The method 900 may further comprise determining that the subject is predisposed to developing Crohn's disease wherein the known allele sequences present at the one or more loci may comprise KIR2DL2/KIR2DL3 heterozygosity.

The method 900 may further comprise determining that the subject is predisposed to developing Crohn's disease wherein the known allele sequences present at the one or more loci may comprise a combination of KIR2DL2/KIR2DL3 heterozygosity and HLA-C2 allele.

The method 900 may further comprise determining whether the subject is a suitable candidate for a donor in an unrelated hematopoietic cell transplantation wherein the known allele sequences present at the one or more loci may comprise a group B KIR haplotype.

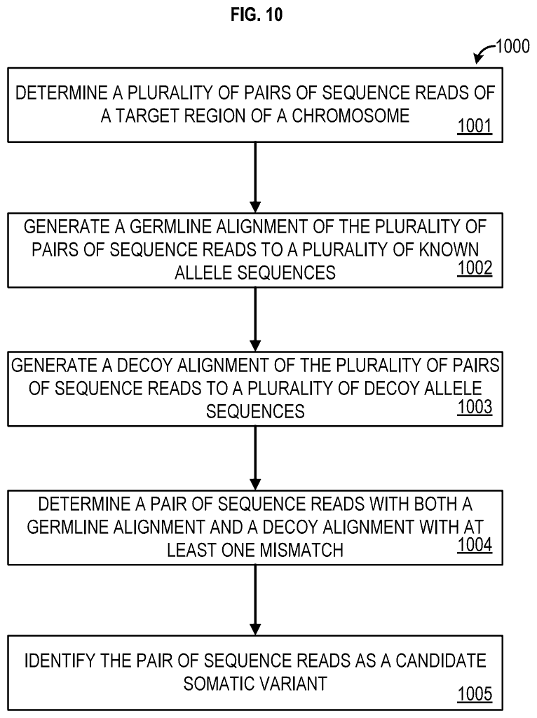

In an embodiment, shown in FIG. 10, a method 1000 for allele and/or variant calling is disclosed. In an embodiment, the sequence QC component 213, the pre-processor 214, the alignment component 215, the cluster component 216, the allele caller 217, the variant caller 219, additional components not shown (e.g., a component of the computer system 210) alone and/or in a combination thereof may be configured to access the sequence datastore 209 and/or the analysis datastore 218 and perform the method 900 in whole and/or in part. The method 900 may be performed in whole or in part by a single computing device using parallel computing, in one or more cores, or using a plurality of computing devices, for example using a distributed computer system, and the like.

The method 1000 may comprise determining a plurality of pairs of sequence reads of a target region of a chromosome of a subject, wherein the target region of the chromosome comprises one or more loci at 1001. The method 1000 may further comprise obtaining a sample from the subject and sequencing the sample to obtain the plurality of pairs of sequence reads of the target region of the chromosome. The target region may comprise one or more of the following genes: HLA-A, HLA-B, HLA-C, HLA-E, HLA-F, HLA-G, HLA-H, HLA-J, HLA-K, HLA-L, HLA-N, HLA-P, HLA-S, HLA-T, HLA-U, HLA-V, HLA-W, HLA-DRA, HLA-DRB1, HLA-DRB2, HLA-DRB3, HLA-DRB4, HLA-DRB5, HLA-DRB6, HLA-DRB7, HLA-DRB8, HLA-DRB9, HLA-DQA1, HLA-DQB1, HLA-DQA2, HLA-DQB2, HLA-DQB3, HLA-DOA, HLA-DOB, HLA-DMA, HLA-DMB, HLA-DPA1, HLA-DPB1, HLA-DPA2, HLA-DPB2, HLA-DPA3, HFE, TAP1, TAP2, PSMB9, PSMB8, MICB, MICA, MICC, MICD, MICE, KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, or KIR3DL3. The target region may comprise chromosomal position 6p21 or chromosomal position 19q13. The target region may comprise at least a portion of a major histocompatibility complex (MHC) region and the chromosome is human chromosome 6. The target region may comprise at least a portion of a killer cell immunoglobulin-like receptor (KIR) region and the chromosome is human chromosome 19.

The method 1000 may comprise generating a germline alignment of the plurality of pairs of sequence reads to a plurality of known allele sequences at 1002. The plurality of known allele sequences may comprise a plurality of known human leukocyte antigen (HLA) allele sequences or a plurality of known human killer cell immunoglobulin-like receptor (KIR) region allele sequences.

The method 1000 may comprise generating a decoy alignment of the plurality of pairs of sequence reads to a plurality of decoy allele sequences at 1003. The plurality of decoy sequences may comprise a plurality of non-human sequences. The plurality of non-human sequences may comprise one or more of a plurality of bovine sequences, a plurality of rat sequences, or a plurality of microbial sequences.

Generating the germline alignment of the plurality of pairs of sequence reads to a plurality of known allele sequences may comprise determining, based on the germline alignment, for a pair of sequence reads of the plurality of pairs of sequence reads, one or more known allele sequences to which each read of the pair of sequence reads aligns with no mismatch or indel.

The method 1000 may further comprise determining, based on the germline alignment, for each known allele sequence of the plurality of known allele sequences, a number of pairs of sequence reads of the plurality of pairs of sequence reads that aligned to each known allele sequence and determining, based on the numbers of pairs of sequence reads that aligned to each known allele sequence, for the one or more loci, the known allele sequences present at the one or more loci.

Generating the decoy alignment of the plurality of pairs of sequence reads to a plurality of decoy allele sequences may comprise determining, based on the decoy alignment, for the pair of sequence reads of the plurality of pairs of sequence reads, one or more decoy allele sequences to which each read of the pair of sequence reads aligns with no mismatch or indel and discarding the pair of sequence reads.

Generating the decoy alignment of the plurality of pairs of sequence reads to a plurality of decoy allele sequences may comprise determining, based on the decoy alignment, for the pair of sequence reads of the plurality of pairs of sequence reads, one or more non-human decoy sequences to which each read of the pair of sequence reads aligns with no mismatch or indel and identifying the plurality of pairs of sequence reads as originating from a contaminated sample.

Generating the germline alignment of the plurality of pairs of sequence reads to a plurality of known allele sequences may comprise determining, based on the germline alignment, for a pair of sequence reads of the plurality of pairs of sequence reads, one or more known allele sequences to which each read of the pair of sequence reads aligns with at least one mismatch or indel and generating the germline alignment score.

Generating the decoy alignment of the plurality of pairs of sequence reads to a plurality of decoy allele sequences may comprise determining, based on the decoy alignment, for the pair of sequence reads of the plurality of pairs of sequence reads, one or more decoy allele sequences to which each read of the pair of sequence reads aligns with at least one mismatch or indel and generating the decoy alignment score.

Generating a germline alignment of the plurality of pairs of sequence reads to a plurality of known allele sequences may comprise determining a pair of sequence reads aligns to at least two allele sequences of the plurality of known allele sequences and selecting one known allele sequence of the at least two allele sequences.

Generating a decoy alignment of the plurality of pairs of sequence reads to a plurality of decoy allele sequences may comprise determining a pair of sequence reads align to at least two decoy allele sequences of the plurality of decoy allele sequences and selecting one decoy allele sequence of the at least two decoy allele sequences.

The method 1000 may comprise determining a pair of sequence reads of the plurality of pairs of sequence reads with both a germline alignment with at least one mismatch and/or indel and a decoy alignment with at least one mismatch and/or indel, wherein the pair of sequence reads is associated with a germline alignment score greater than a decoy alignment score at 1004.

The method 1000 may comprise identifying the pair of sequence reads as a candidate somatic variant at 1005. Identifying the pair of sequence reads as a candidate somatic variant may comprise identifying, based on aligning the pair of sequence reads to one or more reference variants, a reference variant of the one or more reference variants as the candidate variant. Identifying the pair of sequence reads as a candidate somatic variant may comprise identifying, based on aligning the pair of sequence reads to one or more non-human reference variants, a non-human reference variant of the one or more non-human reference variants as the candidate variant and identifying the plurality of pairs of sequence reads as originating from a contaminated sample.

### IV. SYSTEMS AND COMPUTER READABLE MEDIA

The various processing operations and/or methods depicted in the Figures may be accomplished using some or all of the system components described in detail herein and, in some implementations, various operations may be performed in different sequences and various operations may be omitted. Additional operations may be performed along with some or all of the operations shown in the depicted flow diagrams. One or more operations may be performed simultaneously. Accordingly, the operations as illustrated (and described in greater detail herein) are provided as example and, as such, should not be viewed as limiting.

The present methods can be computer-implemented, such that any or all of the operations described in the specification or appended claims other than wet chemistry steps can be performed in a suitable programmed computer. The computer can be a mainframe, personal computer, tablet, smart phone, cloud, online data storage, remote data storage, or the like. The computer can be operated in one or more locations.

Various operations of the present methods can utilize information and/or programs and generate results that are stored on computer-readable media (e.g., hard drive, auxiliary memory, external memory, server; database, portable memory device (e.g., CD-R, DVD, ZIP disk, flash memory cards), and the like.

The present disclosure also includes an article of manufacture for analyzing a nucleic acid population that includes a machine-readable medium containing one or more programs which when executed implement the steps of the present methods.

The disclosure can be implemented in hardware and/or software. For example, different aspects of the disclosure can be implemented in either client-side logic or server-side logic. The disclosure or components thereof can be embodied in a fixed media program component containing logic instructions and/or data that when loaded into an appropriately configured computing device cause that device to perform according to the disclosure. A fixed media containing logic instructions can be delivered to a viewer on a fixed media for physically loading into a viewer's computer or a fixed media containing logic instructions may reside on a remote server that a viewer accesses through a communication medium to download a program component.

The present disclosure provides computer control systems that are programmed to implement methods of the disclosure. Returning to FIG. 2, the processor 220 may include a single core or multi core processor, or a plurality of processors for parallel processing. The storage device 222 may include random-access memory, read-only memory, flash memory, a hard disk, and/or other type of storage. The computer system 210 may include a communication interface (e.g., network adapter) for communicating with one or more other systems, and peripheral devices, such as cache, other memory, data storage and/or electronic display adapters. The components of the computer system 210 may communicate with one another through an internal communication bus, such as a motherboard. The storage device 222 may be a data storage unit (or data repository) for storing data. The computer system 210 may be operatively coupled to a network 223 (“network”) with the aid of the communication interface. The network 223 may be the Internet, an internet and/or extranet, or an intranet and/or extranet that is in communication with the Internet. The network 223 in some cases is a telecommunication and/or data network. The network 223 may include a local area network. The network 23 may include one or more computer servers, which can enable distributed computing, such as cloud computing. The network 223, in some cases with the aid of the computer system 210, may implement a peer-to-peer network, which may enable devices coupled to the computer system 220 to behave as a client or a server. The computer system 210 may exchange data with a computer system 224 using the network 223. For example, the computer system 224 may retrieve data from the analytics datastore 218.

The processor 220 may execute a sequence of machine-readable instructions, which can be embodied in a program or software. The instructions may be stored in a memory location, such as the storage device 222. The instructions can be directed to the processor 220, which can subsequently program or otherwise configure the processor 220 to implement methods of the present disclosure. Examples of operations performed by the processor 220 may include fetch, decode, execute, and writeback.

The processor 220 may be part of a circuit, such as an integrated circuit. One or more other components of the system 200 may be included in the circuit. In some cases, the circuit may include an application specific integrated circuit (ASIC).

The storage device 222 may store files, such as drivers, libraries, and saved programs. The storage device 222 can store user data, e.g., user preferences and user programs. The computer system 210 in some cases may include one or more additional data storage units that are external to the computer system 210, such as located on a remote server that is in communication with the computer system 210 through an intranet or the Internet.

The computer system 210 can communicate with one or more remote computer systems through the network. For instance, the computer system 210 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 210 via the network.

Methods as described herein can be implemented by way of machine (e.g., computer processor) executable code stored on an electronic storage location of the computer system 210, such as, for example, on the storage device 222. The machine executable or machine readable code can be provided in the form of software (e.g., computer readable media). During use, the code can be executed by the processor 220. In some cases, the code can be retrieved from the storage device 222 and stored on the storage device 222 for ready access by the processor 220.

The code may be pre-compiled and configured for use with a machine having a processer adapted to execute the code, or can be compiled during runtime. The code can be supplied in a programming language that can be selected to enable the code to execute in a precompiled or as-compiled fashion.

Aspects of the systems and methods provided herein, such as the computer system 210, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible, storage media, “media” may include other types of (intangible) media.

“Storage” media, terms such as computer or machine “readable medium” refer to any tangible (such as physical), non-transitory, medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 210 can include or be in communication with an electronic display 935 that comprises a user interface (UI) for providing, for example, a report. Examples of UI's include, without limitation, a graphical user interface (GUI) and web-based user interface.

Methods and systems of the present disclosure can be implemented by way of one or more algorithms. An algorithm can be implemented by way of software upon execution by the processor 220.

### V. EXAMPLES

### Example 1

Disclosed herein is an example of accurately genotyping HLA and KIR alleles using cfDNA assay and k-mer based algorithm for immunotherapy.

HLA and KIR genotypes show great promise as emerging biomarkers for immune checkpoint inhibitors (ICIs) and understanding patient prognosis. Multiple studies have shown that HLA-I heterozygosity and high sequence divergence across alleles positively correlates with response to ICIs. However the high degree of polymorphism and allele sequence similarities in HLA and KIR present a challenge to accurate allele calling. To address these difficulties, Kmerizer, a novel allele caller optimized for short fragments, such as germline reads from a cfDNA assay, was developed.

### Methods and Results

HLA and KIR typing using Kmerizer can include an HLA and KIR panel coverage comprising HLA locus: A, B, C, DPA1, DPB1, DQA1, DQB1, DRB1, DRB3, DRB4, DRB5, E, F, G; and KIR locus: 2DL1, 2DL2, 2DL3, 2DL4, 2DL5, 2DP1, 2DS1, 2DS2, 2DS3, 2DS4, 2DS5, 3DL1, 3DL2, 3DL3, 3DP1, 3DS1.

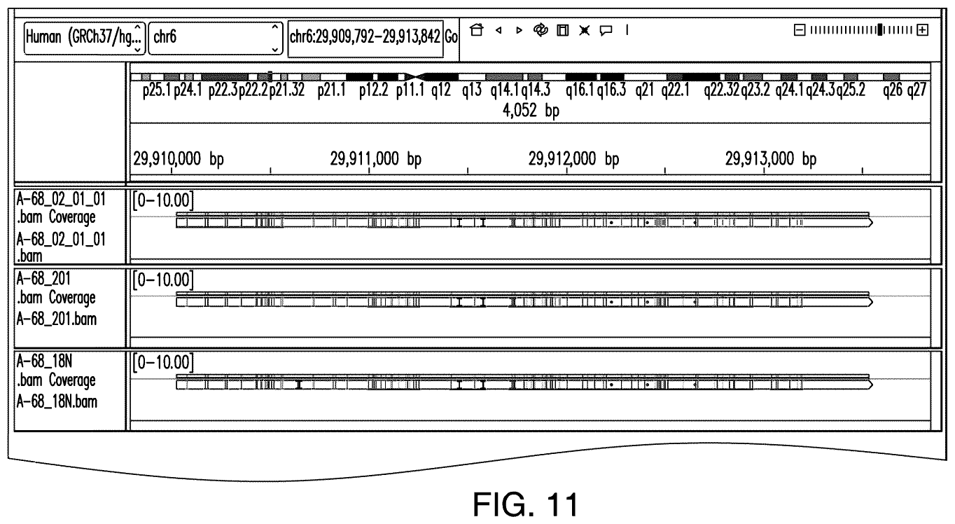

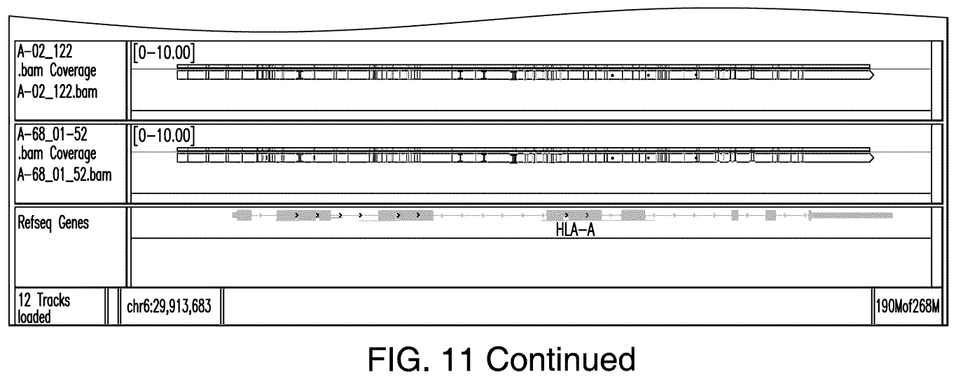

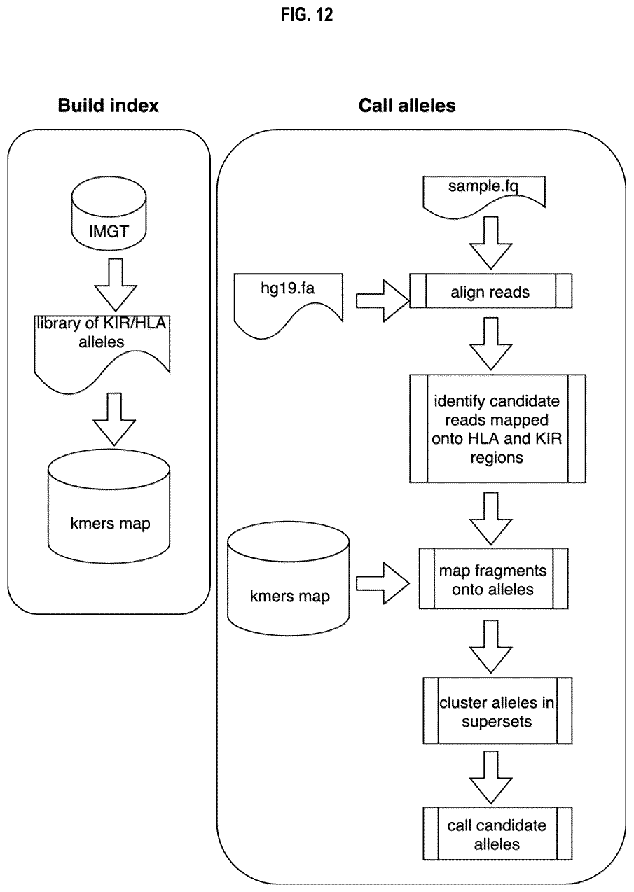

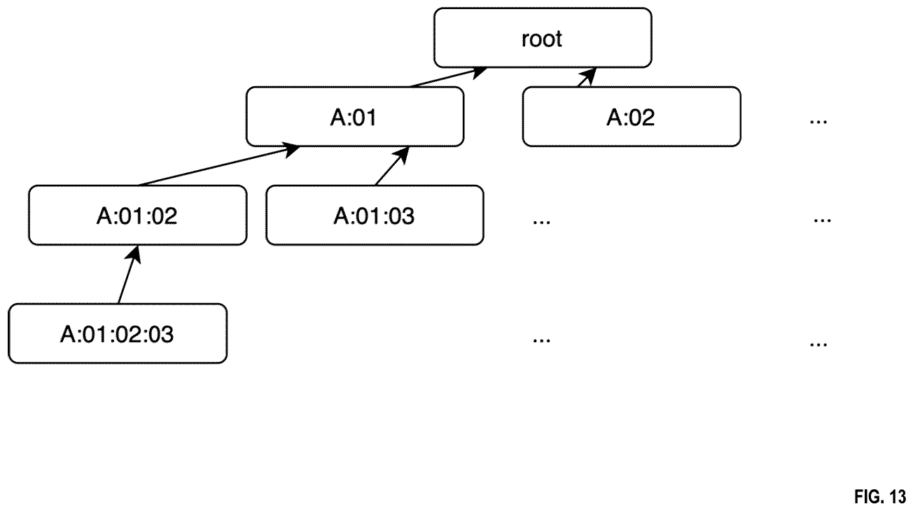

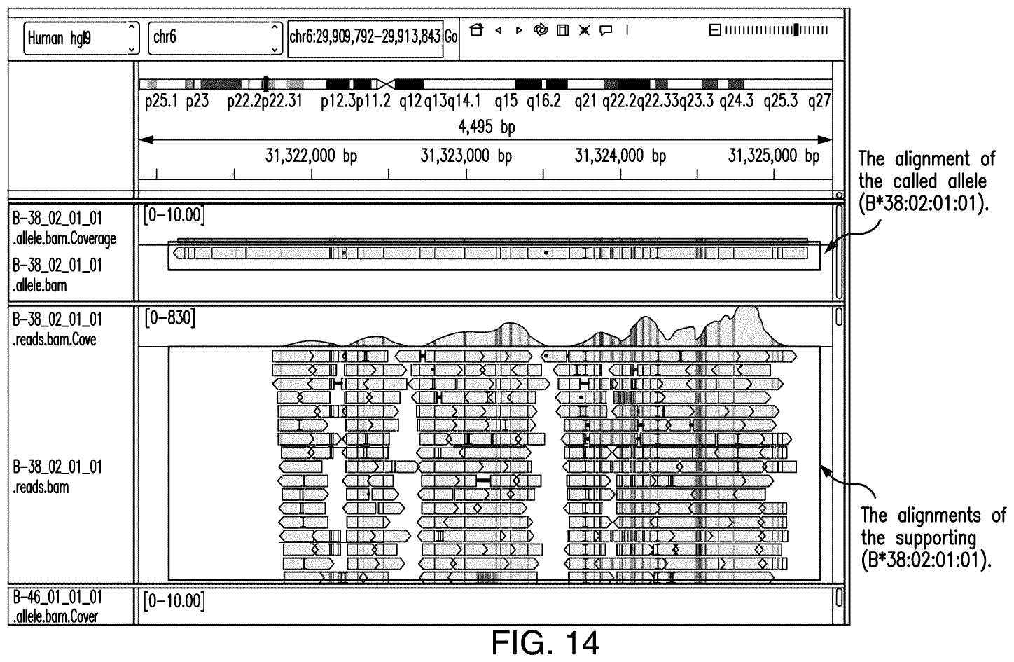

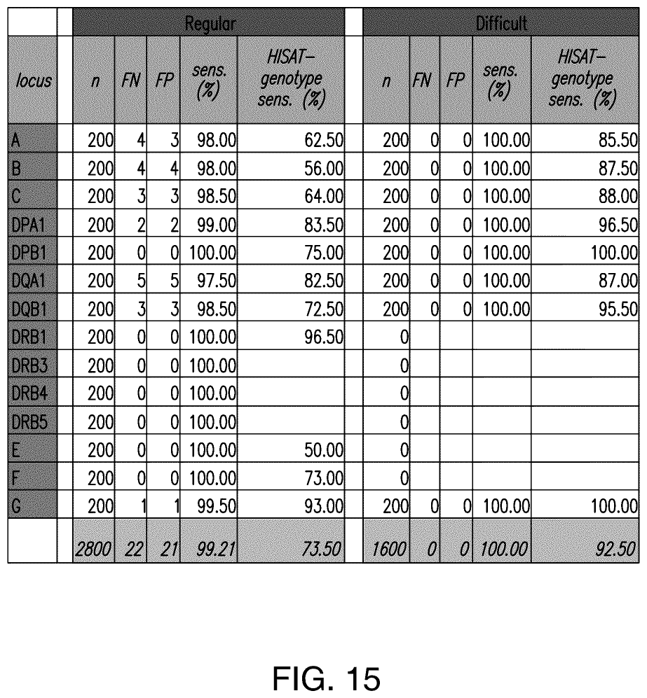

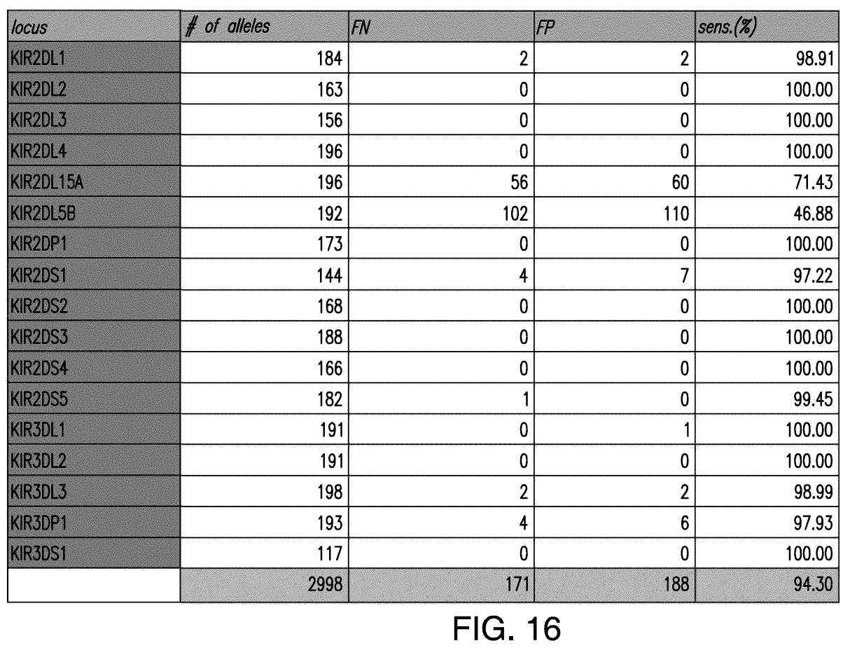

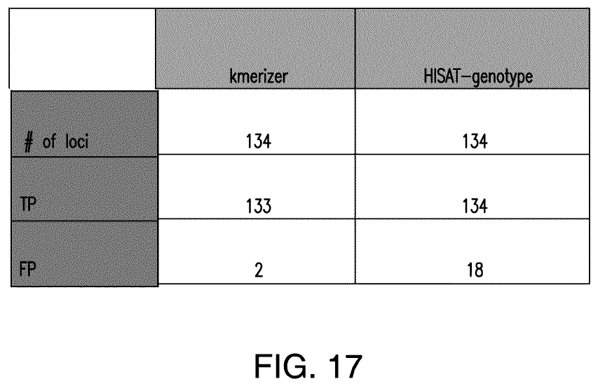

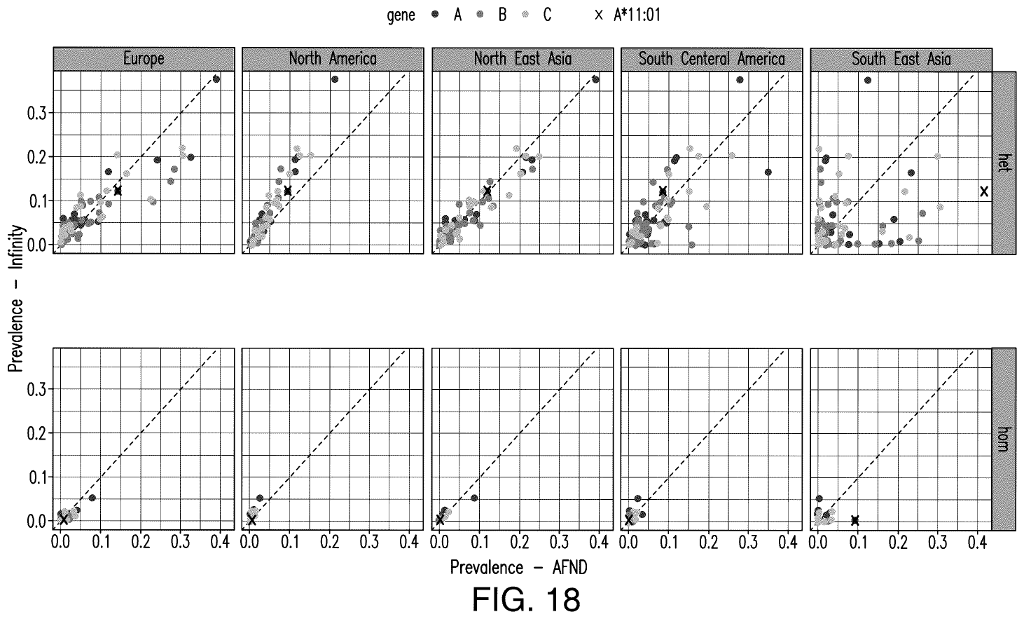

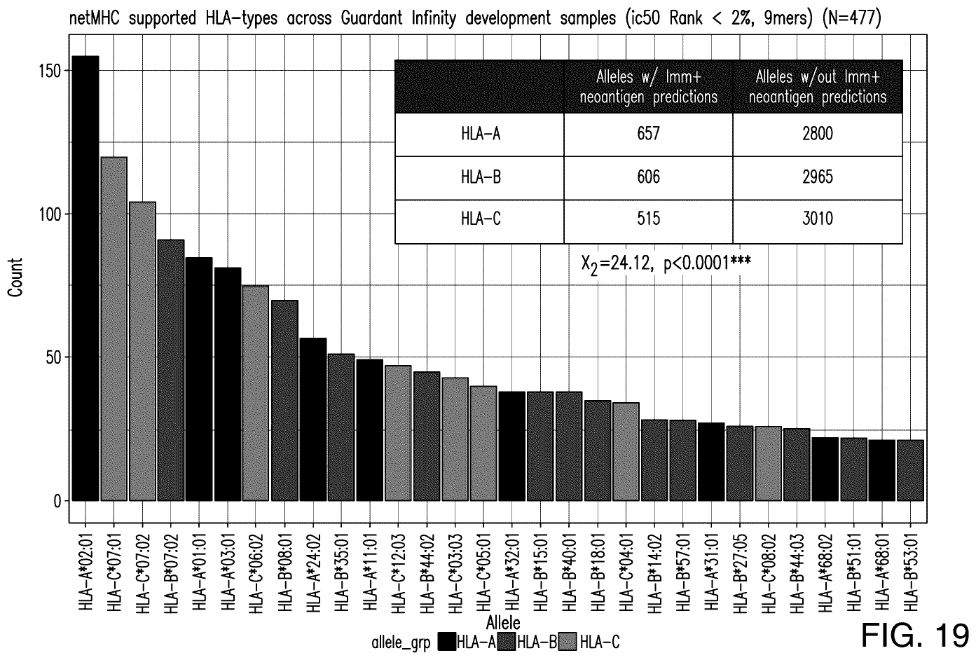

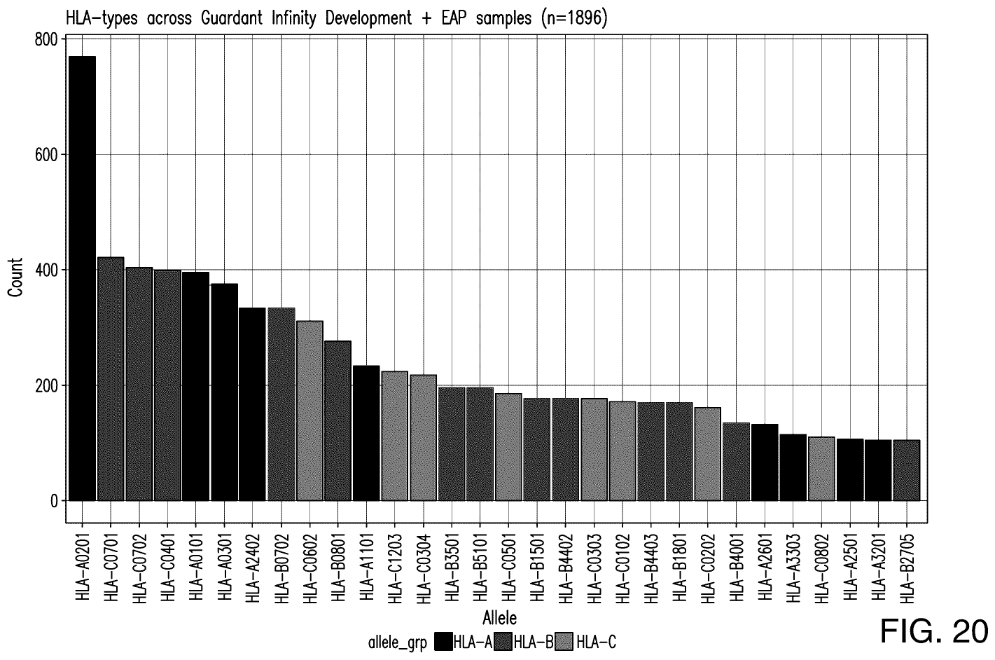

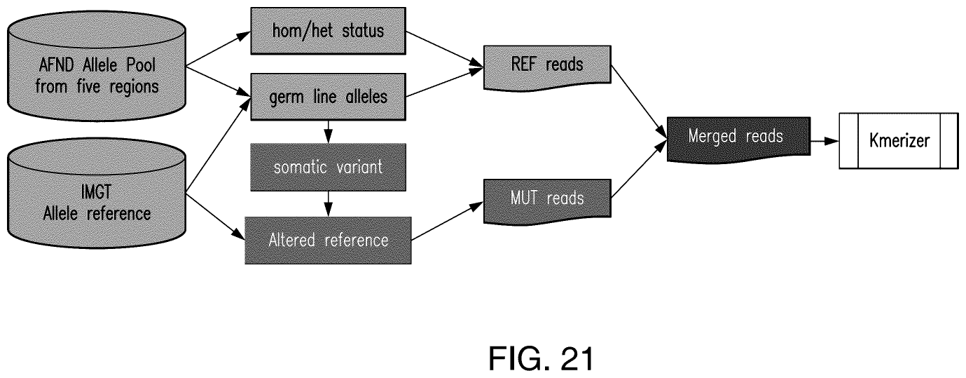

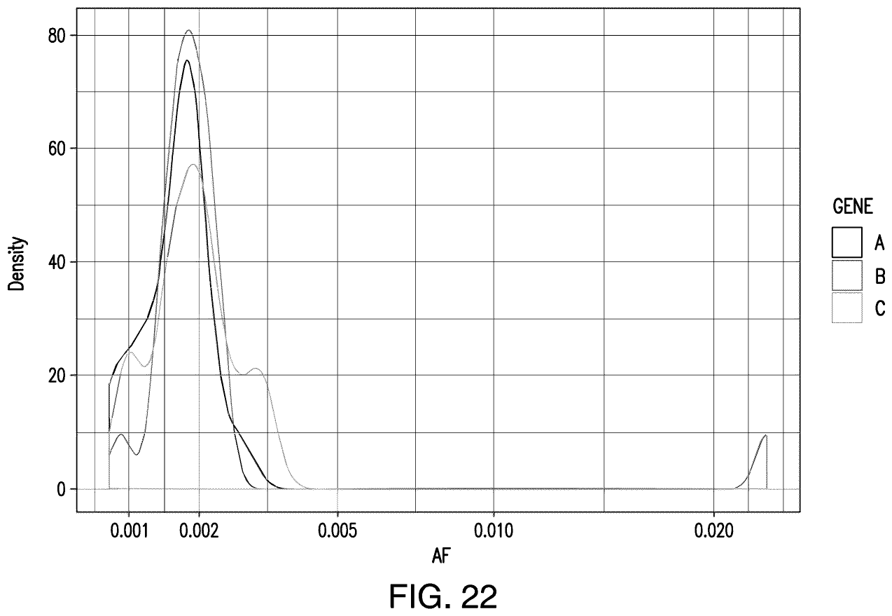

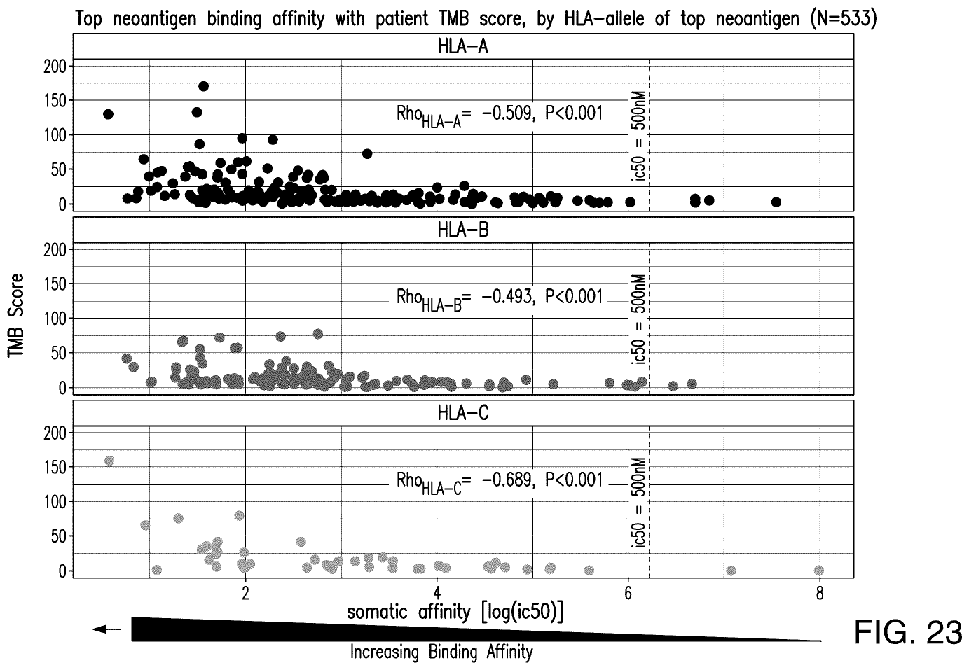

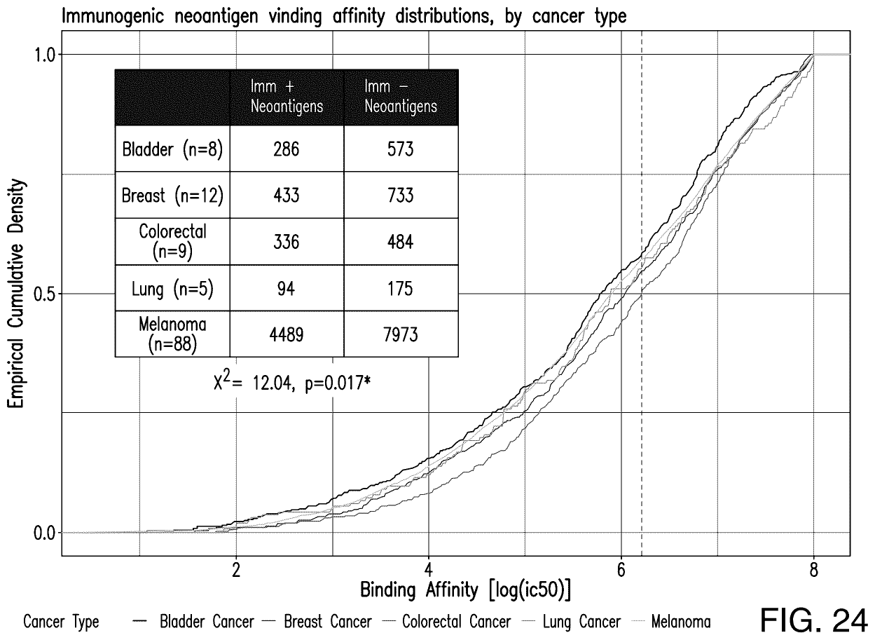

HLA and KIR sequences exhibit a high level of homology between allele sets (FIG. 11). Kmerizer calls HLA and KIR alleles in a sample based on a set of known alleles. The module kmerizer can be run in two modes: build index mode or call alleles mode (FIG. 12). In the build index mode, running without a sample's bam file in input will cause the algorithm to generate a kmers map index file. In the call alleles mode, running with a sample's bam file in input will cause the algorithm to call alleles, based on the kmers map index file generated with the “build index” mode. The build index generates a kmers map based on a given kmer size and known alleles fasta file. The call alleles is based on a sample bam file and a kmers map is generated as done in the build index.

Kmerizer encode alleles as a tree using existing the HLA nomenclature system, where each leaf node represents an allele sequence and each leaf node propagates its count onto its parent's node. FIG. 13 is an example of the hierarchical structure of allele names. An HLA allele name is encoded by a string such as A*01:02:03:04, comprising a gene name (A, for HLA-A), and up to four sets of digits. When reviewing two allele names: matching the first two sets of digits—the alleles code for the same protein, but they may have different genomic content at the exon level; matching the first three sets of digits—the alleles have the same genomic content at the exon level (their bases are identical in the exons); and matching all four sets of digits—the alleles have the same genomic content both in the exon and the introns. For KIR, when reviewing two allele names: matching the first three digits—the alleles code for the same protein; and matching the first five digits—the alleles have the same genomic content at the exon level (their bases are identical in the exons). This system can be configured to emit the most likely candidate alleles based on the desired solution: for example, classifying based on the second level yield allele types at peptide resolution; classifying based on the third/fourth level yield allele types at nucleic acid

Kmerizer has been validated in HLA typing (FIG. 15) and KIR typing (FIG. 16) with simulated samples. Simulation randomly picked 2 alleles in each locus from IMGT database and simulated the allele reads from NGS with sequencing errors. In a separate evaluation simulation challenged Kmerizer with allele pairs with sequence identity over 95%. We also executed HISAT-genotype on the same set of samples for method comparison.

The typing performance of kmerizer on gDNA sourced from cell lines validated with orthogonal methods was also performed. The HLA typing performance of kmerizer using 19 cell line samples sourced from Fred Hutch Anthropology Reference Panel (FIG. 17) were evaluated, where 15 samples sourced from sonicated gDNA and 4 samples sourced from cell line media. Finally the typing concordance of kmerizer was evaluated on plasma cfDNA collected from healthy donors as compared with buffy coat gDNA.

Of the 19 cell lines and 12 plasma samples for HLA typing (A, B, C, and DQB1 loci), kmerizer delivered 100% sensitivity with 98% specificity. On the simulated dataset, Kmerizer achieved 99% sensitivity and specificity on all the MHC class1 and class 2 loci, and 90% sensitivity and specificity on all KIR loci, for both homozygous and heterozygous pairs. The allele caller kmerizer also demonstrated a lighter footprint on computational resource need: one deep-sequencing plasma sample on average can be processed in less than 2 minutes which is about 15 times faster than the most commonly used HLA typing tool HISAT-genotype, which does not support KIR typing.

### Conclusions

Fragment size in cfDNA is shorter than in genomic DNA, and existing state of the art methods like HISAT-genotype have limited capability in calling alleles accurately. Kmerizer, a novel algorithm, was developed and it is capable of typing HLA and KIR alleles with cfDNA like fragments.

Kmerizer achieves high sensitivity and specificity on both simulated data and well-characterized cell lines, and it achieves high concordance between plasma and buffy coat DNA.

The simplicity of Kmerizer enables transparency between the sequencing data and the final reported alleles, and it has a fast turnaround time with minimal hardware requirements.

As utilization of ICIs increases, the use of genetic and genomic information to accurately identify patients more likely to respond to ICIs is critical. The ability to type HLA/KIR in cfDNA workflow is important in delivering cost effective and comprehensive cancer care with liquid biopsy.

### Example 2

HLA germline genotypes and somatic mutations show great promise as emerging biomarkers for immune checkpoint inhibitors (ICIs) and understanding patient prognosis. Multiple studies have shown that HLA homozygosity or loss-of-function somatic mutations negatively correlate with ICI response rate. High HLA fidelity can be used to accurately model patient neoantigen landscapes and provide further prognostic insight for these patients, and identify those who may benefit particularly from IO-treatment.

Additional data is presented on the methods and systems described herein, referred to as Kmerizer, configured to perform HLA germline typing and/or somatic mutation detection from cfDNA input material. These results can be used for in silico neoantigen and patient outcome prediction.

### Methods and Results

Kmerizer may leverage the high depth coverage of targeted sequencing to rapidly identify germline alleles by matching k-mers from the input reads to the k-mers of known HLA and KIR alleles. Realignment of reads onto the called germlines may be followed by somatic variant calling.

MHC-I germline allele calls may be combined with patient mutation data to generate in silico TCR binding affinity predictions using, for example, netMHC-4.01. These predictions may be compared across cohorts to assess how cancer type and bTMB vary with the predicted neoantigens and TCR binding affinity.

Of 19 cell lines, 12 plasma samples and eight gDNA samples with confirmed HLA typing information, Kmerizer delivered high sensitivity and specificity on both MHC-I and II genes based on between methylation epigenomic detection cfDNA sequencing data. For homozygous and heterozygous status, high accuracy on class I and II genes is achieved (Table 2). Table 2 shows Kmerizer germline allele calling performance on datasets with confirmed HLA typing information.

TABLE 2

| | | | MHC Class I | | | MHC Class II | | |
|---|---|---|---|---|---|---|---|---|
| Sample | Sample | N | allele | allele | genotype | allele | allele | genotype |
| Type | Size | Alleles | sensitivity | specificity | accuracy | sensitivity | specificity | accuracy |
| cell | 19 | 133 | 100% | 99.02% | 98.25% | 100% | 100% | 100% |
| line | | | | | | | | |
| plasma | 12 | 177 | 98.57% | 98.57% | 100% | 99.07% | 99.07% | 100% |
| gDNA | 8 | 135 | 100% | 100% | 100% | 100% | 97.75% | 96.08% |
| Total | 39 | 445 | 99.54% | 99.08% | 99.15% | 99.56% | 98.68% | 98.46% |

HLA germline allele prevalence among between methylation epigenomic detection samples is consistent with reference cohorts of similar geographic origin in MHC class I genes (FIG. 18). FIG. 18 shows HLA germline allele prevalence comparison between between methylation epigenomic detection samples (n=2004) and Allele Frequency Net Database (AFND) genotype data from different geographic regions (Europe, n=1000; North America, n=496; North East Asia, n=1510; South Central America, n=1463; South East Asia, n=5266). Allele of particular clinical interest are highlighted by “X” above.

The counts of germline HLA alleles for which immunogenic neoantigens were predicted (ic50 rank<1%; subset to 9 mer length neoantigens) were plotted across between methylation epigenomic detection development samples. The resulting distribution shown in FIG. 19 generally resembles the overall germline HLA allele distribution for all between methylation epigenomic detection samples shown in FIG. 20, indicating there is no substantial bias in neoantigen predictions toward a particular set of alleles.

Notably, the count of alleles with netMHC supported immunogenic neoantigen predictions was compared against those without predictions (inset table; for between methylation epigenomic detection development samples only) and found a significant association between HLA allele group (HLA-A/B/C) and immunogenic prediction, with more immunogenic predictions in HLA-A alleles across patients than HLA-B and HLA-C alleles.

The Kmerizer somatic caller achieves >99.99% specificity per base as computed on 48 normal samples on MHC class I genes. For sensitivity evaluation, simulated sequencing data was generated with randomly selected somatic variants with expected allele frequency (AF) at 0.15˜0.2% (FIG. 21). FIG. 21 shows that germline allele(s) and homozygous/heterozygous status were randomly assigned based on AFND to generate germline reads using references from the IMGT/HLA database; somatic mutation was then randomly generated at an exonic position and mutant reads were then generated from the altered reference.

The Kmerizer somatic caller achieved 100% specificity and 97.5% aggregated sensitivity on class I genes for both variant detection and mutant molecule recovery (Table 3). Table 3 shows Kmerizer somatic allele calling performance on simulated data. Detected variants have observed AF range [0.08%, 2.3%] and median of 0.18% (FIG. 22). FIG. 22 shows variant allele frequency (AF) distribution of detected variants.

TABLE 3

| | Mutant Detection | | | Mutant Molecule Detection | | |
|---|---|---|---|---|---|---|
| | total | | | total | | |
| Gene | variant | detected | sensitivity | molecules | detected | sensitivity |
| HLA-A | 23 | 23 | 100% | 69 | 68 | 98.55% |
| HLA-B | 21 | 20 | 95.24% | 63 | 61 | 96.83% |
| HLA-C | 36 | 35 | 97.22% | 108 | 105 | 97.22% |
| Total | 80 | 78 | 97.5% | 240 | 234 | 97.5% |

Kmerizer leverages germline HLA calling for neoantigen prediction. 53,107 neoantigens were generated with netMHC-4.0 from 533 clinical cancer samples using matched germline allele calls and mutation data. Patient top ranked neoantigen TCR binding affinity was strongly associated with bTMB score determined by the between methylation epigenomic detection assay (ρ=−0.524, p<0.0001). Grouping patients by HLA allele associated with the strongest binding affinity prediction indicates this association is consistent across genes (FIG. 23). FIG. 23 shows TMB v. predicted neoepitope affinity across genes. Spearman correlations listed for each group.

In the absence of expression data, samples with average somatic neoantigen binding affinity greater than germline affinity (N=122) were selected.

Comparing the binding affinity (ic50) for the top predicted neoantigen binding affinity by patient against bTMB indicates a strong inverse correlation between TMB score and presumed TCR binding (Rhospearman=−0.524, p<0.0001).

To understand if this relationship is being driven by neoantigen predictions for only one or some HLA alleles, the patients were subset by HLA-allele of the top neoantigen prediction and the correlations were recalculated, finding that the same association holds across HLA genes (FIG. 23: HLA-A/B/C; plot right)

Furthermore, it was determined that this relationship persists across alleles within HLA genes. Looking at HLA-A alleles with >5% of the total top HLA-A neoantigen predictions identified 4 alleles: HLA-A*02:01, HLA-A*03:01, HLA-A*11:01, and HLA-A*31:01. Plotting samples with the strongest potential binding affinity neoantigens in these alleles against patient bTMB indicates that the association with increasing bTMB with stronger binding affinity holds for all 4 alleles (RhoHLA-A*02:01=−0.683, p<0.0001; RhoHLA-A*03:01=−0.485, p<0.005; RhoHLA-A*11:01=−0.682, p<0.0005; RhoHLA-A*02:01=−0.453, p=0.103)

The rate of immunogenic neoantigens (ic50 rank<2%) varied significantly across sample cancer types (x2=12.04, p=0.017; FIG. 24, inset table). FIG. 24 shows the distribution of neoantigen binding affinities by cancer type. Comparison of the immunogenic neoantigen binding affinities across cancer types identified bladder and melanoma cancer samples as those with the highest immunogenic potential and colorectal cancers as immunologically cold, on average (ks-test, p<0.005; FIG. 24; stronger binding denoted by smaller ic50 score).

The dev samples were filtered to just those with median predicted neoantigen TCR affinities greater than those of the matched median germline TCR affinities. In the absence of expression data to identify mutated peptide expression levels, this filtering helps subset patients more likely to have a neoantigen that would stimulate CD8+ T-cell response compared to the background ‘noise’ of the germline produced antigens.

To determine whether there was a cancer-type specific effect on the rate of immunogenic neoantigen targets, all predictions were classified as either immunogenic (“strong binding”) or non-immunogenic (“likely no CD8+ binding”) based on netMHC percentile rank (rank<1%). Rates of immunogenic neoantigen targets by cancer type were compared via chi-squared test, finding a significant effect (FIG. 24: X2=12.04, p=0.017; inset table).

A cumulative density plot of the immunogenic neoantigens by cancer type (FIG. 24, right) demonstrates the immuno-stimulating potential of each group (affinity is represented by ic50, meaning lower values represent stronger TCR binding potential). These distributions suggest that, in this cohort, melanoma and bladder cancers have an average higher immunogenic potential while colorectal cancers have the lowest potential (likeliest to be “cold” tumors).

### Conclusion

The integration of Kmerizer into between methylation epigenomic detection enables accurate HLA germline and somatic detection and neoantigen prediction. Kmerizer demonstrated that HLA germline prevalence is correlated with public data. Kmerizer demonstrated HLA somatic specificity in 48 normal samples, sensitivity in simulation. Kmerizer demonstrated neoantigen prediction analysis in clinical samples.

Non-limiting embodiments are described below:

Embodiment 1: A method comprising determining a plurality of known allele sequences, determining a plurality of sequence reads of a target region of a chromosome of a subject, wherein the target region of the chromosome comprises one or more loci, aligning the plurality of sequence reads to the plurality of known allele sequences, determining, based on the alignment, for each known allele sequence of the plurality of known allele sequences, a number of sequence reads that aligned to each known allele sequence, and determining, based on the numbers of sequence reads that aligned to each known allele sequence, for the one or more loci, the known allele sequences present at the one or more loci.

Embodiment 2: The method of embodiment 1, wherein the plurality of known allele sequences comprise a plurality of known human leukocyte antigen (HLA) allele sequences or a plurality of known killer cell immunoglobulin-like receptor (KIR) region allele sequences.

Embodiment 3: The method of embodiment 1, further comprising obtaining a sample from the subject and sequencing the sample to obtain the plurality of sequence reads of the target region of the chromosome.

Embodiment 4: The method of embodiment 1, further comprising determining, based on the alignment, for each read of the plurality of sequence reads, one or more known allele sequences to which each read aligns.

Embodiment 5: The method of embodiment 1, wherein the target region comprises one or more of the following genes: HLA-A, HLA-B, HLA-C, HLA-E, HLA-F, HLA-G, HLA-H, HLA-J, HLA-K, HLA-L, HLA-N, HLA-P, HLA-S, HLA-T, HLA-U, HLA-V, HLA-W, HLA-DRA, HLA-DRB1, HLA-DRB2, HLA-DRB3, HLA-DRB4, HLA-DRB5, HLA-DRB6, HLA-DRB7, HLA-DRB8, HLA-DRB9, HLA-DQA1, HLA-DQB1, HLA-DQA2, HLA-DQB2, HLA-DQB3, HLA-DOA, HLA-DOB, HLA-DMA, HLA-DMB, HLA-DPA1, HLA-DPB1, HLA-DPA2, HLA-DPB2, HLA-DPA3, HFE, TAP1, TAP2, PSMB9, PSMB8, MICB, MICA, MICC, MICD, MICE, KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, or KIR3DL3.

Embodiment 6: The method of embodiment 1, wherein the target region comprises chromosomal position 6p21 or chromosomal position 19q13.

Embodiment 7: The method of embodiment 1, wherein the target region comprises at least a portion of a major histocompatibility complex (MHC) region and the chromosome is human chromosome 6.

Embodiment 8: The method of embodiment 1, wherein the target region comprises at least a portion of a killer cell immunoglobulin-like receptor (KIR) region and the chromosome is human chromosome 19.

Embodiment 9: The method of embodiment 1, wherein determining the plurality of known allele sequences comprises retrieving sequence data for known alleles, determining, based on the sequence data for known alleles, a plurality of k-mer sequences and assembling the plurality of k-mer sequences into a data structure comprising a plurality of entries, wherein each entry comprises a k-mer sequence, a number of alleles containing the k-mer sequence, an allele identifier for each allele containing the k-mer sequence, and a start and stop position of the k-mer sequence in each allele.

Embodiment 10: The method of embodiment 1, wherein determining, based on the alignment, for each known allele sequence of the plurality of known allele sequences, a number of sequence reads that aligned to each known allele sequence comprises determining the number of sequence reads that aligned to each known allele sequence with 100% identity.

Embodiment 11: The method of embodiment 1, wherein determining, based on the numbers of sequence reads that aligned to each known allele sequence, for the one or more loci, the known allele sequences present at the one or more loci comprises determining one or more known allele sequences having a highest number of sequence reads aligned.

Embodiment 12: The method of embodiment 1, wherein the reads that aligned to each known allele sequence are grouped into read families, and the method further comprises, determining a number of sequence read families that are aligned to each known allele sequence.

Embodiment 13: The method of embodiment 12, wherein determining, for the one or more loci, the known allele sequences present at the one or more loci based on the numbers of sequence read families that aligned to each known allele sequence.

Embodiment 14: The method of embodiment 1, further comprising determining a length of a portion of each known allele sequence aligned to two or more sequence reads of the plurality of sequence reads.

Embodiment 15: The method of embodiment 1, further comprising sorting, for a locus, the known allele sequences present at the locus by the number of sequence reads that aligned to each known allele sequence, determining, for the locus, a first known allele sequence with a highest number of sequence reads aligned, inserting the first known allele sequence with the highest number of sequence reads aligned into a superset, determining one or more known allele sequences that aligned to reads that are a subset of the reads aligned to the first known allele sequence, and inserting the one or more known allele sequences into the superset.

Embodiment 16: The method of embodiment 15, wherein the superset comprises a graph data structure.

Embodiment 17: The method of embodiment 16, wherein the graph data structure comprises a directed acyclic graph.

Embodiment 18: The method of embodiment 16, wherein the graph data structure represents a Hasse diagram.

Embodiment 19: The method of embodiment 15, further comprising determining that the locus is associated with a single superset and determining the first known allele sequence of the single superset as the allele present at the locus.

Embodiment 20: The method of embodiment 15, further comprising determining a plurality of supersets for the locus.

Embodiment 21: The method of embodiment 20, further comprising determining, based on the plurality of supersets for the locus, two supersets with a cumulative largest number of distinct reads and determining the first known allele sequence of each of the two supersets as the alleles present at the locus.

Embodiment 22: The method of embodiment 1, further comprising, assisting in a communication of the known allele sequences present at the one or more loci to a medical provider.

Embodiment 23: The method of embodiment 1, wherein the known allele sequences present at the locus comprise a human leukocyte antigen (HLA) type at the locus or a killer cell immunoglobulin-like receptor (KIR) type at the locus.

Embodiment 24: The method of embodiment 1, further comprising obtaining a sample from a cell to be transplanted into the subject and sequencing the sample to obtain the plurality of sequence reads of the target region of the chromosome.

Embodiment 25: The method of embodiment 24, further comprising determining if an HLA type of the subject matches the HLA type at the locus.

Embodiment 26: The method of embodiment 25, further comprising transplanting the cell into the subject if the HLA type of the subject matches the HLA type at the locus.

Embodiment 27: The method of embodiment 25, further comprising transplanting the cell into the subject if the HLA type of the subject matches the HLA type at the locus and administering an agent that reduces a likelihood of transplant rejection in the subject.

Embodiment 28: The method of embodiment 1, further comprising determining that the subject is predisposed to developing rheumatoid arthritis (RA) wherein the known allele sequences present at the one or more loci comprise HLA-DRB1*04:01, *04:04, and *04:08; HLA-DRB1*04:05; HLA-DRB1*01:01 and *01:02; HLA-DRB1*14:02; HLA-DRB1*10:01; and HLA-DRB1*01:01, *04:01, *04:04, and *04:05.

Embodiment 29: The method of embodiment 1, further comprising determining that the subject is predisposed to developing multiple sclerosis (MS) wherein the known allele sequences present at the one or more loci comprise HLA-DRB1*15:01, HLA-DQB1*06:02, HLA-DRB1*01:08, HLA-DRB1*03:01, and/or HLA-DRB1*13:03.

Embodiment 30: The method of embodiment 1, further comprising determining that the subject is predisposed to developing systemic lupus erythematosus (SLE) wherein the known allele sequences present at the one or more loci comprise HLA-DRB1, HLA-DR2 (DRB1*15:01), HLA-DR3 (DRB1*03:01), HLA-DRB1*08:01, and/or HLA-DQA1*01:02.

Embodiment 31: The method of embodiment 1, further comprising determining that the subject is predisposed to developing Type 1 diabetes mellitus (TID) wherein the known allele sequences present at the one or more loci comprise HLA-DQB1*03:02 and/or DQB1*02:01.

Embodiment 32: The method of embodiment 1, further comprising determining that the subject is predisposed to developing Sjögren's syndrome (SS) wherein the known allele sequences present at the one or more loci comprise DQA1*05:01, DQB1*02:01, and DRB1*03:01.

Embodiment 33: The method of embodiment 1, further comprising determining that the subject is predisposed to developing that the subject is predisposed to developing celiac disease (CD) wherein the known allele sequences present at the one or more loci comprise HLA-DQ2 (encoded by HLA-DQA1*05:01-DQB1*02:01) and/or HLA-DQ8 (encoded by DQA1*03:01-DQB1*03:02).

Embodiment 34: The method of embodiment 1, further comprising determining that the subject is predisposed to developing preeclampsia wherein the known allele sequences present at the one or more loci comprise KIR2DL1.

Embodiment 35: The method of embodiment 1, further comprising determining that the subject is less likely to progress from HIV infection to AIDS wherein the known allele sequences present at the one or more loci comprise KIR3DS1.

Embodiment 36: The method of embodiment 1, further comprising determining that the subject is less likely to progress from HIV infection to AIDS wherein the known allele sequences present at the one or more loci comprise a combination of KIR3DS1 and HLA-Bw4.

Embodiment 37: The method of embodiment 1, further comprising determining that the subject is predisposed to developing an autoimmune disease wherein the known allele sequences present at the one or more loci comprise KIR2DS1.

Embodiment 38: The method of embodiment 1, further comprising determining that the subject is predisposed to developing Crohn's disease wherein the known allele sequences present at the one or more loci comprise KIR2DL2/KIR2DL3 heterozygosity.

Embodiment 39: The method of embodiment 1, further comprising determining that the subject is predisposed to developing Crohn's disease wherein the known allele sequences present at the one or more loci comprise a combination of KIR2DL2/KIR2DL3 heterozygosity and HLA-C2 allele.

Embodiment 40: The method of embodiment 1, further comprising determining whether the subject is a suitable candidate for a donor in an unrelated hematopoietic cell transplantation wherein the known allele sequences present at the one or more loci comprise a group B KIR haplotype.

Embodiment 41: A method comprising determining a plurality of known human leukocyte antigen (HLA) allele sequences, determining a plurality of sequence reads of a target region of a chromosome of a subject, wherein the target region of the chromosome comprises one or more loci, aligning the plurality of sequence reads to the plurality of known HLA allele sequences, determining, based on the alignment, for each known HLA allele sequence of the plurality of known HLA allele sequences, a number of sequence reads that aligned to each known HLA allele sequence, generating, based on the numbers of sequence reads that aligned to each known HLA allele sequence, one or more supersets of known HLA allele sequences, and determining, based on a number of distinct reads in the one or more supersets of known HLA allele sequences, for the one or more loci, the known HLA allele sequences present at the one or more loci.

Embodiment 42: A method comprising determining a plurality of known killer cell immunoglobulin-like receptor (KIR) allele sequences, determining a plurality of sequence reads of a target region of a chromosome of a subject, wherein the target region of the chromosome comprises one or more loci, aligning the plurality of sequence reads to the plurality of known KIR allele sequences, determining, based on the alignment, for each known KIR allele sequence of the plurality of known KIR allele sequences, a number of sequence reads that aligned to each known KIR allele sequence, generating, based on the numbers of sequence reads that aligned to each known KIR allele sequence, one or more supersets of known KIR allele sequences, and determining, based on a number of distinct reads in the one or more supersets of known KIR allele sequences, for the one or more loci, the known KIR allele sequences present at the one or more loci.

Embodiment 43: A method comprising determining a plurality of known allele sequences, determining a plurality of sequence reads of a target region of a chromosome of a subject, wherein the target region of the chromosome comprises one or more loci, aligning the plurality of sequence reads to the plurality of known allele sequences, determining, based on the alignment, for each known allele sequence of the plurality of known allele sequences, a number of sequence reads that aligned to each known allele sequence, generating, based on the numbers of sequence reads that aligned to each known allele sequence, one or more supersets of known allele sequences, and determining, based on a number of distinct reads in the one or more supersets of known allele sequences, for the one or more loci, the known allele sequences present at the one or more loci.

Embodiment 44: One or more non-transitory computer-readable media storing processor-executable instructions thereon that, when executed by a processor, cause the processor to perform the methods of any of embodiments 1-43.

Embodiment 45: A system comprising a computing device configured to perform the methods of any of embodiments 1-43 and an output device configured to output an indication of the known allele sequences present at the one or more loci.

Embodiment 46: An apparatus, comprising one or more processors and memory storing processor-executable instructions that, when executed by the one or more processors, cause the apparatus to perform the methods of any of embodiments 1-43.

Embodiment 47: A method comprising determining a plurality of sequence reads or read pairs of a target region of a chromosome of a subject, wherein the target region of the chromosome comprises one or more loci, generating alignments of the plurality of sequence reads or read pairs to a plurality of known sequences, generating alignments of the plurality of sequence reads or read pairs to a plurality of decoy sequences, comparing the alignments of a sequence read or read pair to the known sequences against the alignments of the sequence read or read pair against the decoy sequences, wherein the sequence read or read pair is associated with a known sequence when an alignment score of the sequence read or read pair against a known sequence is greater than an alignment score of the sequence read or read pair against a decoy sequence, and identifying the sequence read or read pair as a supporting sequence read or read pair a sequence of interest.

Embodiment 48: The method of embodiment 47, further comprising obtaining a sample from the subject and sequencing the sample to obtain the plurality of sequence reads or read pairs of the target region of the chromosome.

Embodiment 49: The method of embodiment 47, wherein the target region comprises one or more of the following genes: HLA-A, HLA-B, HLA-C, HLA-E, HLA-F, HLA-G, HLA-H, HLA-J, HLA-K, HLA-L, HLA-N, HLA-P, HLA-S, HLA-T, HLA-U, HLA-V, HLA-W, HLA-DRA, HLA-DRB1, HLA-DRB2, HLA-DRB3, HLA-DRB4, HLA-DRB5, HLA-DRB6, HLA-DRB7, HLA-DRB8, HLA-DRB9, HLA-DQA1, HLA-DQB1, HLA-DQA2, HLA-DQB2, HLA-DQB3, HLA-DOA, HLA-DOB, HLA-DMA, HLA-DMB, HLA-DPA1, HLA-DPB1, HLA-DPA2, HLA-DPB2, HLA-DPA3, HFE, TAP1, TAP2, PSMB9, PSMB8, MICB, MICA, MICC, MICD, MICE, KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, or KIR3DL3.

Embodiment 50: The method of embodiment 47, wherein the target region comprises chromosomal position 6p21 or chromosomal position 19q13.

Embodiment 51: The method of embodiment 47, wherein the target region comprises at least a portion of a major histocompatibility complex (MHC) region and the chromosome is human chromosome 6.

Embodiment 52: The method of embodiment 47, wherein the target region comprises at least a portion of a killer cell immunoglobulin-like receptor (KIR) region and the chromosome is human chromosome 19.

Embodiment 53: The method of embodiment 47, wherein the plurality of known sequences comprise a plurality of known human leukocyte antigen (HLA) allele sequences or a plurality of known human killer cell immunoglobulin-like receptor (KIR) region allele sequences.

Embodiment 54: The method of embodiment 47, wherein the plurality of decoy sequences comprises a plurality of non-human sequences.

Embodiment 55: The method of embodiment 54, wherein the plurality of non-human sequences comprises one or more of a plurality of bovine sequences, a plurality of rat sequences, or a plurality of microbial sequences.

Embodiment 56: The method of embodiment 47, wherein generating alignments of the plurality of sequence reads or read pairs to the plurality of known sequences comprises determining, based on the alignments, for a sequence read or a read pair of the plurality of sequence reads or read pairs, one or more known sequences to which each sequence read or each read of the read pair aligns with no mismatch or indel.

Embodiment 57: The method of embodiment 56, further comprising determining, based on the alignments, for each known sequence of the plurality of known sequences, a number of sequence reads or a number of read pairs that aligned to each known sequence and determining, based on the number of sequence reads or the number of read pairs that aligned to each known sequence, for the one or more loci, the known sequences present at the one or more loci.

Embodiment 58: The method of embodiment 47, wherein generating alignments of the plurality of sequence reads or read pairs to a plurality of decoy sequences comprises determining, based on the alignments, for the sequence read or the read pair, one or more decoy sequences to which each sequence read or read pair aligns with no mismatch or indel and discarding the sequence read or read pair.

Embodiment 59: The method of embodiment 47, wherein generating alignments of the plurality of sequence reads or read pairs to a plurality of decoy sequences comprises determining, based on the alignments, for the sequence read or read pair, one or more non-human decoy sequences to which each sequence read or read pair aligns with no mismatch or indel and identifying the sequence read or read pair as originating from a contaminated sample.

Embodiment 60: The method of embodiment 47, wherein generating the alignments of the plurality of sequence reads or read pairs to a plurality of known sequences comprises determining, based on the alignments, for a sequence read or read pair, one or more known sequences to which each sequence read or read pair aligns with at least one mismatch or indel and generating the alignment score against the known sequence.

Embodiment 61: The method of embodiment 47, wherein generating the alignments of the plurality of sequence reads or read pairs to a plurality of decoy sequences comprises determining, based on the alignments, for the sequence read or read pair, one or more decoy sequences to which each sequence read or read pair aligns with at least one mismatch or indel and generating the alignment score against the decoy sequence.

Embodiment 62: The method of embodiment 47 wherein the alignment score against the known sequence and the alignment score against the decoy sequence each comprise at least one mismatch and/or indel.

Embodiment 63: The method of embodiment 47, wherein identifying the sequence read or read pair as a supporting sequence read or read pair of a sequence of interest comprises identifying, based on aligning the sequence read or read pair to one or more reference variants, a reference variant of the one or more reference variants as the candidate variant.

Embodiment 64: The method of embodiment 47, wherein identifying the sequence read or read pair as a supporting sequence read or read pair of a sequence of interest comprises identifying, based on aligning the sequence read or read pair to one or more non-human reference variants, a non-human reference variant of the one or more non-human reference variants as the candidate variant and identifying the sequence read or read pair as originating from a contaminated sample.

Embodiment 65: The method of embodiment 47, wherein generating alignments of the plurality sequence reads or read pairs to a plurality of known sequences comprises determining a read pair aligns to at least two sequences of the plurality of known sequences and selecting one known sequence of the at least two known sequences.

Embodiment 66: The method of embodiment 47, wherein generating alignments of the plurality sequence reads or read pairs to a plurality of decoy sequences comprises determining a read pair align to at least two decoy sequences of the plurality of decoy sequences and selecting one decoy sequence of the at least two decoy sequences.

Embodiment 67: A method comprising determining a plurality of sequence reads or read pairs of a target region of a chromosome of a subject, wherein the target region of the chromosome comprises one or more loci, generating alignments of the plurality of sequence reads or read pairs to a plurality of known allele sequences, generating alignments of the plurality of sequence reads or read pairs to a plurality of decoy allele sequences, comparing the alignments of a sequence read or read pair to the known alleles against the alignments of the sequence read or read pair against the decoy allele sequences, wherein the sequence read or read pair is associated with a germline allele when an alignment score of the sequence read or read pair against a known allele sequence is greater than an alignment score of the sequence read or read pair against a decoy allele sequence, and identifying the sequence read or read pair as a supporting sequence read or read pair for a candidate somatic variant.

Embodiment 68: The method of embodiment 67, further comprising obtaining a sample from the subject and sequencing the sample to obtain the plurality of sequence reads or read pairs of the target region of the chromosome.

Embodiment 69: The method of embodiment 67, wherein the target region comprises one or more of the following genes: HLA-A, HLA-B, HLA-C, HLA-E, HLA-F, HLA-G, HLA-H, HLA-J, HLA-K, HLA-L, HLA-N, HLA-P, HLA-S, HLA-T, HLA-U, HLA-V, HLA-W, HLA-DRA, HLA-DRB1, HLA-DRB2, HLA-DRB3, HLA-DRB4, HLA-DRB5, HLA-DRB6, HLA-DRB7, HLA-DRB8, HLA-DRB9, HLA-DQA1, HLA-DQB1, HLA-DQA2, HLA-DQB2, HLA-DQB3, HLA-DOA, HLA-DOB, HLA-DMA, HLA-DMB, HLA-DPA1, HLA-DPB1, HLA-DPA2, HLA-DPB2, HLA-DPA3, HFE, TAP1, TAP2, PSMB9, PSMB8, MICB, MICA, MICC, MICD, MICE, KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, or KIR3DL3.

Embodiment 70: The method of embodiment 67, wherein the target region comprises chromosomal position 6p21 or chromosomal position 19q13.

Embodiment 71: The method of embodiment 67, wherein the target region comprises at least a portion of a major histocompatibility complex (MHC) region and the chromosome is human chromosome 6.

Embodiment 72: The method of embodiment 67, wherein the target region comprises at least a portion of a killer cell immunoglobulin-like receptor (KIR) region and the chromosome is human chromosome 19.

Embodiment 73: The method of embodiment 67, wherein the plurality of known allele sequences comprises a plurality of known human leukocyte antigen (HLA) allele sequences or a plurality of known human killer cell immunoglobulin-like receptor (KIR) region allele sequences.

Embodiment 74: The method of embodiment 67, wherein the plurality of decoy allele sequences comprises a plurality of non-human sequences.

Embodiment 75: The method of embodiment 74, wherein the plurality of non-human sequences comprises one or more of a plurality of bovine sequences, a plurality of rat sequences, or a plurality of microbial sequences.

Embodiment 76: The method of embodiment 67, wherein generating alignments of the plurality of sequence reads or read pairs to the plurality of known allele sequences comprises determining, based on the alignments, for a sequence read or a read pair of the plurality of sequence reads or read pairs, one or more known allele sequences to which each sequence read or each read of the read pair aligns with no mismatch or indel.

Embodiment 77: The method of embodiment 76, further comprising determining, based on the alignments, for each known allele sequence of the plurality of known allele sequences, a number of sequence reads or a number of read pairs that aligned to each known allele sequence and determining, based on the number of sequence reads or the number of read pairs that aligned to each known allele sequence, for the one or more loci, the known allele sequences present at the one or more loci.

Embodiment 78: The method of embodiment 67, wherein generating alignments of the plurality of sequence reads or read pairs to a plurality of decoy allele sequences comprises determining, based on the alignments, for the sequence read or the read pair, one or more decoy allele sequences to which each sequence read or read pair aligns with no mismatch or indel and discarding the sequence read or read pair.

Embodiment 79: The method of embodiment 67, wherein generating alignments of the plurality of sequence reads or read pairs to a plurality of decoy allele sequences comprises determining, based on the alignments, for the sequence read or read pair, one or more non-human decoy allele sequences to which each sequence read or read pair aligns with no mismatch or indel and identifying the sequence read or read pair as originating from a contaminated sample.

Embodiment 80: The method of embodiment 67, wherein generating the alignments of the plurality of sequence reads or read pairs to a plurality of known allele sequences comprises determining, based on the alignments, for a sequence read or read pair, one or more known allele sequences to which each sequence read or read pair aligns with at least one mismatch or indel and generating the alignment score against the known allele sequence.

Embodiment 81: The method of embodiment 67, wherein generating the alignments of the plurality of sequence reads or read pairs to a plurality of decoy allele sequences comprises determining, based on the alignments, for the sequence read or read pair, one or more decoy allele sequences to which each sequence read or read pair aligns with at least one mismatch or indel and generating the alignment score against the decoy allele sequence.

Embodiment 82: The method of embodiment 67 wherein the alignment score against the known allele sequence and the alignment score against the decoy allele sequence each comprise at least one mismatch and/or indel.

Embodiment 83: The method of embodiment 67, wherein identifying the sequence read or read pair as a supporting sequence read or read pair of a sequence of interest comprises identifying, based on aligning the sequence read or read pair to one or more reference variants, a reference variant of the one or more reference variants as the candidate variant.

Embodiment 84: The method of embodiment 67, wherein identifying the sequence read or read pair as a supporting sequence read or read pair of a sequence of interest comprises identifying, based on aligning the sequence read or read pair to one or more non-human reference variants, a non-human reference variant of the one or more non-human reference variants as the candidate variant and identifying the sequence read or read pair as originating from a contaminated sample.

Embodiment 85: The method of embodiment 67, wherein generating alignments of the plurality sequence reads or read pairs to a plurality of known allele sequences comprises determining a read pair aligns to at least two sequences of the plurality of known allele sequences and selecting one known allele sequence of the at least two known allele sequences.

Embodiment 86: The method of embodiment 67, wherein generating alignments of the plurality sequence reads or read pairs to a plurality of decoy allele sequences comprises determining a read pair align to at least two decoy allele sequences of the plurality of decoy allele sequences and selecting one decoy allele sequence of the at least two decoy allele sequences.

Embodiment 89: A method comprising determining a plurality of pairs of sequence reads of a target region of a chromosome of a subject, wherein the target region of the chromosome comprises one or more loci, generating a germline alignment of the plurality of pairs of sequence reads to a plurality of known allele sequences, generating a decoy alignment of the plurality of pairs of sequence reads to a plurality of decoy allele sequences, determining a pair of sequence reads of the plurality of pairs of sequence reads with both a germline alignment with at least one mismatch and/or indel and a decoy alignment with at least one mismatch and/or indel, wherein the pair of sequence reads is associated with a germline alignment score greater than a decoy alignment score, and identifying the pair of sequence reads as a candidate somatic variant.

Embodiment 90: The method of embodiment 89, further comprising obtaining a sample from the subject and sequencing the sample to obtain the plurality of pairs of sequence reads of the target region of the chromosome.

Embodiment 91: The method of embodiment 89, wherein the target region comprises one or more of the following genes: HLA-A, HLA-B, HLA-C, HLA-E, HLA-F, HLA-G, HLA-H, HLA-J, HLA-K, HLA-L, HLA-N, HLA-P, HLA-S, HLA-T, HLA-U, HLA-V, HLA-W, HLA-DRA, HLA-DRB1, HLA-DRB2, HLA-DRB3, HLA-DRB4, HLA-DRB5, HLA-DRB6, HLA-DRB7, HLA-DRB8, HLA-DRB9, HLA-DQA1, HLA-DQB1, HLA-DQA2, HLA-DQB2, HLA-DQB3, HLA-DOA, HLA-DOB, HLA-DMA, HLA-DMB, HLA-DPA1, HLA-DPB1, HLA-DPA2, HLA-DPB2, HLA-DPA3, HFE, TAP1, TAP2, PSMB9, PSMB8, MICB, MICA, MICC, MICD, MICE, KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, or KIR3DL3.

Embodiment 92: The method of embodiment 89, wherein the target region comprises chromosomal position 6p21 or chromosomal position 19q13.

Embodiment 93: The method of embodiment 89, wherein the target region comprises at least a portion of a major histocompatibility complex (MHC) region and the chromosome is human chromosome 6.

Embodiment 94: The method of embodiment 89, wherein the target region comprises at least a portion of a killer cell immunoglobulin-like receptor (KIR) region and the chromosome is human chromosome 19.

Embodiment 95: The method of embodiment 89, wherein the plurality of known allele sequences comprise a plurality of known human leukocyte antigen (HLA) allele sequences or a plurality of known human killer cell immunoglobulin-like receptor (KIR) region allele sequences.

Embodiment 96: The method of embodiment 89, wherein the plurality of decoy sequences comprises a plurality of non-human sequences.

Embodiment 97: The method of embodiment 96, wherein the plurality of non-human sequences comprises one or more of a plurality of bovine sequences, a plurality of rat sequences, or a plurality of microbial sequences.

Embodiment 98: The method of embodiment 89, wherein generating the germline alignment of the plurality of pairs of sequence reads to a plurality of known allele sequences comprises determining, based on the germline alignment, for a pair of sequence reads of the plurality of pairs of sequence reads, one or more known allele sequences to which each read of the pair of sequence reads aligns with no mismatch or indel.

Embodiment 99: The method of embodiment 98, further comprising determining, based on the germline alignment, for each known allele sequence of the plurality of known allele sequences, a number of pairs of sequence reads of the plurality of pairs of sequence reads that aligned to each known allele sequence and determining, based on the numbers of pairs of sequence reads that aligned to each known allele sequence, for the one or more loci, the known allele sequences present at the one or more loci.

Embodiment 100: The method of embodiment 89, wherein generating the decoy alignment of the plurality of pairs of sequence reads to a plurality of decoy allele sequences comprises determining, based on the decoy alignment, for the pair of sequence reads of the plurality of pairs of sequence reads, one or more decoy allele sequences to which each read of the pair of sequence reads aligns with no mismatch or indel and discarding the pair of sequence reads.

Embodiment 101: The method of embodiment 89, wherein generating the decoy alignment of the plurality of pairs of sequence reads to a plurality of decoy allele sequences comprises determining, based on the decoy alignment, for the pair of sequence reads of the plurality of pairs of sequence reads, one or more non-human decoy sequences to which each read of the pair of sequence reads aligns with no mismatch or indel and identifying the plurality of pairs of sequence reads as originating from a contaminated sample.

Embodiment 102: The method of embodiment 89, wherein generating the germline alignment of the plurality of pairs of sequence reads to a plurality of known allele sequences comprises determining, based on the germline alignment, for a pair of sequence reads of the plurality of pairs of sequence reads, one or more known allele sequences to which each read of the pair of sequence reads aligns with at least one mismatch or indel and generating the germline alignment score.

Embodiment 103: The method of embodiment 102, wherein generating the decoy alignment of the plurality of pairs of sequence reads to a plurality of decoy allele sequences comprises determining, based on the decoy alignment, for the pair of sequence reads of the plurality of pairs of sequence reads, one or more decoy allele sequences to which each read of the pair of sequence reads aligns with at least one mismatch or indel and generating the decoy alignment score

Embodiment 104: The method of embodiment 89, wherein identifying the pair of sequence reads as a candidate somatic variant comprises identifying, based on aligning the pair of sequence reads to one or more reference variants, a reference variant of the one or more reference variants as the candidate variant.

Embodiment 105: The method of embodiment 89, wherein identifying the pair of sequence reads as a candidate somatic variant comprises identifying, based on aligning the pair of sequence reads to one or more non-human reference variants, a non-human reference variant of the one or more non-human reference variants as the candidate variant and identifying the plurality of pairs of sequence reads as originating from a contaminated sample.

Embodiment 106: The method of embodiment 89, wherein generating a germline alignment of the plurality of pairs of sequence reads to a plurality of known allele sequences comprises determining a pair of sequence reads aligns to at least two allele sequences of the plurality of known allele sequences and selecting one known allele sequence of the at least two allele sequences.

Embodiment 107: The method of embodiment 89, wherein generating a decoy alignment of the plurality of pairs of sequence reads to a plurality of decoy allele sequences comprises determining a pair of sequence reads align to at least two decoy allele sequences of the plurality of decoy allele sequences and selecting one decoy allele sequence of the at least two decoy allele sequences.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Features which are described in the context of separate aspects and embodiments of the invention may be used together and/or be interchangeable. Similarly, features described in the context of a single embodiment may also be provided separately or in any suitable sub-combination. Any feature, step, element, embodiment, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

What is claimed is:

1. A method comprising:
determining a plurality of pairs of sequence reads of a target region of a chromosome of a subject, wherein the target region of the chromosome comprises one or more loci;
generating a germline alignment of the plurality of pairs of sequence reads to a plurality of known allele sequences;
generating a decoy alignment of the plurality of pairs of sequence reads to a plurality of decoy allele sequences;
determining a pair of sequence reads of the plurality of pairs of sequence reads with both a germline alignment with at least one mismatch and/or indel and a decoy alignment with at least one mismatch and/or indel, wherein the pair of sequence reads is associated with a germline alignment score greater than a decoy alignment score; and
identifying the pair of sequence reads as a candidate somatic variant.

2. The method of claim 1, further comprising:
obtaining a sample from the subject; and
sequencing the sample to obtain the plurality of pairs of sequence reads of the target region of the chromosome.

3. The method of claim 1, wherein the target region comprises one or more of the following genes: HLA-A, HLA-B, HLA-C, HLA-E, HLA-F, HLA-G, HLA-H, HLA-J, HLA-K, HLA-L, HLA-N, HLA-P, HLA-S, HLA-T, HLA-U, HLA-V, HLA-W, HLA-DRA, HLA-DRB1, HLA-DRB2, HLA-DRB3, HLA-DRB4, HLA-DRB5, HLA-DRB6, HLA-DRB7, HLA-DRB8, HLA-DRB9, HLA-DQA1, HLA-DQB1, HLA-DQA2, HLA-DQB2, HLA-DQB3, HLA-DOA, HLA-DOB, HLA-DMA, HLA-DMB, HLA-DPA1, HLA-DPB1, HLA-DPA2, HLA-DPB2, HLA-DPA3, HFE, TAP1, TAP2, PSMB9, PSMB8, MICB, MICA, MICC, MICD, MICE, KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, or KIR3DL3.

4. The method of claim 1, wherein the target region comprises chromosomal position 6p21 or chromosomal position 19q13.

5. The method of claim 1, wherein the target region comprises at least a portion of a major histocompatibility complex (MHC) region and the chromosome is human chromosome 6.

6. The method of claim 1, wherein the target region comprises at least a portion of a killer cell immunoglobulin-like receptor (KIR) region and the chromosome is human chromosome 19.

7. The method of claim 1, wherein the plurality of known allele sequences comprise a plurality of known human leukocyte antigen (HLA) allele sequences or a plurality of known human killer cell immunoglobulin-like receptor (KIR) region allele sequences.

8. The method of claim 1, wherein the plurality of decoy sequences comprises a plurality of non-human sequences.

9. The method of claim 1, wherein generating the germline alignment of the plurality of pairs of sequence reads to a plurality of known allele sequences comprises:
determining, based on the germline alignment, for a pair of sequence reads of the plurality of pairs of sequence reads, one or more known allele sequences to which each read of the pair of sequence reads aligns with no mismatch or indel.

10. The method of claim 9, further comprising:
determining, based on the germline alignment, for each known allele sequence of the plurality of known allele sequences, a number of pairs of sequence reads of the plurality of pairs of sequence reads that aligned to each known allele sequence; and
determining, based on the numbers of pairs of sequence reads that aligned to each known allele sequence, for the one or more loci, the known allele sequences present at the one or more loci.

11. The method of claim 1, wherein generating the decoy alignment of the plurality of pairs of sequence reads to a plurality of decoy allele sequences comprises:
determining, based on the decoy alignment, for the pair of sequence reads of the plurality of pairs of sequence reads, one or more decoy allele sequences to which each read of the pair of sequence reads aligns with no mismatch or indel; and
discarding the pair of sequence reads.

12. The method of claim 1, wherein generating the decoy alignment of the plurality of pairs of sequence reads to a plurality of decoy allele sequences comprises:
determining, based on the decoy alignment, for the pair of sequence reads of the plurality of pairs of sequence reads, one or more non-human decoy sequences to which each read of the pair of sequence reads aligns with no mismatch or indel; and
identifying the plurality of pairs of sequence reads as originating from a contaminated sample.

13. The method of claim 1, wherein generating the germline alignment of the plurality of pairs of sequence reads to a plurality of known allele sequences comprises:
determining, based on the germline alignment, for a pair of sequence reads of the plurality of pairs of sequence reads, one or more known allele sequences to which each read of the pair of sequence reads aligns with at least one mismatch or indel; and
generating the germline alignment score.

14. The method of claim 13, wherein generating the decoy alignment of the plurality of pairs of sequence reads to a plurality of decoy allele sequences comprises:
determining, based on the decoy alignment, for the pair of sequence reads of the plurality of pairs of sequence reads, one or more decoy allele sequences to which each read of the pair of sequence reads aligns with at least one mismatch or indel; and
generating the decoy alignment score.

15. The method of claim 1, wherein identifying the pair of sequence reads as a candidate somatic variant comprises:
identifying, based on aligning the pair of sequence reads to one or more reference variants, a reference variant of the one or more reference variants as the candidate variant.

16. The method of claim 1, wherein identifying the pair of sequence reads as a candidate somatic variant comprises:
identifying, based on aligning the pair of sequence reads to one or more non-human reference variants, a non-human reference variant of the one or more non-human reference variants as the candidate variant; and
identifying the plurality of pairs of sequence reads as originating from a contaminated sample.

17. The method of claim 1, wherein generating a germline alignment of the plurality of pairs of sequence reads to a plurality of known allele sequences comprises:
determining a pair of sequence reads aligns to at least two allele sequences of the plurality of known allele sequences; and
selecting one known allele sequence of the at least two allele sequences.

18. The method of claim 1, wherein generating a decoy alignment of the plurality of pairs of sequence reads to a plurality of decoy allele sequences comprises:
determining a pair of sequence reads align to at least two decoy allele sequences of the plurality of decoy allele sequences; and
selecting one decoy allele sequence of the at least two decoy allele sequences.

19. A method comprising:
determining a plurality of sequence reads or read pairs of a target region of a chromosome of a subject, wherein the target region of the chromosome comprises one or more loci;
generating alignments of the plurality of sequence reads or read pairs to a plurality of known sequences;
generating alignments of the plurality of sequence reads or read pairs to a plurality of decoy sequences;
comparing the alignments of a sequence read or read pair to the known sequences against the alignments of the sequence read or read pair against the decoy sequences, wherein the sequence read or read pair is associated with a known sequence when an alignment score of the sequence read or read pair against a known sequence is greater than an alignment score of the sequence read or read pair against a decoy sequence; and
identifying the sequence read or read pair as a supporting sequence read or read pair of a sequence of interest.

20. The method of claim 19, further comprising:
obtaining a sample from the subject; and
sequencing the sample to obtain the plurality of sequence reads or read pairs of the target region of the chromosome.
